(19) United States
(12) Patent Application Publication
(10) Pub. No.: US 2021/0205363 A1
(43) Pub. Date: Jul. 8, 2021

(54) METHODS OF TREATMENT USING A GENETICALLY MODIFIED AUTOLOGOUS T CELL IMMUNOTHERAPY

(71) Applicant: PACT PHARMA, INC., South San Francisco, CA (US)

(72) Inventors: Barbara Sennino, San Francisco, CA (US); Bhamini Purandare, San Jose, CA (US); Stefanie Mandl-Cashman, San Francisco, CA (US); Gregg D. Fine, San Francisco, CA (US); Arati V. Rao, Palo Alto, CA (US); Todd Stallings-Schmitt, San Francisco, CA (US); Mark Walter Frohlich, Mercer Island, WA (US); Alex Franzusoff, El Granada, CA (US)

(73) Assignee: PACT PHARMA, INC., South San Francisco, CA (US)

(21) Appl. No.: 17/099,192

(22) Filed: Nov. 16, 2020

Related U.S. Application Data
(63) Continuation of application No. PCT/US2020/054732, filed on Oct. 8, 2020.
(60) Provisional application No. 62/912,545, filed on Oct. 8, 2019.

(51) Int. Cl. A61K 35/17; A61K 38/20; A61K 39/395; A61P 35/02

(52) U.S. Cl. CPC A61K 35/17; A61K 38/2013; A61P 35/02; A61K 39/3955; A61K 38/2086

(57) ABSTRACT

Methods of treating cancer with a precision genome engineered NeoTCR Product are described herein.

### CROSS-REFERENCE TO RELATED APPLICATIONS

This application is a Continuation of International Patent Application No.: PCT/US20/54732 filed Oct. 8, 2020, claims priority to U.S. Provisional Patent Application Ser. No. 62/912,545, filed Oct. 8, 2019, the content of each of which is incorporated by reference in its entirety, and to each of which priority is claimed.

### SEQUENCE LISTING

The present specification makes reference to a Sequence Listing (submitted electronically as a .txt file named “0875200166SL.txt” on Nov. 16, 2020). The .txt file was generated on Nov. 16, 2020 and is 1,075 bytes in size. The entire contents of the Sequence Listing are hereby incorporated by reference.

### BACKGROUND OF THE INVENTION

The clinical relevance of T cell immunity in the control of a diverse set of human cancers has been established. Clinical activity observed in immuno-oncology trials and with approved drugs often depends on unleashing a pre-existing intrinsic T cell immune response in each cancer patient. The unleashing of endogenous T cell immune responses following treatment with anti-PD1/anti-PD-L1 antibody drugs is routinely observed as rapid and durable tumor reduction following initiation of therapy. Treatment of patients with these immune checkpoint blockade therapies has resulted in improvements in overall survival in patients with advanced malignancies in a spectrum of tumor types (Robert, et al., 2015) (Herbst, et al., 2014) (Wolchok, et al., 2013) (Hamid, et al., 2013) (Brahmer, et al., 2012) (Topalian, et al., 2012). However, despite these successes of immunotherapies, a significant proportion of treated patients with solid tumors do not respond, signifying a high unmet need for patients to achieve clinical benefit from novel approaches to immunotherapy.

### SUMMARY OF THE INVENTION

The present disclosure provides a composition comprising: a) a first NeoTCR cell population comprising a first NeoTCR that binds a first neoantigen; b) a first NeoTCR cell population comprising a first NeoTCR that binds a first neoantigen, and a second NeoTCR cell population comprising a second NeoTCR that binds a second neoantigen; or c) a first NeoTCR cell population comprising a first NeoTCR that binds a first neoantigen, a second NeoTCR cell population comprising a second NeoTCR that binds a second neoantigen, and a third NeoTCR cell population comprising a third NeoTCR that binds a third neoantigen; wherein each NeoTCR is different from the others, and wherein each NeoTCR is derived from a patient.

In certain embodiments, the first, second, and/or third neoantigens are expressed by a single gene. In certain embodiments, the first, second, and/or third neoantigens are expressed by different genes. In certain embodiments, two of the first, second, and/or third neoantigens are expressed by a single gene. In certain embodiments, the first, second, and/or third NeoTCRs bind to a single major histocompatibility complex. In certain embodiments, the first, second, and/or third NeoTCRs bind to different major histocompatibility complexes. In certain embodiments, two of the first, second, and/or third NeoTCRs bind to a single major histocompatibility complex.

In certain embodiments, the composition comprises a pharmaceutically acceptable carrier. In certain embodiments, the composition comprises a cryopreservation agent. In certain embodiments, the composition comprises serum albumin. In certain embodiments, the composition comprises a crystalloid solution. In certain embodiments, the composition comprises Plasma-Lyte A, human serum album (HAS), and CryoStor® CS10.

The present disclosure provides a method of treating a cancer in a subject in need thereof, comprising administering a composition disclosed herein.

In certain embodiments, the composition comprises an amount of NeoTCR Cells of about 4×10⁸ cells, 1.33×10⁹ cells, or about 4×10⁹ cells. In certain embodiments, the composition comprises an amount of NeoTCR cells greater than about 4×10⁸ cells and less than about 1.33×10⁹ cells, greater than about 1.33×10⁹ cells and less than about 4×10⁹ cells, or greater than about 4×10⁹ cells. In certain embodiments, the composition comprises an amount of NeoTCR cells according to Table 4. In certain embodiments, the composition comprises an amount of NeoTCR cells according to Table 5. In certain embodiments, the composition is administered in a single dose. In certain embodiments, the composition is administered in multiple doses.

In certain embodiments, the method further comprises administering a combination agent. In certain embodiments, the combination agent is a cytokine, a PD-axis binding agent, a PD-1 binding agent, a PD-L1 binding agent, a PD-L2 binding agent, or a combination thereof. In certain embodiments, the cytokine is an IL-2 agent, an IL-7 agent, an IL-10 agent, an IL-12 agent, an IL-15 agent, an IL-18 agent, an IL-21 agent, or a combination thereof. In certain embodiments, the cytokine is an IL-2 agent. In certain embodiments, the cytokine is an IL-15 agent. In certain embodiments, the PD-axis binding agent comprises nivolumab, pembrolizumab, or atezolizumab.

In certain embodiments, the cancer is a liquid cancer or a solid cancer. In certain embodiments, the composition is administered following a pretreatment regime of fludarabine and cyclophosphamide.

The present disclosure provides a method of manufacturing a composition disclosed herein. The present disclosure also provides a kit for the administration of a composition disclosed herein.

The present disclosure provides a plurality of cells comprising a first modified cell comprising a first exogenous polynucleotide encoding a first NeoTCR binding a first antigen, wherein the first exogenous polynucleotide is integrated in an endogenous TRAC and/or TRBC locus of the first modified cell; a second modified cell comprising a second exogenous polynucleotide encoding a second NeoTCR binding a second antigen, wherein the second exogenous polynucleotide is integrated in an endogenous TRAC and/or TRBC locus of the second modified cell; a third modified cell comprising a third exogenous polynucleotide encoding a third NeoTCR binding a third antigen, wherein the third exogenous polynucleotide is integrated in an endogenous TRAC and/or TRBC locus of the third modified cell; or a combination thereof; and wherein the first, second and third NeoTCRs are patient derived and wherein the first, second and third modified cells are patient-derived primary cells.

In certain embodiments, the first, second, and third antigens are cancer antigens. In certain embodiments, the cancer antigens are neoantigens. In certain embodiments, the primary cells are lymphocytes. In certain embodiments, the primary cells are T cells, optionally wherein the T cells are: a) CD45RA+, CD62L+, CD28+, CD95−, CCR7+, and CD27+; b) CD45RA+, CD62L+, CD28+, CD95+, CD27+, CCR7+; or c) CD45RO+, CD62L+, CD28+, CD95+, CCR7+, CD27+, CD127+.

The present disclosure provides a composition comprising the plurality of cells disclosed herein. In certain embodiments, the composition further comprises a pharmaceutically acceptable excipient. In certain embodiments, the composition is administered to a patient in need thereof for the treatment of cancer.

In certain embodiments, the composition comprises a cryopreservation agent. In certain embodiments, the composition comprises serum albumin. In certain embodiments, the composition comprises a crystalloid solution. In certain embodiments, the composition comprises Plasma-Lyte A, human serum album (HAS), and CryoStor® CS10.

The present disclosure provides a method of treating a cancer in a subject in need thereof, the method comprising administering a plurality of cells, comprising: i) a first modified cell comprising a first exogenous polynucleotide encoding a first NeoTCR binding a first antigen, wherein the first exogenous polynucleotide is integrated in an endogenous TRAC and/or TRBC locus of the first modified cell; ii) a second modified cell comprising a second exogenous polynucleotide encoding a second NeoTCR binding a second antigen, wherein the second exogenous polynucleotide is integrated in an endogenous TRAC and/or TRBC locus of the second modified cell; iii) a third modified cell comprising a third exogenous polynucleotide encoding a third NeoTCR binding a third antigen, wherein the third exogenous polynucleotide is integrated in an endogenous TRAC and/or TRBC locus of the third modified cell; or iv) a combination thereof wherein the first, second and third NeoTCRs are patient derived and wherein the first, second and third modified cells are patient derived primary cells, thereby treating the cancer in the subject.

In certain embodiments, the first, second, and third antigens are cancer antigens. In certain embodiments, wherein the cancer antigens are neoantigens. In certain embodiments, the primary cells are lymphocytes. In certain embodiments, the primary cells are T cells, optionally wherein the T cells are: a) CD45RA+, CD62L+, CD28+, CD95−, CCR7+, and CD27+; b) CD45RA+, CD62L+, CD28+, CD95+, CD27+, CCR7+; or c) CD45RO+, CD62L+, CD28+, CD95+, CCR7+, CD27+, CD127+.

In certain embodiments, the method further comprises administering a combination agent. In certain embodiments, the combination agent is a cytokine, a PD-axis binding agent, a PD-1 binding agent, a PD-L1 binding agent, a PD-L2 binding agent, or a combination thereof. In certain embodiments, the cytokine is an IL-2 agent, an IL-7 agent, an IL-10 agent, an IL-12 agent, an IL-15 agent, an IL-18 agent, an IL-21 agent, or a combination thereof. In certain embodiments, the cytokine is an IL-2 agent. In certain embodiments, the cytokine is an IL-15 agent. In certain embodiments, the PD-axis binding agent comprises nivolumab, pembrolizumab, or atezolizumab. In certain embodiments, the cancer is a liquid cancer or a solid cancer.

The present disclosure provides a method of treating a cancer in a subject in need thereof, the method comprising: a) administering an effective amount of a modified cell comprising an exogenous polynucleotide encoding a NeoTCR binding a tumor antigen, wherein the exogenous polynucleotide is integrated in an endogenous TRAC and/or TRBC locus of the modified cell; and b) administering an effective amount of a combination agent; thereby treating the cancer in the subject.

In certain embodiments, the combination agent comprises a chemotherapeutic agent, an anti-hormonal agent, an endocrine therapeutic, a cytotoxic agent, a cytokine, a PD-axis binding agent, a PD-1 binding agent, a PD-L1 binding agent, a PD-L2 binding agent, or a combination thereof. In certain embodiments, the cytokine is an IL-2 agent, an IL-7 agent, an IL-10 agent, an IL-12 agent, an IL-15 agent, an IL-18 agent, an IL-21 agent, or a combination thereof. In certain embodiments, the cytokine is an IL-2 agent. In certain embodiments, the cytokine is an IL-15 agent. In certain embodiments, the PD-axis binding agent comprises nivolumab, pembrolizumab, or atezolizumab. In certain embodiments, the cancer is a liquid cancer or a solid cancer.

### BRIEF DESCRIPTION OF THE DRAWINGS

FIG. 1 shows representative images obtained with time-lapse live microscopy (Days 0, 1, and 2) show antigen-specific cytotoxic activity and proliferation by neo12-TCR T cells (a representative NeoTCR Product) co-cultured with target T cells expressing cognate neo12 peptide-HLA (right column), but not when co-cultured with target T cells expressing an irrelevant peptide (left column). Neo12 is a NeoTCR that was identified through tumor and blood screening and later precision genome engineered into T cells and made into a NeoTCR Product. Tumor cells used in these experiments expressed a variant of green fluorescent protein (GFP or ZsGreen) in a stable and homogeneous manner. These cells exhibit high uniform green fluorescence that allows identification of the cells by fluorescent microscopy. To detect real time apoptosis, a highly-selective phosphatidylserine cyanine fluorescent dye (IncuCyte Annexin V in red) was added to the co-culture. Tumor cells not displaying the appropriate antigen continue to grow in the presence of neo12-TCR T cells (left column). In contrast, the majority of neo12 antigen displaying tumor cells are apoptotic or dead within 2 days (right column). T cells are not labeled in this experiment, but antigen-specific proliferation can be appreciated visually by increased numbers of T cells over the course of 2 days (right column). Raw microscopy and microarray scans of the cell samples (loaded onto the single-cell barcode chip) and protein secretion data were analyzed using Isoplexis image processing software to determine the locations of chambers containing single cells and subsequently extract their secretion readouts. Data from empty cell chambers were used to measure the background intensity levels for each analyzed protein. Single-cell readouts were then normalized using the background readouts to determine significant secretions and compare profiles across assays. Isoplexis proprietary software and the R statistical package were used for statistical data analysis. T cells used in this study were generated from a healthy donor. Edited T cells were incubated with target tumor cells at a ratio of 2:1 prior to loading onto the IsoPlexis platform for effector protein secretion profile analysis.

FIG. 2 shows graphs showing the percentage of CD4 and CD8 T cells engineered to either express neo12 or F5 TCR, that secrete 2, 3, 4 or greater than-equal to 5 cytokines (shades of grey) when encountering cognate antigen. Cells were co-cultured with target T cells pulsed with no peptide, 10 nM or 100 nM specific peptide or with target T cells constitutively expressing pHLA on their surface (N: neo12 peptide-HLA-A2 receptor expressing tumor cells; M: MART1 peptide-HLA-A2 receptor expressing tumor cells). Secreted cytokine levels were assessed after 24 h of co-culture. The proportion of individual T cells from the entire T cell population that express two or more effector molecules upon stimulation are shown as different color shading in each bar. Cells secreting two or more effector proteins are considered polyfunctional.

FIG. 3 shows a representative longitudinal analysis of NeoE T cell response in a patient with melanoma treated with cancer vaccine.

FIGS. 4A and 4B show longitudinal monitoring of different TCRs obtained from PBMC in response to treatment with anti-PD-1 antibody. FIG. 4A shows the number of neoantigen-specific (neoE-specific) T cells per 10×10⁶ CD8⁺ T cells. FIG. 4B shows the mRNA expression of the neoantigens.

FIG. 5 shows a representative model of the functional characterization of the NeoTCR cells candidates on autologous tumor cells. In experiments performed using this functional characterization approach, neoTCR T cells were cocultured with autologous tumor cell line at product to target ratio (P:T) of 10:1. The NeoTCR cells were tested as single NeoTCR Product, a 2 NeoTCR Product composed of ½ of each of the two NeoTCR cells (i.e., an equal amount of each NeoTCR cell), or as a 3 NeoTCR Product composed ⅓ of each one of the three TCRs (i.e., an equal amount of each NeoTCR cell). The NeoTCR used in the NeoTCR Products were tested in combination targeted different neoepitopes and/or HLAs. The autologous tumor cells were transduced to express a red fluorescent protein and the amount of red confluency was then measured over time in the IncuCyte system. The decrease in the percentage of red fluorescent protein positive cells was used as a measure of the cytotoxic activity of the NeoTCR Products. Negative controls for this experiment were: (1) tumor cells with medium alone; (2) NeoTCR cells cocultured with tumor cell line from a different patient; (3) coculture of an unrelated NeoTCR (neo12) Product with the autologous tumor cell line.

FIGS. 6A and 6B show that the killing of autologous tumor cells with single NeoTCR Product is not efficient at the tested P:T Ratio of 30:1. FIG. 6A shows that none of the 1 NeoTCR Products show sustained killing over 100 hrs. In contrast, FIG. 6B shows that all of the 3 NeoTCR Products exhibit sustained killing over 100 hrs. RPMI M490 cells used as a control in both FIGS. 6A and 6B. While not all data is shown, in all 23 different NeoTCRs were used to make 1 NeoTCR Products, 2 NeoTCR Products, and 3 NeoTCR Products. The boxed 3 NeoTCR Products shown in FIG. 6B are the 3 NeoTCR Products used as the examples in FIG. 7 and FIG. 8.

FIGS. 7A-7D shows the 3 NeoTCR Product comprising NeoTCR 408, NeoTCR 409, and NeoTCR 429 as shown in FIG. 6B. FIG. 7A shows the single NeoTCR Products of each of the NeoTCRs compared to controls (the RPMI M490 and Neo12) and compared to the 3 NeoTCR Product comprising all three of the NeoTCRs (equal amounts of each of the NeoTCR 408 cells, NeoTCR 409 cells, and NeoTCR 429 cells). FIGS. 7B-7D show three graphs which show comparisons of the 1 NeoTCR Products compared to controls (the RPMI M490 and Neo12) and compared to the 2 NeoTCR Product comprising two of the NeoTCRs (equal amounts of each of the two NeoTCR cells). As shown in FIGS. 7A and 7B-7D, while the 2 NeoTCR Products exhibited killing, none of the 2 NeoTCR combinations exhibited the same degree of killing as the 3 NeoTCR Product. FIG. 7C shows that each of the NeoTCRs have different neoepitopes, that there is a diversity of HLAs, and difference in physiological properties of the NeoTCRs.

FIGS. 8A-8D show the 3 NeoTCR Product comprising NeoTCR 409, NeoTCR 429, and NeoTCR 421 as shown in FIG. 6B. FIG. 8A shows the single NeoTCR Products of each of the NeoTCRs compared to controls (the RPMI M490 and Neo12) and compared to the 3 NeoTCR Product comprising all three of the NeoTCRs (equal amounts of each of the NeoTCR 409 cells, NeoTCR 429 cells, and NeoTCR 421 cells). FIGS. 8B-8D show comparisons of the 1 NeoTCR Products compared to controls (the RPMI M490 and Neo12) and compared to the 2 NeoTCR Product comprising two of the NeoTCRs (equal amounts of each of the two NeoTCR cells). As shown, while the 2 NeoTCR Products exhibited killing, none of the 2 NeoTCR combinations exhibited the same degree of killing as the 3 NeoTCR Product as shown in the top left graph. FIG. 8E shows two charts: the first chart shows that the 3 NeoTCRs used in the NeoTCR Products shows in FIGS. 7A-7D have different neoepitopes, that there is a diversity of HLAs, and difference in physiological properties of the NeoTCRs; the second chart shows that each of the NeoTCRs used in the NeoTCR Products shown in FIGS. 8A-8D have different neoepitopes, different HLAs, and difference in physiological properties of the NeoTCRs.

FIGS. 9A-9C show that the levels of HLA expression of the M490 cell line can be enhanced to better mimic tumor cell HLA expression by pretreatment with IFNγ. FIG. 9A shows the endogenous HLA expression on M490 cells without IFNγ pretreatment. FIG. 9B shows that IFNγ pretreatment upregulates HLA expression (FMO is the control, no stain). FIG. 9C presents RNAseq data which shows a heat map plot of the upregulation of HLA Class I after IFNγ pretreatment in both M486 and M490 cells. In these experiments, the M490 cell line was engineered to express the NeoTCR. Accordingly, FIGS. 9B and 9C shows that IFNγ pretreatment can be used to induce a more natural phenotype of cancer cells by upregulating HLA class 1 and neoantigen presentation.

FIGS. 10A and 10B show that although IFNγ pretreatment has a cytostatic effect on the M490 cell line (FIG. 10A), it does not affect cell viability (FIG. 10B).

FIGS. 11A and 11B show that NeoTCR Products exhibit a higher degree of cytotoxicity when pretreated with IFNγ. FIG. 11A shows a tumor killing assay using 1 NeoTCR Products (and the M490 cell line that is not transfected with a NeoTCR as a control). As shown certain of the 1 NeoTCR Products show no killing (NeoTCR 423, NeoTCR 418, and NeoTCR 433 show no killing in line with the control M490 cells that too show no killing). FIG. 11B shows the same tumor killing assay as shown in FIG. 11A but with IFNγ pretreatment. This shows that IFNγ pretreatment increases the killing ability of NeoTCR Products.

FIG. 12 shows the experimental setup for the peptide pulsing experiments. As shown, tumor cells (melanoma cells cultured from a patient biopsy) are pulsed a neopeptide that is specific for the neoepitope expressed on the NeoTCR Product (i.e., a cognate neopeptide to the NeoTCR). Killing assays were performed with and without IFNγ pretreatment and killing was measured by an IncuCyte assay. In these experiments, the P:T ratio (NeoTCR cell:Tumor cell ratio) was 10:1.

FIGS. 13A and 13B show that the NeoTCR Product with NeoTCR 409 (FIG. 13A) and NeoTCR Product with NeoTCR 422 (FIG. 13B), both of which NeoTCRs are HLA-A02 restricted, show superior cytotoxicity of autologous tumor cells after peptide pulsing with the cognate neopeptide.

FIGS. 14A-14C show that the NeoTCR Product with NeoTCR 418 (FIG. 14A), NeoTCR Product with NeoTCR 433 (FIG. 14B), and NeoTCR Product with NeoTCR 423 (FIG. 14C), each of which NeoTCRs are HLA-B15 restricted, show autologous tumor cell killing with IFNγ pretreatment and peptide pulsing with the cognate neopeptide. As opposed to the HLA-A02 restricted NeoTCR Products shown in FIGS. 13A and 13B, the HLA-B15 restricted NeoTCR Products shown in FIGS. 14A-14C show significant tumor cell killing increase when pretreated with IFNγ demonstrating that HLA presentation is dependent on the HLA.

FIG. 15 shows the experimental design of a NeoTCR priming experiment. Specifically, the experiment was designed to test the hypothesis that two NeoTCRs can synergistically work together to promoted tumor cell killing. In this experiment two NeoTCR Products were made, each product specific to one of two neoantigens expressed on the tumor cells (melanoma cells cultured from a patient biopsy). The tumor cells were first cultured for 20 hrs with the first NeoTCR Product and then cultures with or without the second NeoTCR Product.

FIG. 16 shows the results of a priming experiment. The two NeoTCR Products used were: 1) NeoTCR Product with NeoTCR 422 (Product 1), and 2) NeoTCR Product with NeoTCR 421 (Product 2). The hypothesis was that if the tumor cells are primed with a first NeoTCR Product, it will induce an apoptotic signal and make the tumor cells more susceptible to subsequent killing by a second NeoTCR Product. Tumor cells were primed with either primed with Product 1 or Product 2 for 20 hrs. The priming media containing Product 1 or Product 2 was removed and then the cells were either treated with RPMI media alone or with the other Product. The P:T ratio used in this experiment was 30:1. As shown, both NeoTCR Products exhibited enhanced killing following pretreatment with the other NeoTCR Product.

FIGS. 17A and 17B show that priming tumor cells with a first NeoTCR Product enhances the killing of a second NeoTCR Product. FIG. 17A shows that the NeoTCR Product expressing NeoTCR 429 exhibits enhanced killing following the priming of the tumor cells by the NeoTCR Product expressing NeoTCR 422. FIG. 17A shows that the NeoTCR Product expressing NeoTCR 429 exhibits enhanced killing following the priming of the tumor cells by the NeoTCR Product expressing NeoTCR 421. In both experiments, while the priming media does induce some killing of the tumor cells, the priming plus the second NeoTCR Product induces significantly more killing.

FIGS. 18A-18D shows an unexpected synergy between different NeoTCR cells. FIG. 18A shows while each of NeoTCR Product expressing NeoTCR 422 (NeoTCR Product 1), NeoTCR Product expressing NeoTCR 429 (NeoTCR Product 2), and NeoTCR Product expressing NeoTCR 421 (NeoTCR Product 3) exhibit some degree of killing of tumor cells, NeoTCR Product 4 (comprising NeoTCR cells expressing either NeoTCR 422, NeoTCR 429, or NeoTCR 421; each NeoTCR cell comprising ⅓ of the total NeoTCR cells in NeoTCR Product 4 as described in FIG. 5) exhibits significantly more tumor cell killing demonstrating the synergistic effect of 3 NeoTCRs in a NeoTCR Product. In order to understand whether a 3 NeoTCR Product was better than a 2 NeoTCR Product, 2 NeoTCR Products were made using each combination of NeoTCR 422, NeoTCR 429, or NeoTCR 421 (FIGS. 18B-18D). These experiments were performed using each of the 3 NeoTCR combinations presented in FIG. 6B and the unexpected synergistic effect of 3 NeoTCRs v. 1 or 2 NeoTCRs was shown in each experiment.

FIG. 19 show that 1 NeoTCR Products to not effectively kill autologous tumor cells at a limited 10:1 P:T ratio and also shows the efficacy of CD8 independent v. CD8 dependent NeoTCRs.

FIG. 20 shows the synergistic effect of the cytotoxicity of 3 NeoTCR Products composed of NeoTCRs isolated from a patient against the autologous tumor cell line. The combination of 3 NeoTCRs targeted different neoepitopes and different HLAs. The NeoTCRs when tested as single NeoTCR are not able to control the tumor cell line. When tested in combination, the NeoTCRs showed synergistic effect and were able to control the tumor cell line as illustrated by the decrease in the percentage of red fluorescent cells. No effect on tumor cell growth was observed with RPMI or the Neo12 TCR expressing T cells (the controls). The flow plots on the right show the dextramer and 2A staining on the CD4 and CD8 cells. The 3 NeoTCRs tested, TCR406, TCR418 and TCR429 are CD8 dependent NeoTCRs. In these NeoTCRs the percentage of dextramer binding in CD4 cells is less than 50% of the dextramer binding in CD8 cells.

### DETAILED DESCRIPTION OF THE INVENTION

The present disclosure provides compositions with enhanced activity and efficacy for immunotherapy. The present disclosure is based, in part, on the surprising results showing that modified cells targeting tumor antigens (e.g., a neoantigen) and showing no or negligible cytotoxic activity when alone have an increased activity (e.g., cytotoxicity, cell proliferation, and/or cell persistence) when combined. The present disclosure also provides methods for producing the plurality of cells and compositions disclosed herein, and methods of using such cells for treating and/or preventing cancer.

Non-limiting embodiments of the present disclosure are described by the present description and examples. For purposes of clarity of disclosure and not by way of limitation, the detailed description is divided into the following subsections:

1. Definitions;

2. NeoTCR Cells and NeoTCR Products;

3. Therapeutic Composition and Methods of Manufacturing;

4. Therapeutic Methods;

5. Pharmaceutical Formulations;

6. Articles of Manufacture;

7. Genome Editing Methods;

8. Homology Recombination Templates; and

9. Kits.

### 1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

It is understood that aspects and embodiments of the invention described herein include “comprising,” “consisting,” and “consisting essentially of” aspects and embodiments. The terms “comprises” and “comprising” are intended to have the broad meaning ascribed to them in U.S. Patent Law and can mean “includes”, “including” and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

“Acetaminophen” as used herein means acetaminophen and any other branded or time-released alternatives of the same and/or therapeutically similar agents such as paracetamol.

“Administering” as used herein mean a method of giving a dosage of a compound (e.g., a NeoTCR Product, a combination agent, or other agent described herein) or a composition (e.g., a pharmaceutical formation of a NeoTCR Product, a combination agent, or other agent described herein) to a subject. The compositions utilized in the methods described herein can be administered, for example, intravenously, intraperitoneally, intravesicularly, intratumorally and subcutaneously. Additional modes of administration include but are not limited to intrathecally, lymph node injection, intramuscularly, intradermally, intraarterially, intralesionally, intracranially, intrapleurally, intratracheally, intravitreally, peritoneally, and intraumbilically. Any administration disclosed herein can be performed by injection, by infusion, by continuous infusion, by localized perfusion bathing target cells directly, by catheter, or by lavage. The method of administration can vary depending on various factors (e.g., the severity of the condition, disease, or disorder being treated, and the type of cancer or proliferative disorder being treated).

“Antibody” as used herein is used in the broadest sense and encompasses various antibody structures, including but not limited to monoclonal antibodies, polyclonal antibodies, multispecific antibodies (e.g., bispecific and tri-specific antibodies), and antibody fragments (e.g., bis-Fabs) so long as they exhibit the desired antigen-binding activity. “Antibody Fragment” as used herein refers to a molecule other than an intact antibody that comprises a portion of an intact antibody that binds the antigen to which the intact antibody binds. Examples of antibody fragments include but are not limited to bis-Fabs; Fv; Fab; Fab, Fab′-SH; F(ab′)2; diabodies; linear antibodies; single-chain antibody molecules (e.g., scFv); and multispecific antibodies formed from antibody fragments.

The terms “Cancer” and “Tumor” are used interchangeably herein. As used herein, the terms “Cancer” or “Tumor” refer to all neoplastic cell growth and proliferation, whether malignant or benign, and all pre-cancerous and cancerous cells and tissues. The terms are further used to to refer to or describe the physiological condition in mammals that is typically characterized by unregulated cell growth/proliferation. Examples of cancer include, but are not limited to, those described herein. The terms “Cancer” or “Tumor” and “Proliferative Disorder” are not mutually exclusive as used herein.

A “chemotherapeutic agent” as used herein refers to a chemical compound useful in the treatment of cancer. Examples of chemotherapeutic agents include alkylating agents such as thiotepa, cyclosphosphamide (CYTOXAN®), temozolomide (Methazolastone®, Temodar®), treosultan, and bendamustine hydrochloride (Treanda®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), irinotecan liposome injection (Onivyde®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine, cladribine (Leustat®) and nelarabine (Arranon®); pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), cabazitaxel (Jevtana®), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vincristine sulfate liposome (Marqibo®), vindesine (ELDISINE®, FILDESIN®), vinflunine (Javlor®) and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); sorafenib (e.g. Nexavar®); SU-11248 (sunitinib, SUTENT®, Pfizer); perifosine, COX-2 inhibitor (e.g. celecoxib or etoricoxib), proteosome inhibitor (e.g. PS341) such as carfilzomib (Kyprolis®) and ixazomib citrate (Ninlaro®); bortezomib (VELCADE®); CCI-779; tipifarnib (R11577); orafenib, ABT510; Bcl-2 inhibitor such as oblimersen sodium (GENASENSE®) and venetoclax (Venclexta®); pixantrone; EGFR inhibitors (see definition below) such as gefitinib (Iressa®); tyrosine kinase inhibitors (see definition below) such as bosutinib (Bosulif®), cabozantinib-s-malate (Cabometyx®, Cometriq®), afatinib dimaleate (Gilotrif®), imatinib mesylate (Gleevec®), ponatinib hydrochloride (Iclusig®); axitinib (Inlyta®), ibrutinib (Imbruvica®), sorafenib tosylate (Nexavar®), dasatinib (Sprycel®), osimertinib (Tagrisso®), erlotinib hydrochloride (Tarceva®), nilotinib (Tasigna®), lapatinib ditosylate (Tykerb®), crizotinib (Xalkori®) and pazopanib hydrochloride (Votrient®); serine-threonine kinase inhibitors such as rapamycin (sirolimus, RAPAMUNE®), everolimus (Afinitor®), and temsirolimus (Torisel®); farnesyltransferase inhibitors such as lonafarnib (SCH 6636, SARASAR™); NK1 receptor antagonist such as netupitant, and rolapitant hydrochloride (Varubi®); imiquimod (Aldara®); anaplastic lymphoma kinase (ALK) inhibitor such as alectinib (Alecensa®) and ceritinib (Zykadia®); histone deacetylase inhibitors such as belinostat (Beleodaq®) and vorinostat (Zolinza®); purine nucleoside antimetabolite such as clofarabine; mitogen-activated protein kinase kinase (MEK) inhibitors such as cobimetinib (Cotellic®) and trametinib (Mekinist®); nucleic acid synthesis inhibitors such as decitabine (Dacogen®); Hedgehog signaling pathway inhibitor such as vismodegib (Erivedge®) and sonidegib (Odomzo®); histone deacetylase inhibitor such as panobinostat (Farydak®); antifolate such as pralatrexate (Folotyn®), raltitrexed, and pemetrexed disodium (Alimta®); mitotic inhibitor such as eribulin mesylate (Halaven®); inhibitor of the cyclin-dependent kinases such as palbociclib (Ibrance®); depsipeptide such as romidepsin (Istodax®); epothilone B analog such as ixabepilone (Ixempra®); Janus kinase inhibitor such as ruxolitinib phosphate (Jakafi®); multiple kinase inhibitor such as lenvatinib mesylate (Lenvima®), vandetanib (Caprelsa®), regorafenib (Stivarga®), nintedanib (Vargatef®); nucleoside analog such as trifluridine; thymidine phosphorylase inhibitor such as tipiracil hydrochloride; PARP inhibitor such as olaparib (Lynparza®); thalidomide (Stivarga®, Thalomid®) and its derivative such as pomalidomide (Pomalyst®) and lenalidomide (Revlimid®); synthetic corticosteroid such as prednisone; analog of somatostatin such as lanreotide acetate (Somatuline®); protein translation inhibitor such as omacetaxine mepesuccinate (Synribo®); inhibitor of the associated enzyme B-Raf such as dabrafenib (Tafinlar®) and vemurafenib (Zelboraf®); arsenic trioxide (Trisenox®); uridine triacetate (Vistogard®); radium 223 dichloride (Xofigo®); trabectedin (Yondelis), phosphoinositide 3-kinase inhibitor such as idelalisib (Zydelig®); milfamurtide (Mepact®); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; gonadotropin-releasing hormone (GnRH) antagonist such as degarelix, leuproprelin, and triptorelin; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrozole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releasing hormone agonists, including leuprolide (LUPRON® and ELIGARD®), goserelin, buserelin, histrelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; dexamethasone; onapristone; anti-progesterones; estrogen receptor down-regulators (ERDs); anti-androgens such as flutamide, nilutamide (e.g. Nilandron®), abiraterone acetate (Zytiga®), cyproterone acetate (Cyprostat®), enzalutamide (Xtandi®) and bicalutamide; and pharmaceutically acceptable salts, acids or derivatives of any of the above; as well as combinations of two or more of the above.

“Combination Agent” as used herein means any other active agent that can be administered simultaneously or consecutively in combination with one or more NeoTCR Products.

A “Complete Response” as used herein means the disappearance of all signs of cancer in response to treatment.

“Cytotoxic agent” as used herein means a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents such as large molecules (e.g., antibodies, peptides, and proteins such as cytokines), cellular therapies, and cancer vaccines.

“Delaying Progression” or “Delayed Progression” as used herein means to defer, hinder, slow, retard, stabilize, and/or postpone development of a cell proliferative disorder such as cancer. This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “effective amount” or “therapeutically effective amount” of a NeoTCR Product (or a combination comprising such a NeoTCR Product) as disclosed herein is an amount sufficient to carry out a specifically stated purpose. An “effective amount” or “therapeutically effective amount” refers to an amount of a NeoTCR Product or combination comprising such a NeoTCR Product as disclosed herein, effective to “treat” a disease or disorder in a mammal (e.g., a human patient). In the case of cancer or other proliferative disorder (including but not limited to one of the cancer types described herein), the therapeutically effective amount of a NeoTCR Product or combination comprising such a NeoTCR Product refers to 1) the amount necessary to reduce, stop or prevent the spread of such cancer, 2) the amount necessary to reduce, stop or prevent one or more symptoms of such cancer, and/or 3) the amount necessary to eradicate all or a substantial amount of the cancer. In certain embodiments, an effective amount of a NeoTCR Product will result in a partial response. In certain embodiments, an effective amount of a NeoTCR Product will result in a complete response.

In certain embodiments, an “effective amount” of a NeoTCR Product is at least the minimum amount of the NeoTCR Product required to achieve the desired therapeutic or prophylactic result, such as a measurable improvement or prevention of a particular disorder (e.g., a proliferative disorder, e.g., cancer). An effective amount herein may vary according to factors such as the disease state, age, sex, and weight of the patient, and the ability of the NeoTCR Product to elicit a desired response in the patient. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. An effective amount the NeoTCR Product is an amount sufficient to accomplish prophylactic or therapeutic treatment either directly or indirectly. As is understood in the clinical context, an effective amount of a NeoTCR Product may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

“ELISA” as used herein means enzyme-linked immunosorbent assay.

“FISH” as used herein means fluorenone in situ hybridization.

“Gene Circuit” as used herein means gene constructs that interact to execute a user-defined biological program. In certain embodiments, gene circuits are an application of synthetic biology wherein biological parts inside a cell are designed to perform functions (i.e., mimicking those observed in electronic circuits). The applications of gene circuits include but are not limited to 1) inducing production of a compound or molecule (e.g., a protein, a polypeptide, a cytokine, an antibody, or any other compound or molecule that a cell can naturally produce or be engineered to produce) that is either not naturally produced by a cell or to increase production of a compound or molecule that is naturally produced by a cell, 2) adding a new element to a cell (e.g., a protein, a polypeptide, a cytokine, an antibody, or any other compound or molecule that a cell can naturally produce or be engineered to produce), and 3) implementing new systems within a cell.

“IL-2 Agent” as used herein means any IL-2 cytokine, molecule comprising a IL-2 cytokine or a functional fragment thereof, a derivation of an IL-2 cytokine, a modified IL-2 cytokine, a pegylated IL-2 cytokine, an antibody fused to an IL-2 cytokine or a function fragment thereof, an Fc fused to an IL-2 cytokine or a function fragment thereof, or another antibody fragment fused to an IL-2 cytokine or a function fragment thereof.

“IL-7 Agent” as used herein means any IL-7 cytokine, molecule comprising a IL-7 cytokine or a functional fragment thereof, a derivation of an IL-7 cytokine, a modified IL-7 cytokine, a pegylated IL-7 cytokine, an antibody fused to an IL-7 cytokine or a function fragment thereof, an Fc fused to an IL-7 cytokine or a function fragment thereof, or another antibody fragment fused to an IL-7 cytokine or a function fragment thereof.

“IL-10 Agent” as used herein means any IL-10 cytokine, molecule comprising a IL-10 cytokine or a functional fragment thereof, a derivation of an IL-10 cytokine, a modified IL-10 cytokine, a pegylated IL-10 cytokine, an antibody fused to an IL-10 cytokine or a function fragment thereof, an Fc fused to an IL-10 cytokine or a function fragment thereof, or another antibody fragment fused to an IL-10 cytokine or a function fragment thereof.

“IL-12 Agent” as used herein means any IL-12 cytokine, molecule comprising a IL-12 cytokine or a functional fragment thereof, a derivation of an IL-12 cytokine, a modified IL-12 cytokine, a pegylated IL-12 cytokine, an antibody fused to an IL-12 cytokine or a function fragment thereof, an Fc fused to an IL-12 cytokine or a function fragment thereof, or another antibody fragment fused to an IL-12 cytokine or a function fragment thereof.

“IL-15 Agent” as used herein means any IL-15 cytokine, molecule comprising a IL-15 cytokine or a functional fragment thereof, a derivation of an IL-15 cytokine, a modified IL-15 cytokine, a pegylated IL-15 cytokine, an antibody fused to an IL-15 cytokine or a function fragment thereof, an Fc fused to an IL-15 cytokine or a function fragment thereof, or another antibody fragment fused to an IL-15 cytokine or a function fragment thereof.

“IL-18 Agent” as used herein means any IL-18 cytokine, molecule comprising a IL-18 cytokine or a functional fragment thereof, a derivation of an IL-18 cytokine, a modified IL-18 cytokine, a pegylated IL-18 cytokine, an antibody fused to an IL-18 cytokine or a function fragment thereof, an Fc fused to an IL-18 cytokine or a function fragment thereof, or another antibody fragment fused to an IL-18 cytokine or a function fragment thereof.

“IL-21 Agent” as used herein means any IL-21 cytokine, molecule comprising a IL-21 cytokine or a functional fragment thereof, a derivation of an IL-21 cytokine, a modified IL-21 cytokine, a pegylated IL-21 cytokine, an antibody fused to an IL-21 cytokine or a function fragment thereof, an Fc fused to an IL-21 cytokine or a function fragment thereof, or another antibody fragment fused to an IL-21 cytokine or a function fragment thereof.

“Label” or “Package Insert” is used to refer to instructions customarily included in commercial packages of therapeutic products, that contain information about the indications, usage, dosage, administration, combination therapy, contraindications and/or warnings concerning the use of such therapeutic products. Unless specified herein, a “Label” or “Package Insert” refers to the label or package insert of the NeoTCR Product.

As used herein, the terms “neoantigen”, “neoepitope” or “neoE” refer to a newly formed antigenic determinant that arises, e.g., from a somatic mutation(s) and is recognized as “non-self.” A mutation giving rise to a “neoantigen”, “neoepitope” or “neoE” can include a frameshift or non-frameshift indel, missense or nonsense substitution, splice site alteration (e.g., alternatively spliced transcripts), genomic rearrangement or gene fusion, any genomic or expression alterations, or any post-translational modifications.

“NeoTCR” and “TCR” are used interchangeably and as used herein mean a neoepitope-specific T cell receptor.

“NeoTCR cells” or “NeoTCR Cells” as used herein means one or more cells precision engineered to express one or more NeoTCRs. In certain embodiments, the cells are T cells. In certain embodiments, the T cells are CD8+ and CD4+ T cells. In certain embodiments, the CD8+ and CD4+ T cells (i.e., the NeoTCR cells) are autologous cells from the patient for whom a NeoTCR Product will be administered.

“NeoTCR cell population” as used herein means a population of NeoTCR Cells wherein each of the cells in the population were precision engineered to express the same NeoTCR.

“NeoTCR Product” or “TCR Product” as used herein means a pharmaceutical formulation comprising one or more NeoTCRs. NeoTCR Product comprises autologous precision genome-engineered CD8+ and CD4+ T cells. For example, using a targeted DNA-mediated non-viral precision genome engineering approach, expression of the endogenous TCR is eliminated and replaced by a patient-specific NeoTCR isolated from peripheral CD8+T cells targeting the tumor-exclusive neoepitope. In certain embodiments, the resulting engineered CD8+ and CD4+ T cells express NeoTCRs on their surface of native sequence, native expression levels, and native TCR function. The sequences of the NeoTCR external binding domain and cytoplasmic signaling domains are unmodified from the TCR isolated from native CD8+ T cells. Regulation of the NeoTCR gene expression is driven by the native endogenous TCR promoter positioned upstream of where the NeoTCR gene cassette is integrated into the genome. Through this approach, native levels of NeoTCR expression are observed in unstimulated and antigen-activated T cell states.

The NeoTCR Product manufactured for each patient represents a defined dose of autologous CD8+ and CD4+ T cells that are precision genome engineered to express a single neoE-specific TCR cloned from neoE-specific CD8+ T cells individually isolated from the peripheral blood of that same patient.

In certain embodiments, the NeoTCR Product comprises a plurality of different populations of NeoTCR Cells, each population targeting a different neoantigen. In certain embodiments, the NeoTCR Product comprises one population of NeoTCR Cells (i.e., all of the NeoTCR Cells of the population target the same neoantigen). In certain embodiments, the NeoTCR Product comprises two populations of NeoTCR Cells (i.e., all of the NeoTCR Cells of the first NeoTCR Cell population target one neoantigen and all of the NeoTCR Cells of the second NeoTCR Cell population target a second neoantigen). In certain embodiments, the NeoTCR Product comprises three populations of NeoTCR Cells (i.e., all of the NeoTCR Cells of the first NeoTCR Cell population target one neoantigen, all of the NeoTCR Cells of the second NeoTCR Cell population target a second neoantigen, all of the NeoTCR Cells of the third NeoTCR Cell population target a third neoantigen). In certain embodiments, the NeoTCR Product comprises two or more NeoTCR Cells. In certain embodiments, the NeoTCR Product comprises three or more NeoTCR Cells. In certain embodiments, the NeoTCR Product comprises five or more NeoTCR Cells. In certain embodiments, the number of each NeoTCR Cells of each NeoTCR Cell population in a NeoTCR Product with two or more populations of NeoTCR Cells are approximately equal to each other (i.e., a NeoTCR Product with three NeoTCR Cell populations comprise ⅓ a first NeoTCR Cell population, ⅓ a second NeoTCR population, and ⅓ a third NeoTCR population).

A NeoTCR Product refers to all NeoTCR Products including but not limited to One NeoTCR Products, Two NeoTCR Products, and Three NeoTCR Products as defined below.

“One NeoTCR Product” or “1 NeoTCR Product” as used herein refers to a NeoTCR Product comprising cells that only express a single NeoTCR.

“Two NeoTCR Product” or “2 NeoTCR Product” as used herein refers to a NeoTCR Product comprising two populations of cells: one population that expresses a first NeoTCR and a second population that express a second NeoTCR. In certain embodiments, the 2 NeoTCR Product has an approximately equal number of each of the two cell populations. In certain embodiments, the 2 NeoTCR Product does not have an approximately equal number of each of the two cell populations. In certain embodiments, each population of cells can be considered a NeoTCR Product and the combination of each of the two cell populations is also considered a NeoTCR Product.

“Three NeoTCR Product” or “3 NeoTCR Product” as used herein refers to a NeoTCR Product comprising three populations of cells: one population that expresses a first NeoTCR, a second population that express a second NeoTCR, and a third population that expresses a third NeoTCR. In certain embodiments, the 3 NeoTCR Product has an approximately equal number of each of the three cell populations. In certain embodiments, the 3 NeoTCR Product does not have an approximately equal number of each of the three cell populations. In certain embodiments, each population of cells can be considered a NeoTCR Product and the combination of each of the three cell populations is also considered a NeoTCR Product.

“2A” and “2A peptide” are used interchangeably herein and mean a class of 18-22 amino acid long, viral, self-cleaving peptides that are able to mediate cleavage of peptides during translation in eukaryotic cells. Four well-known members of the 2A peptide class are T2A, P2A, E2A, and F2A. The T2A peptide was first identified in the Thosea asigna virus 2A. The P2A peptide was first identified in the porcine teschovirus-1 2A. The E2A peptide was first identified in the equine rhinitis A virus. The F2A peptide was first identified in the foot-and-mouth disease virus.

“In combination with,” as used herein, means that a plurality of cells disclosed herein, and one or more agents, e.g., a PD-1 axis binding agent, are administered to a subject as part of a treatment regimen or plan.

“Partial Response” as used herein means a decrease in the size of a tumor, or in the extent of cancer in the body, in response to treatment.

“PD” as used herein means pharmacodynamics.

The term “PD-1 axis binding agent” refers to a molecule that inhibits the interaction of a PD-1 axis binding partner with either one or more of its binding partner, so as to remove T cell dysfunction resulting from signaling on the PD-1 signaling-axis with a result being to restore or enhance T cell function (e.g., proliferation, cytokine production, target cell killing). As used herein, a PD-1 axis binding agent includes a PD-1 binding agent, a PD-L1 binding agent and a PD-L2 binding agent.

The term “PD-1 binding agent” refers to a molecule that decreases, blocks, inhibits, abrogates or interferes with signal transduction resulting from the interaction of PD-1 with one or more of its binding partners, such as PD-L1 and/or PD-L2. In some embodiments, the PD-1 binding agent is a molecule that inhibits the binding of PD-1 to one or more of its binding partners. In a specific embodiment, the PD-1 binding agent inhibits the binding of PD-1 to PD-L1 and/or PD-L2. For example, PD-1 binding agent include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding agent reduces the negative co-stimulatory signal mediated by or through cell surface proteins expressed on T lymphocytes mediated signaling through PD-1 so as render a dysfunctional T cell less dysfunctional (e.g., enhancing effector responses to antigen recognition). In certain embodiments, the PD-1 binding agent is an anti-PD-1 antibody. In certain embodiments, the PD-1 binding agent is MDX-1106 (nivolumab). In certain embodiments, the PD-1 binding agent is MK-3475 (pembrolizumab). In certain embodiments, the PD-1 binding agent is cemiplimab. In certain embodiments, the PD-1 binding agent is JTX-4014. In certain embodiments, the PD-1 binding agent is spartalizumab. In certain embodiments, the PD-1 binding agent is sintilimab. In certain embodiments, the PD-1 binding agent is tislelizumab. In certain embodiments, the PD-1 binding agent is toripalimab. In certain embodiments, the PD-1 binding agent is dostarlimab. In certain embodiments, the PD-1 binding agent is MGA012. In certain embodiments, the PD-1 binding agent is AMP-514. In certain embodiments, the PD-1 binding agent is CT-011 (pidilizumab). In certain embodiments, the PD-1 binding agent is AMP-224. In certain embodiments, the PD-1 binding agent is MED1-0680. In certain embodiments, the PD-1 binding agent is PDR001. In certain embodiments, the PD-1 binding agent is REGN2810. In certain embodiments, the PD-1 binding agent is BGB-108.

The term “PD-L1 binding agent” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding agent is a molecule that inhibits the binding of PD-L1 to its binding partners. In a specific embodiment, the PD-L1 binding agent inhibits binding of PD-L1 to PD-1 and/or B7-1. In some embodiments, the PD-L1 binding agent include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding agent reduces the negative co-stimulatory signal mediated by or through cell surface proteins expressed on T lymphocytes mediated signaling through PD-L1 so as to render a dysfunctional T cell less dysfunctional (e.g., enhancing effector responses to antigen recognition). In some embodiments, a PD-L1 binding antagonist is an anti-PD-L1 antibody. In still another specific embodiment, an anti-PD-L1 antibody is MPDL3280A (atezolizumab, marketed as TECENTRIQ™ with a WHO Drug Information (International Nonproprietary Names for Pharmaceutical Substances), Recommended INN: List 74, Vol. 29, No. 3, 2015 (see page 387)). In a specific embodiment, an anti-PD-L1 antibody is YW243.55.S70. In another specific embodiment, an anti-PD-L1 antibody is MDX-1105. In another specific embodiment, an anti PD-L1 antibody is MSB0015718C. In still another specific embodiment, an anti-PD-L1 antibody is MED14736.

The term “PD-L2 binding agent” refers to a molecule that decreases, blocks, inhibits, abrogates or interferes with signal transduction resulting from the interaction of PD-L2 with either one or more of its binding partners, such as PD-1. In some embodiments, a PD-L2 binding agent is a molecule that inhibits the binding of PD-L2 to one or more of its binding partners. In a specific embodiment, the PD-L2 binding agent inhibits binding of PD-L2 to PD-1. In some embodiments, the PD-L2 agent include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding agent reduces the negative co-stimulatory signal mediated by or through cell surface proteins expressed on T lymphocytes mediated signaling through PD-L2 so as render a dysfunctional T cell less dysfunctional (e.g., enhancing effector responses to antigen recognition). In some embodiments, a PD-L2 binding agent is an immunoadhesin.

“Pharmaceutical Formulation” refers to a preparation which is in such form as to permit the biological activity of an active ingredient contained therein to be effective, and which contains no additional components which are unacceptably toxic to a subject to which the formulation would be administered. For clarity, DMSO at quantities used in a NeoTCR Product are not considered unacceptably toxic.

“Proliferative disorder” as used herein means disorders that are associated with some degree of abnormal cell proliferation. In certain embodiments, the proliferative disorder is cancer.

A “subject,” “patient,” or an “individual” for purposes of treatment refers to any animal classified as a mammal, including humans, domestic and farm animals, and zoo, sports, or pet animals, such as dogs, horses, cats, cows, etc. Preferably, the mammal is human.

“TCR” as used herein means T cell receptor.

“TLA” as used herein means targeted locus amplification.

“Treat,” “Treatment,” and “treating” are used interchangeably and as used herein mean obtaining beneficial or desired results including clinical results. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the NeoTCR Product of the invention are used to delay development of a proliferative disorder (e.g., cancer) or to slow the progression of such disease.

“Truncal” or “Truncal Mutation” as used herein means the ancestral mutations in the trunk of the phylogenetic tree that are shared by all clones of the tumor, while “subclonal” by contrast refers to mutations in a lineage that has diverged from the trunk. Every patient's tumors have a unique set of shared mutations to indicate a common evolutionary origin. Accordingly, as used herein, truncal mutations of a patient refer to the shared mutations across all tumors of the same lineage in a patient.

The terms “polynucleotide” and “nucleic acid molecule,” as used herein, refer to a single or double-stranded covalently-linked sequence of nucleotides in which the 3′ and 5′ ends on each nucleotide are joined by phosphodiester bonds. The nucleic acid molecule can include deoxyribonucleotide bases or ribonucleotide bases, and can be manufactured synthetically in vitro or isolated from natural sources.

The terms “polypeptide,” “peptide,” “amino acid sequence” and “protein,” used interchangeably herein, refer to a molecule formed from the linking of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis or enzymatic synthesis. The terms can apply to amino acid polymers in which one or more amino acid residue is an artificial chemical mimetic of a corresponding naturally occurring amino acid, as well as to naturally occurring amino acid polymers and non-naturally occurring amino acid polymers.

The term “endogenous” as used herein refers to a nucleic acid molecule or polypeptide that is normally expressed in a cell or tissue.

The term “exogenous” as used herein refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

The term “liquid cancer” as used herein refers to cancer cells that are present in body fluids, such as blood, lymph and bone marrow. In certain non-limiting embodiments, for example, liquid cancers include follicular lymphoma, leukemia, multiple myeloma, myelodysplastic syndrome (MDS), and acute myeloid leukemia (AML).

As used herein, the term “solid cancer” refers to cancer cells that are arising from tissues that do not include fluid areas. In certain non-limiting embodiments, for example, solid cancers include melanoma, thoracic cancer, lung cancer, ovarian cancer, breast cancer, pancreatic cancer, head and neck cancer, prostate cancer, gynecological cancer, central nervous system cancer, cutaneous cancer, HPV+ cancer, esophageal cancer, thyroid cancer, gastric cancer, hepatocellular cancer, cholangiocarcinoma, renal cell cancer, bladder cancer, testicular cancer, sarcoma, and colorectal cancer.

“Young” or “Younger” or “Young T cell” as it relates to T cells means memory stem cells (T_{MSC}) and central memory cells (T_{CM}). These cells have T cell proliferation upon specific activation and are competent for multiple cell divisions. They also have the ability to engraft after re-infusion, to rapidly differentiate into effector T cells upon exposure to their cognate antigen and target and kill tumor cells, as well as to persist for ongoing cancer surveillance and control.

### 2. NeoTCR Cells and NeoTCR Products

The present disclosure provides for a plurality of genome-engineered cells, wherein each cell targets a tumor antigen (e.g., a neoantigen). In certain embodiments, the genome-engineered cells exhibit improved activity (e.g., cytotoxicity) when combined with other genome-engineered cells targeting a different tumor antigen. In certain embodiments, the plurality of genome-engineered cells exhibits a synergistic effect. A synergistic effect is achieved when active ingredients used together (e.g., two or more NeoTCR cells) show a biological response that is greater than the sum of the effects resulting from using the active ingredients separately.

In certain embodiments, using the gene editing technology and NeoTCR isolation technology described in International Patent Application No. PCT/US2020/017887 and International Patent Application No. PCT/US2019/025415, which are incorporated herein in their entireties, NeoTCRs are cloned in autologous CD8+ and CD4+ T cells from the same cancer patient by precision genome engineering such that the autologous CD8+ and CD4+ T cells express the NeoTCR. In certain embodiments, the NeoTCR is tumor specific. In certain embodiments, the NeoTCR is identified in cancer patients. In certain embodiments, a polynucleotide encoding the NeoTCR is cloned and expressed in immune cells. In certain embodiments, the cells are T cells. In certain embodiments, the T cells are young T cells.

In certain embodiments, the plurality of cells is manufactured starting from T cells of a cancer patient. In certain embodiments, each cell of the plurality of cells has comparable gene editing efficiency. In certain embodiments, each cell of the plurality of cells has comparable activity (e.g., antigen-dependent cytotoxicity, proliferation, cytokine production). In certain embodiments, the plurality of cells shows a safe tolerability profile.

In certain embodiments, the gene-editing of the plurality of cells comprises electroporation of the cells with a ribonucleoprotein (RNP) complex. In certain embodiments, the RNP complex comprises a guide RNA. In certain embodiments, the RNP complex comprises a nuclease. In certain embodiments, the nuclease is a Cas9 nuclease. In certain embodiments, the RNP complex targets endogenous loci. In certain embodiments, the RNP complex targets a TCR locus. In certain embodiments, the RNP complex targets a TRAC locus. In certain embodiments, the RNP complex targets a TRBC locus. In certain embodiments, the RNP complex target TRAC and TRBC loci.

In certain embodiments, the present disclosure comprises methods for determining the safety profile of the plurality of cells disclosed herein. In certain non-limiting embodiments, for example, the safety profile can be assessed by detecting genomic instability (e.g., by performing targeted locus amplification (TLA) or standard FISH cytogenetics), or by detecting off-target integration (e.g., by Guide-Seq analysis).

In certain non-limiting embodiments, the present disclosure comprises multiple NeoTCR Cells targeting different tumor antigens into a NeoTCR Product. In certain embodiments, a first NeoTCR Cell of the NeoTCR Product targets a first tumor antigen and exhibits improved activity (e.g., cytotoxicity) when combined with a second NeoTCR Cell targeting a second tumor antigen. In certain embodiments, the multiple NeoTCR Cells exhibit a synergistic effect.

In certain embodiments, the NeoTCR Products are manufactured starting from T cells of a cancer patient. In certain embodiments, the multiple NeoTCR Cells of the NeoTCR Product have comparable gene editing efficiency. In certain embodiments, the multiple NeoTCR Cells of the NeoTCR Product have comparable activity (e.g., antigen-dependent cytotoxicity, proliferation, cytokine production). In certain embodiments, the NeoTCR Product show a safe tolerability profile.

In certain embodiments, the gene-editing of the NeoTCR Cells comprises electroporation of cells with a ribonucleoprotein (RNP) complex. In certain embodiments, the RNP complex comprises a guide RNA. In certain embodiments, the RNP complex comprises a nuclease. In certain embodiments, the nuclease is a Cas9 nuclease. In certain embodiments, the RNP complex targets endogenous loci. In certain embodiments, the RNP complex targets a TCR locus. In certain embodiments, the RNP complex targets a TRAC locus. In certain embodiments, the RNP complex targets a TRBC locus. In certain embodiments, the RNP complex target TRAC and TRBC loci.

In certain embodiments, the present disclosure methods for determining the safety profile of the NeoTCR Products disclosed herein. In certain non-limiting embodiments, for example, the safety profile can be assessed by detecting genomic instability (e.g., by performing targeted locus amplification (TLA) or standard FISH cytogenetics), or by detecting off-target integration (e.g., by Guide-Seq analysis).

The genome engineering approaches described herein enable highly efficient generation of bespoke NeoTCR T cells (i.e., NeoTCR Products) for personalized adoptive cell therapy for patients with solid and liquid tumors. Furthermore, the engineering methods are not restricted to the use in T cells and can be applied to other primary cell types, including natural killer and hematopoietic stem cells.

### 3. Therapeutic Composition and Methods of Manufacturing

Components

As described herein, plasmid DNA-mediated (non-viral) precision genome engineering processes for Good Manufacturing Practice (GMP) manufacturing of NeoTCR Product have been developed. For example, but not by way of limitation, targeted integration of a patient-specific NeoTCR can be accomplished by electroporating CRISPR endonuclease ribonucleoproteins (RNPs) together with a personalized NeoTCR gene cassette, encoded by a plasmid DNA.

In certain embodiments, a NeoTCR Product is formulated into a drug product using the clinical manufacturing processes described herein. Under these processes, the NeoTCR Product is cryopreserved in CryoMACS Freezing Bags. One or more bags can be shipped to the site for each patient depending on patient need. In certain embodiments, the product is composed of apheresis-derived, patient-autologous, CD8 and CD4 T cells that have been precision genome engineered to express one or more autologous NeoTCRs targeting a neoepitope complexed to one of the endogenous HLA receptors, presented, e.g., predominantly or exclusively, on the surface of that patient's tumor cells.

In certain embodiments, the final NeoTCR Product contains 5% dimethyl sulfoxide (DMSO), human serum albumin and Plasma-Lyte. In certain embodiments, the final NeoTCR Product contains the list of components provided in Table 1.

TABLE 1
Composition of the NeoTCR Product

| Component | Specification/Grade |
|---|---|
| Total nucleated NeoTCR cells | cGMP manufactured |
| Plasma-Lyte A | USP |
| Human Serum Albumin in 0.02-0.08M | USP |
| sodium caprylate and sodium tryptophanate | |
| CryoStor CS10 | cGMP manufactured with USP grade |
| | materials |

Product Packaging

In certain embodiments, the NeoTCR Product final drug product is provided to each clinical site in one or more CryoMACS 250 Freezing bags filled with the NeoTCR Product within the recommended fill volume of 30-70 mL.

The CryoMACS freezing bags are made of ethylene vinyl acetate tubular film. The bags are filled through the integrated tubing set containing an injection port and male and female luer lock assemblies which provide flexibility to use a variety of disposable transfer sets, syringes or Sterile Connecting Device for aseptic processing. As part of the bag assembly, two spike ports are available which allow access to the bag contents for therapeutic use of the product, via attachment of a sterile transfusion assembly. These spike ports are secured with sealed twist-off protective caps to prevent cross contamination and contain internal protection tubes which inhibit perforation of the freezing bag during removal of the thawed cell product.

In certain embodiments, the product label is attached to the bag, applied directly to the surface of the bag with a duplicate label attached to the storage cassette, prior to freezing to ensure traceability of the product to the specific patient. In addition, in certain embodiments, a truncated label containing essential information (i.e., a patient prescribed ID) is inserted into the label pocket of the freezing bag to permit identification in case primary label is missing or illegible for any reason.

In certain embodiments, a controlled rate freezing process was developed for the NeoTCR Product. A key element of this process is to not use the overwrap which is provided with the CryoMACS Freezing Bag. Accordingly, exemplary methods of cryopreserving the NeoTCR Product comprises filling a CryoMACS Freezing Bag with the NeoTCR Product and cryopreserving the NeoTCR Product using a controlled rate freezing profile without the use of the CryoMACS Freezing Bag overwrap. This does not affect how the CryoMACS bag is used in the clinic. In certain embodiments, a metal storage cassette protects the freezing bag during cryopreservation and transport.

Nonclinical Pharmacology

A list of the pharmacodynamic studies performed to evaluate the NeoTCR Product is presented in Table 2. Safety pharmacology studies are listed in Table 3.

TABLE 2
Pharmacology Studies for NeoTCR Product

| Type of | | Method of | |
|---|---|---|---|
| Study | Topic | Administration | Test System |
| Primary PD | Functional Characterization | Ex vivo assays with | Functional T cell |
| | of NeoTCR Product | human T cells from | assays: T cell |
| | | healthy donors | proliferation, killing, |
| | | | cytokine production |
| | | | and T cell phenotype |
| Primary PD | NeoTCR Products | Ex vivo assays with | Functional T cell |
| | generated from patients | human T cells from | assays: T cell |
| | with cancer and healthy | | proliferation, killing, |
| | donors: Comparison of T | healthy donors or | cytokine production |
| | cell phenotype and | patients with cancer | and T cell phenotype |
| | functionality | | |
| Primary PD | Comparison of phenotype | Ex vivo assays with | Functional T cell |
| | and functionality of cells | human T cells from | assays: T cell |
| | generated by small | healthy donors | proliferation, killing, |
| | (laboratory) scale or clinical | | cytokine production |
| | manufacturing process | | and T cell phenotype |

TABLE 3
Safety Pharmacology Studies for NeoTCR Product

| | | Method of | |
|---|---|---|---|
| Type of Study | Topic | Administration | Test System |
| Safety | Gene editing on-target | in vitro assays with human | TLA, FISH |
| Pharmacology | and off-target | T cells from healthy | |
| | integration and | donors | |
| | translocation analysis | | |
| Safety | Gene Editing | in vitro assays with human | Flow Cytometry |
| Pharmacology | Efficiencies; | T cells from healthy | |
| | Assessment of T cell | donors | |
| | Populations | | |
| Safety | NeoTCR Expression: | in vitro assays with human | TCR Expression |
| Pharmacology | Development and | T cells from healthy | Level by Flow |
| | Qualification | donors | Cytometry, |
| | | | Analysis of |
| | | | generated T cell |
| | | | species |
| Safety | Assessment of | in silico analysis, in vitro | COSMID, |
| Pharmacology | potential CRISPR off- | assay with human cell line, | GUIDE-seq, |
| | targets | and in vitro assays with | Targeted Deep |
| | | human T cells from | Sequencing |
| | | healthy donors | |
| Safety | Gene Editing Plasmid | in vitro assays with human | 2A Flow |
| Pharmacology | and Cloning | T cells from healthy | Cytometry |
| | | donors | Detection |
| Safety | Cas9 Impurity | in vitro assays with human | Cas9 ELISA |
| Pharmacology | Assessment | T cells from healthy | |
| | | donors | |

Pharmacodynamics

The pharmacodynamic studies described herein recapitulate antigen-specific T cell proliferation, cytokine production, and target T cell killing activity using ex vivo assays of precision genome engineered human NeoTCR-T cells (i.e, the NeoTCR Product).

Pharmacodynamic studies supporting the mechanism of action of the PACT NeoTCR Product have been performed with NeoTCR cells. The reagents include antibodies used for T cell selection, reagents for precision genome engineering, media, and cytokines. The starting material can be selected from either leukopaks or blood draws. Expansion of the T cells occurred in vessels such as a G-Rex vessel or a CentriCult vessel. Additional methods of cell expansion can take place in T flasks, culture bags, closed system bioreactors (non-limiting examples include the Xuri system (General Electric), the Ambr system (Sartorius), the Quantum system (Terumo CVT), and the Cocoon system (Lonza), cell stacks that are optionally optimized for non-adherent cells, and cell factories that are optionally optimized for non-adherent cells.

A direct comparison between the laboratory-scale and clinical-scale manufacturing and their effects on final product phenotype and functionality has been performed. The final product phenotype and functionality are comparable between the two manufacturing processes. One difference observed between laboratory-scale to clinical-scale production is that NeoTCR cells manufactured using the laboratory-scale process have a higher percentage of CD8 T cells compared to the cells produced using the clinical scale manufacturing process. This difference in CD8-to-CD4 T cell ratio is not anticipated to affect the functionality of the product in vivo, since the NeoTCR-T cells will proliferate in number upon encountering the cognate HLA-antigen. Based on these studies, data generated using NeoTCR-T cells produced at laboratory scale are concluded to be representative of the pharmacodynamic effects of NeoTCR Product manufactured from a patient leukopak for clinical use.

### 4. Therapeutic Methods

Dosing of the NeoTCR Products.

In certain embodiments, the total dose of cells administered to a patient is 4×10⁸ per infusion of NeoTCR Product. In certain embodiments, the total dose of cells administered to a patient is approximately about 4×10⁸ per infusion of NeoTCR Product.

In certain embodiments, the total dose of cells administered to a patient is 1.3×10⁹ per infusion of NeoTCR Product. In certain embodiments, the total dose of cells administered to a patient is approximately about 1.3×10⁹ per infusion of NeoTCR Product.

In certain embodiments, the total dose of cells administered to a patient is 4×10⁹ per infusion of NeoTCR Product. In certain embodiments, the total dose of cells administered to a patient is approximately about 4×10⁹ per infusion of NeoTCR Product.

In certain embodiments, the total dose of cells administered to a patient is between 4×10⁸ per infusion and 1.3×10⁹ per infusion of NeoTCR Product. In certain embodiments, the total dose of cells administered to a patient is between approximately about 4×10⁸ per infusion and approximately about 1.3×10⁹ per infusion of NeoTCR Product.

In certain embodiments, the total dose of cells administered to a patient is between 1.3×10⁹ per infusion and 4×10⁹ per infusion of NeoTCR Product. In certain embodiments, the total dose of cells administered to a patient is between approximately about 1.3×10⁹ per infusion and approximately about 4×10⁹ per infusion of NeoTCR Product.

In certain embodiments, the total dose of cells administered to a patient is greater than 4×10⁹ per infusion of NeoTCR Product.

In certain embodiments, the NeoTCR Product comprises one population of cells (i.e., the NeoTCR Product comprises NeoTCR cells that all express the same NeoTCR).

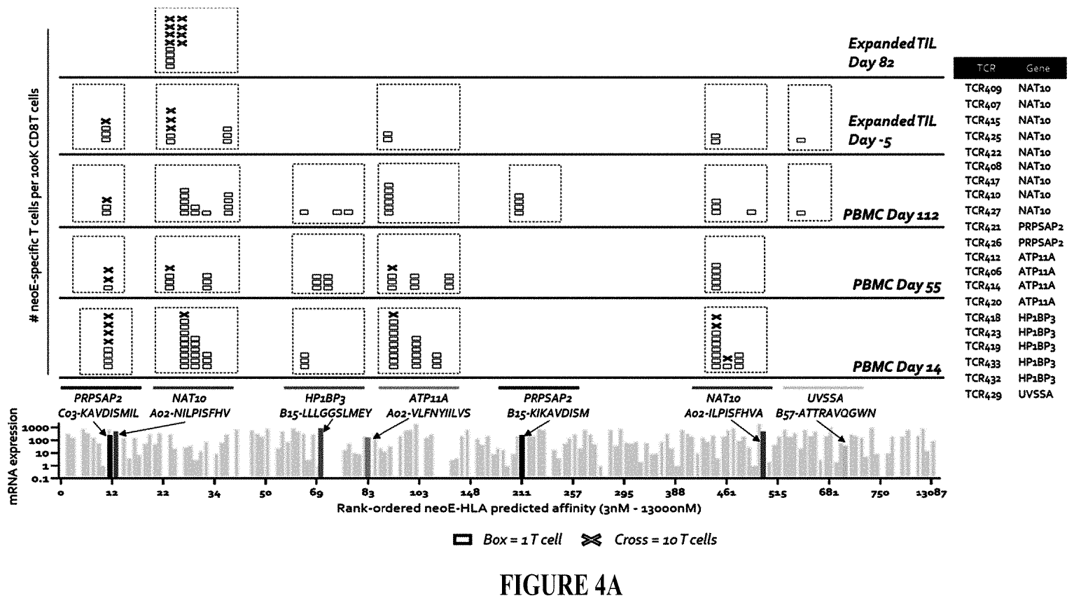

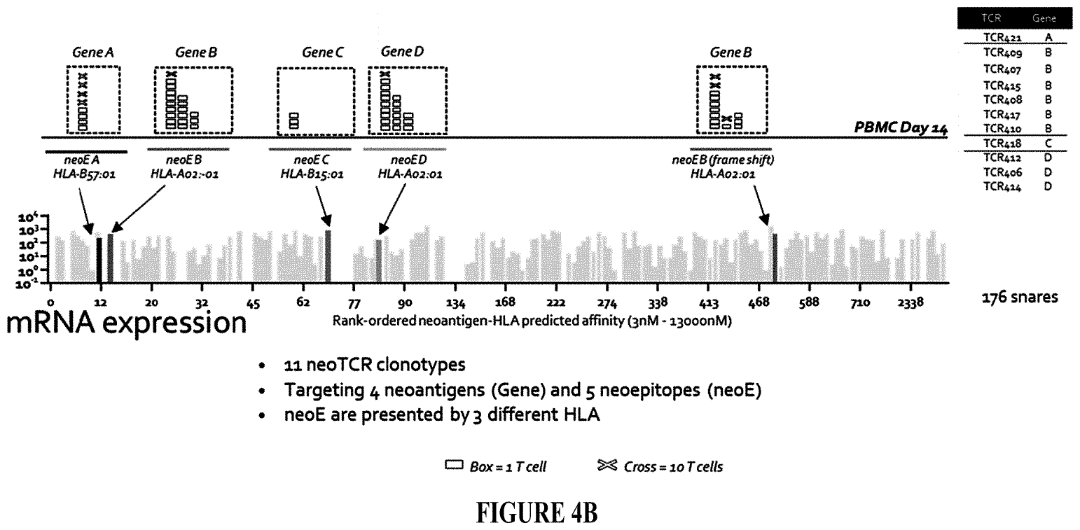

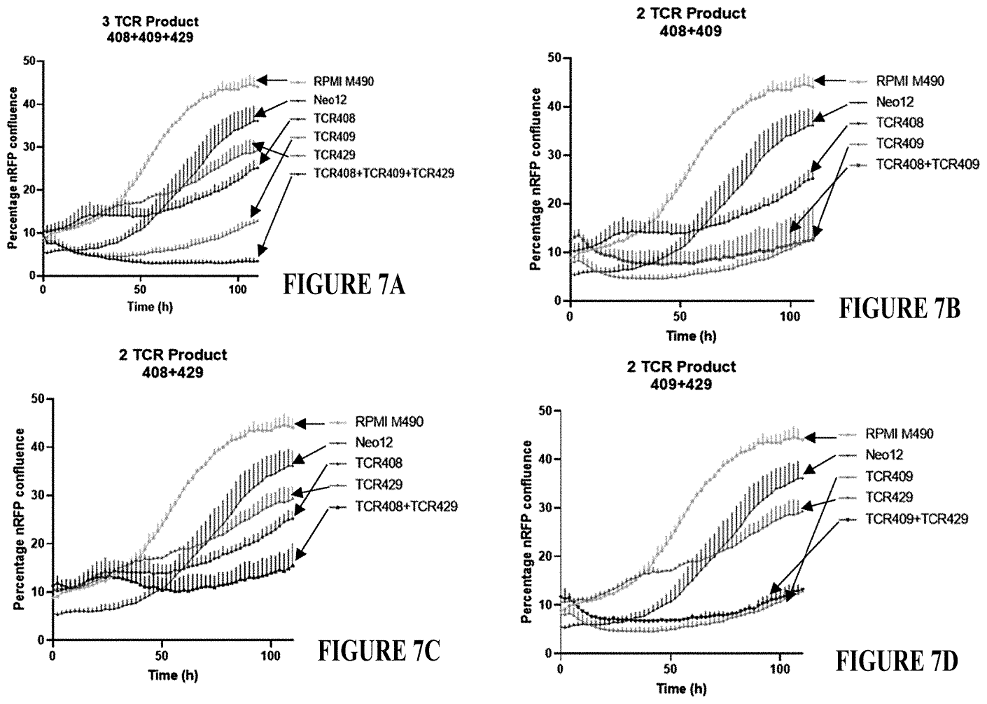

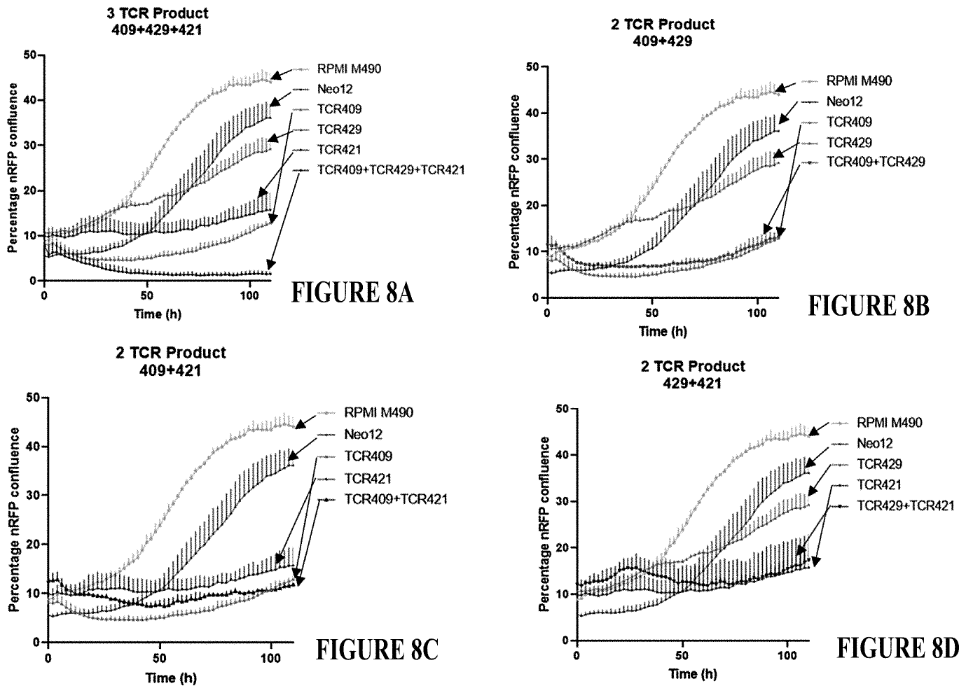

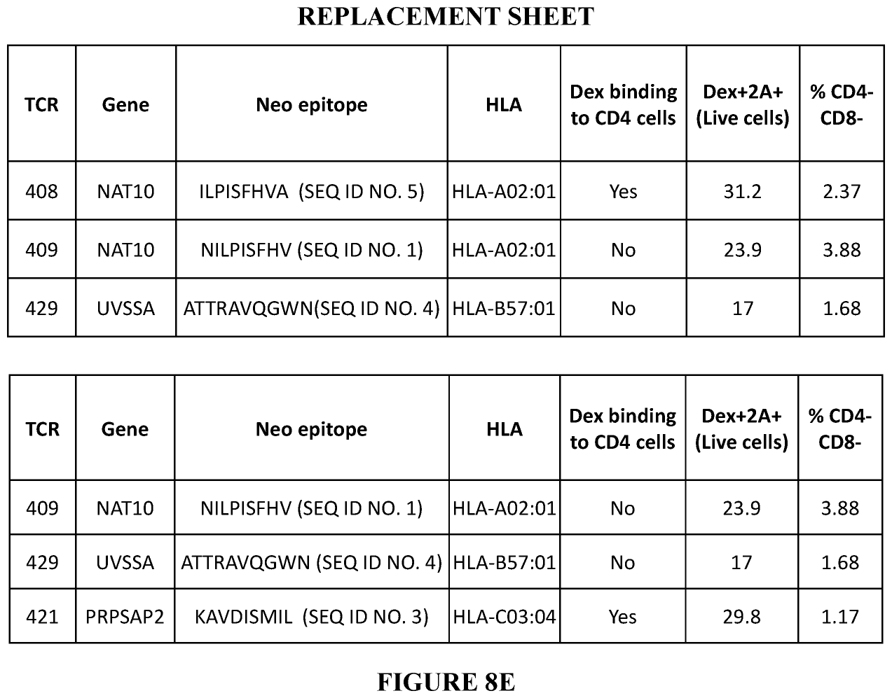

In certain embodiments, the NeoTCR Product comprises two populations of cells (i.e., the NeoTCR Product comprises two populations of NeoTCR cells: one population that expresses a first NeoTCR and a second population that expresses a second NeoTCR). In certain embodiments, each of the two populations of NeoTCRs express a NeoTCR specific to the same gene (e.g., see FIGS. 4A and 8E for examples of different NeoTCRs to the same gene and FIG. 7B for an example of a NeoTCR Product comprising 2 populations of NeoTCR Cells to the same gene). In certain embodiments, each of the two populations of NeoTCRs express a NeoTCR specific to two different genes (e.g., see FIGS. 7C, 7D, and 8E). In certain embodiments, each of the two populations of NeoTCRs express a NeoTCR specific to the same HLA (e.g., see FIGS. 4A and 8E for examples of different NeoTCRs to the same HLA and FIG. 7B for an example of a NeoTCR Product comprising 2 populations of NeoTCR Cells to the same HLA). In certain embodiments, each of the two populations of NeoTCRs express a NeoTCR specific to two different HLAs (e.g., see FIGS. 7C, 7D, and 8E).

In certain embodiments, the NeoTCR Product comprises three populations of cells (i.e., the NeoTCR Product comprises three populations of NeoTCR cells: one population that expresses a first NeoTCR, a second population that expresses a second NeoTCR, and a third population that expresses a third NeoTCR). In certain embodiments, each of the three populations of NeoTCRs express a NeoTCR specific to the same gene (e.g., see FIG. 4A for examples of different NeoTCRs to the same gene). In certain embodiments, each of the three populations of NeoTCRs express a NeoTCR specific to two different genes (e.g., see FIGS. 7A and 8E for examples of 2 NeoTCRs specific to one gene and 1 NeoTCR specific to a second gene). In certain embodiments, each of the three populations of NeoTCRs express a NeoTCR specific to three different genes (e.g., see FIGS. 8A and 8E for examples of 3 NeoTCRs each specific to a different gene). In certain embodiments, each of the three populations of NeoTCRs express a NeoTCR specific to the same HLA. In certain embodiments, each of the three populations of NeoTCRs express a NeoTCR specific to two different HLAs (e.g., see FIGS. 7A and 8E for examples of 2 NeoTCRs specific to one HLA and 1 NeoTCR specific to a second HLA). In certain embodiments, each of the three populations of NeoTCRs express a NeoTCR specific to three different HLAs (e.g., see FIGS. 8A and 8E for examples of 3 NeoTCRs each specific to a different HLA).

In certain embodiments, the NeoTCR Products comprise the approximate cell numbers described in Table 4 or Table 5.

TABLE 4
NeoTCR Products comprising 2 NeoTCR cell populations

| | Approximate | Approximate No. | Approximate No. |
|---|---|---|---|
| NeoTCR | Total No. of | of 1^{st} Population | of 2^{nd} Population |
| Product | Cells | of NeoTCR Cells | of NeoTCR Cells |
| 1 | 4 × 10⁸ | 2 × 10⁸ | 2 × 10⁸ |
| 2 | 4 × 10⁸ | Less than 2 × 10⁸ | 4 × 10⁸ - No. of 1^{st} |
| | | | Population of NeoTCR Cells |
| 3 | 4 × 10⁸ | 4 × 10⁸ - No. of 2^{nd} | Less than 2 × 10⁸ |
| | | Population of NeoTCR | |
| | | Cells | |
| 4 | 1.3 × 10⁹ | 6.5 × 10⁸ | 6.5 × 10⁸ |
| 5 | 1.3 × 10⁹ | Less than 6.5 × 10⁸ | 1.3 × 10⁹ - No. of 1^{st} |
| | | | Population of NeoTCR Cells |
| 6 | 1.3 × 10⁹ | 1.3 × 10⁹ - No. of 2^{nd} | Less than 6.5 × 10⁸ |
| | | Population of NeoTCR | |
| | | Cells | |
| 7 | 4 × 10⁹ | 2 × 10⁹ | 2 × 10⁹ |
| 8 | 4 × 10⁹ | Less than 2 × 10⁹ | 4 × 10⁹ - No. of 1^{st} |
| | | | Population of NeoTCR Cells |
| 9 | 4 × 10⁹ | 4 × 10⁹ - No. of 2^{nd} | Less than 2 × 10⁹ |
| | | Population of NeoTCR | |
| | | Cells | |
| 10 | Greater than | (Greater than 4 × 10⁹)/2 | (Greater than 4 × 10⁹)/2 |
| | 4 × 10⁹ | | |
| 11 | Greater than | Less than (Greater than | (Greater than 4 × 10⁹)/2 - |
| | 4 × 10⁹ | 4 × 10⁹)/2 | No. of 1^{st} Population of |
| | | | NeoTCR Cells |
| 12 | Greater than | (Greater than 4 × 10⁹)/2 - | Less than (Greater than |
| | 4 × 10⁹ | No. of 2^{nd} Population of | 4 × 10⁹)/2 |
| | | NeoTCR Cells | |

TABLE 5
NeoTCR Products comprising 3 NeoTCR cell populations

| | Approximate | Approximate No. | Approximate No. | Approximate No. |
|---|---|---|---|---|
| NeoTCR | Total No. of | of 1^{st} Population | of 2^{nd} Population | of 3^{rd} Population |
| Product | Cells | of NeoTCR Cells | of NeoTCR Cells | of NeoTCR Cells |
| 1 | 4 × 10⁸ | 1.33 × 10⁸ | 1.33 × 10⁸ | 1.33 × 10⁸ |
| 2 | 4 × 10⁸ | Less than 1.33 × 10⁸ | 1.33 × 10⁸ | 4 × 10⁸ - No. of 1^{st} |
| | | | | and 2^{nd} Population |
| | | | | of NeoTCR Cells |
| 3 | 4 × 10⁸ | Less than 1.33 × 10⁸ | Less than 1.33 × 10⁸ | 4 × 10⁸ - No. of 1^{st} |
| | | | | and 2^{nd} Population |
| | | | | of NeoTCR Cells |
| 4 | 4 × 10⁸ | 1.33 × 10⁸ | Less than 1.33 × 10⁸ | 4 × 10⁸ - No. of 1^{st} |
| | | | | and 2^{nd} Population |
| | | | | of NeoTCR Cells |
| 5 | 4 × 10⁸ | Less than 1.33 × 10⁸ | 4 × 10⁸ - No. of 1^{st} | 1.33 × 10⁸ |
| | | | and 3^{rd} Population | |
| | | | of NeoTCR Cells | |
| 6 | 4 × 10⁸ | 1.33 × 10⁸ | 4 × 10⁸ - No. of 1^{st} | Less than |
| | | | and 2^{nd} Population | 1.33 × 10⁸ |
| | | | of NeoTCR Cells | |
| 7 | 4 × 10⁸ | Less than 1.33 × 10⁸ | 4 × 10⁸ - No. of 1^{st} | Less than |
| | | | and 2^{nd} Population | 1.33 × 10⁸ |
| | | | of NeoTCR Cells | |
| 8 | 4 × 10⁸ | 4 × 10⁸ - No. of 2^{nd} | Less than 1.33 × 10⁸ | 1.33 × 10⁸ |
| | | and 3^{rd} Population | | |
| | | of NeoTCR Cells | | |
| 9 | 4 × 10⁸ | 4 × 10⁸ - No. of 2^{nd} | Less than 1.33 × 10⁸ | Less than |
| | | and 3^{rd} Population | | 1.33 × 10⁸ |
| | | of NeoTCR Cells | | |
| 10 | 4 × 10⁸ | 4 × 10⁸ - No. of 2^{nd} | 1.33 × 10⁸ | Less than |
| | | and 3^{rd} Population | | 1.33 × 10⁸ |
| | | of NeoTCR Cells | | |
| 11 | 1.3 × 10⁹ | Less than 4.3 × 10⁸ | 4.3 × 10⁸ | 1.3 × 10⁹ - No. of |
| | | | | 1^{st} and 2^{nd} |
| | | | | Population of |
| | | | | NeoTCR Cells |
| 12 | 1.3 × 10⁹ | Less than 4.3 × 10⁸ | Less than 4.3 × 10⁸ | 1.3 × 10⁹ - No. of |
| | | | | 1^{st} and 2^{nd} |
| | | | | Population of |
| | | | | NeoTCR Cells |
| 13 | 1.3 × 10⁹ | 4.3 × 10⁸ | Less than 4.3 × 10⁸ | 1.3 × 10⁹ - No. of |
| | | | | 1^{st} and 2^{nd} |
| | | | | Population of |
| | | | | NeoTCR Cells |
| 14 | 1.3 × 10⁹ | Less than 4.3 × 10⁸ | 1.3 × 10⁹ - No. of 1^{st} | 4.3 × 10⁸ |
| | | | and 3^{rd} Population | |
| | | | of NeoTCR Cells | |
| 15 | 1.3 × 10⁹ | 4.3 × 10⁸ | 1.3 × 10⁹ - No. of 1^{st} | Less than 4.3 × 10⁸ |
| | | | and 2^{nd} Population | |
| | | | of NeoTCR Cells | |
| 16 | 1.3 × 10⁹ | Less than 4.3 × 10⁸ | 1.3 × 10⁹ - No. of 1^{st} | Less than 4.3 × 10⁸ |
| | | | and 2^{nd} Population | |
| | | | of NeoTCR Cells | |
| 17 | 1.3 × 10⁹ | 1.3 × 10⁹ - No. of 2^{nd} | Less than 4.3 × 10⁸ | 4.3 × 10⁸ |
| | | and 3^{rd} Population | | |
| | | of NeoTCR Cells | | |
| 18 | 1.3 × 10⁹ | 1.3 × 10⁹ - No. of 2^{nd} | Less than 4.3 × 10⁸ | Less than 4.3 × 10⁸ |
| | | and 3^{rd} Population | | |
| | | of NeoTCR Cells | | |
| 19 | 1.3 × 10⁹ | 1.3 × 10⁹ - No. of 2^{nd} | 4.3 × 10⁸ | Less than 4.3 × 10⁸ |
| | | and 3^{rd} Population | | |
| | | of NeoTCR Cells | | |
| 20 | 4 × 10⁹ | Less than 1.33 × 10⁹ | 1.33 × 10⁹ | 4 × 10⁹ - No. of 1^{st} |
| | | | | and 2^{nd} Population |
| | | | | of NeoTCR Cells |
| 21 | 4 × 10⁹ | Less than 1.33 × 10⁹ | Less than 1.33 × 10⁹ | 4 × 10⁹ - No. of 1^{st} |
| | | | | and 2^{nd} Population |
| | | | | of NeoTCR Cells |
| 22 | 4 × 10⁹ | 1.33 × 10⁹ | Less than 1.33 × 10⁹ | 4 × 10⁹ - No. of 1^{st} |
| | | | | and 2^{nd} Population |
| | | | | of NeoTCR Cells |
| 23 | 4 × 10⁹ | Less than 1.33 × 10⁹ | 4 × 10⁹ - No. of 1^{st} | 1.33 × 10⁹ |
| | | | and 3^{rd} Population | |
| | | | of NeoTCR Cells | |
| 24 | 4 × 10⁸ | 1.33 × 10⁹ | 4 × 10⁹ - No. of 1^{st} | Less than |
| | | | and 2^{nd} Population | 1.33 × 10⁹ |
| | | | of NeoTCR Cells | |
| 25 | 4 × 10⁸ | Less than 1.33 × 10⁹ | 4 × 10⁹ - No. of 1^{st} | Less than |
| | | | and 2^{nd} Population | 1.33 × 10⁹ |
| | | | of NeoTCR Cells | |
| 26 | 4 × 10⁸ | 4 × 10⁹ - No. of 2^{nd} | Less than 1.33 × 10⁹ | 1.33 × 10⁹ |
| | | and 3^{rd} Population | | |
| | | of NeoTCR Cells | | |
| 27 | 4 × 10⁸ | 4 × 10⁹ - No. of 2^{nd} | Less than 1.33 × 10⁹ | Less than |
| | | and 3^{rd} Population | | 1.33 × 10⁹ |
| | | of NeoTCR Cells | | |
| 28 | 4 × 10⁹ | 4 × 10⁹ - No. of 2^{nd} | 1.33 × 10⁹ | Less than |
| | | and 3^{rd} Population | | 1.33 × 10⁹ |
| | | of NeoTCR Cells | | |
| 29 | Greater than | (Greater than | (Greater than | (Greater than |
| | 4 × 10⁹ | 4 × 10⁹)/3 | 4 × 10⁹)/3 | 4 × 10⁹)/3 |
| 30 | Greater than | Less than (Greater | (Greater than | (Greater than |
| | 4 × 10⁹ | than 4 × 10⁹)/2 | 4 × 10⁹)/3 | 4 × 10⁹)/3 - (No. |
| | | | | of 1^{st} Population |
| | | | | of NeoTCR Cells + |
| | | | | No. of 2^{nd} |
| | | | | Population of |
| | | | | NeoTCR Cells |
| 31 | Greater than | Less than (Greater | Less than (Greater | (Greater than |
| | 4 × 10⁹ | than 4 × 10⁹)/2 | than 4 × 10⁹)/2 | 4 × 10⁹)/3 - (No. |
| | | | | of 1^{st} Population |
| | | | | of NeoTCR Cells + |
| | | | | No. of 2^{nd} |
| | | | | Population of |
| | | | | NeoTCR Cells |
| 32 | Greater than | (Greater than | Less than (Greater | (Greater than |
| | 4 × 10⁹ | 4 × 10⁹)/3 | than 4 × 10⁹)/2 | 4 × 10⁹)/3 - (No. |
| | | | | of 1^{st} Population |
| | | | | of NeoTCR Cells + |
| | | | | No. of 2^{nd} |
| | | | | Population of |
| | | | | NeoTCR Cells |
| 33 | Greater than | (Greater than | (Greater than | Less than (Greater |
| | 4 × 10⁹ | 4 × 10⁹)/3 | 4 × 10⁹)/3 - (No. of | than 4 × 10⁹)/2 |
| | | | 1^{st} Population of | |
| | | | NeoTCR Cells + | |
| | | | No. of 3^{rd} | |
| | | | Population of | |
| | | | NeoTCR Cells | |
| 34 | Greater than | Less than (Greater | (Greater than | (Greater than |
| | 4 × 10⁹ | than 4 × 10⁹)/2 | 4 × 10⁹)/3 - (No. of | 4 × 10⁹)/3 |
| | | | 1^{st} Population of | |
| | | | NeoTCR Cells + | |
| | | | No. of 3^{rd} | |
| | | | Population of | |
| | | | NeoTCR Cells | |
| 35 | Greater than | Less than (Greater | (Greater than | Less than (Greater |
| | 4 × 10⁹ | than 4 × 10⁹)/2 | 4 × 10⁹)/3 - (No. of | than 4 × 10⁹)/2 |
| | | | 1^{st} Population of | |
| | | | NeoTCR Cells + | |
| | | | No. of 3^{rd} | |
| | | | Population of | |
| | | | NeoTCR Cells | |
| 36 | Greater than | (Greater than | (Greater than | Less than (Greater |
| | 4 × 10⁹ | 4 × 10⁹)/3 - (No. of | 4 × 10⁹)/3 | than 4 × 10⁹)/2 |
| | | 2^{nd} Population of | | |
| | | NeoTCR Cells + | | |
| | | No. of 3^{rd} | | |
| | | Population of | | |
| | | NeoTCR Cells | | |
| 37 | Greater than | (Greater than | Less than (Greater | (Greater than |
| | 4 × 10⁹ | 4 × 10⁹)/3 - (No. of | than 4 × 10⁹)/2 | 4 × 10⁹)/3 |
| | | 2^{nd} Population of | | |
| | | NeoTCR Cells + | | |
| | | No. of 3^{rd} | | |
| | | Population of | | |
| | | NeoTCR Cells | | |
| 38 | Greater than | (Greater than | Less than (Greater | Less than (Greater |
| | 4 × 10⁹ | 4 × 10⁹)/3 - (No. of | than 4 × 10⁹)/2 | than 4 × 10⁹)/2 |
| | | 2^{nd} Population of | | |
| | | NeoTCR Cells + | | |
| | | No. of 3^{rd} | | |
| | | Population of | | |
| | | NeoTCR Cells | | |

In both Tables 4 and 5, the “greater than 4×10⁹” is less than a total number of NeoTCR Cells that result in a dose limiting toxicity to a patient.

In certain embodiments, the NeoTCR Product comprises four populations of cells (i.e., the NeoTCR Product comprises three populations of NeoTCR cells: one population that expresses a first NeoTCR, a second population that expresses a second NeoTCR, a third population that expresses a third NeoTCR, and a fourth population that expresses a fourth NeoTCR). In certain embodiments, the NeoTCR Product that comprises four populations of cells comprises: 1) four populations of cells, each of which expresses a NeoTCR to the same gene, 2) four populations of cells, each of which expresses a NeoTCR to a different gene, 3) four populations of cells, one of which expresses a NeoTCR to a first gene and three of which express a NeoTCR to a second gene, or 4) four populations of cells, two of which expresses a NeoTCR to a first gene and two of which express a NeoTCR to a second gene. In certain embodiments, the NeoTCR Product that comprises four populations of cells comprises: 1) four populations of cells, each of which expresses a NeoTCR to the same HLA, 2) four populations of cells, each of which expresses a NeoTCR to a different HLA, 3) four populations of cells, one of which expresses a NeoTCR to a first HLA and three of which express a NeoTCR to a second HLA, or 4) four populations of cells, two of which expresses a NeoTCR to a first HLA and two of which express a NeoTCR to a second HLA. In certain embodiments, each of the four populations of NeoTCR Cells of the NeoTCR Product comprise approximately an equal number of cells. In certain embodiments, one of the four populations of NeoTCR Cells of the NeoTCR Product comprises less than ¼ of the total number of NeoTCR Cells in the NeoTCR Product and the other three populations of NeoTCR Cells are adjusted to provide a total number of NeoTCR cells in the NeoTCR Product to be approximately equal to 4×10⁸, 1.33×10⁹, 4×10⁹, >4×10⁸ and <1.33×10⁹, >1.33×10⁹ and <4×10⁹, or <4×10⁹ but less than a total number of NeoTCR Cells that result in a dose limiting toxicity to a patient. In certain embodiments, two of the four populations of NeoTCR Cells of the NeoTCR Product comprises less than ½ (less than ¼ each) of the total number of NeoTCR Cells in the NeoTCR Product and the other two populations of NeoTCR Cells are adjusted to provide a total number of NeoTCR cells in the NeoTCR Product to be approximately equal to 4×10⁸, 1.33×10⁹, 4×10⁹, >4×10⁸ and <1.33×10⁹, >1.33×10⁹ and <4×10⁹, or <4×10⁹ but less than a total number of NeoTCR Cells that result in a dose limiting toxicity to a patient. In certain embodiments, three of the four populations of NeoTCR Cells of the NeoTCR Product comprises less than ¾ (less than ¼ each) of the total number of NeoTCR Cells in the NeoTCR Product and the other one populations of NeoTCR Cells are adjusted to provide a total number of NeoTCR cells in the NeoTCR Product to be approximately equal to 4×10⁸, 1.33×10⁹, 4×10⁹, >4×10⁸ and <1.33×10⁹, >1.33×10⁹ and <4×10⁹, or <4×10⁹ but less than a total number of NeoTCR Cells that result in a dose limiting toxicity to a patient. In certain embodiments, one of the four populations of NeoTCR Cells of the NeoTCR Product comprises less than ¼ of the total number of NeoTCR Cells in the NeoTCR Product and the other three populations of NeoTCR Cells are adjusted to provide a total number of NeoTCR cells in the NeoTCR Product to be approximately equal to about 4×10⁸, about 1.33×10⁹, about 4×10⁹, greater about 4×10⁸ and less than about 1.33×10⁹, greater than about 1.33×10⁹ and less than about 4×10⁹, or less than about 4×10⁹ but less than a total number of NeoTCR Cells that result in a dose limiting toxicity to a patient. In certain embodiments, two of the four populations of NeoTCR Cells of the NeoTCR Product comprises less than ½ (less than ¼ each) of the total number of NeoTCR Cells in the NeoTCR Product and the other two populations of NeoTCR Cells are adjusted to provide a total number of NeoTCR cells in the NeoTCR Product to be approximately equal to about 4×10⁸, about 1.33×10⁹, about 4×10⁹, greater than about 4×10⁸ and less than about 1.33×10⁹, greater than about 1.33×10⁹ and less than about 4×10⁹, or less than about 4×10⁹ but less than a total number of NeoTCR Cells that result in a dose limiting toxicity to a patient. In certain embodiments, three of the four populations of NeoTCR Cells of the NeoTCR Product comprises less than ¾ (less than ¼ each) of the total number of NeoTCR Cells in the NeoTCR Product and the other one populations of NeoTCR Cells are adjusted to provide a total number of NeoTCR cells in the NeoTCR Product to be approximately equal to about 4×10⁸, about 1.33×10⁹, about 4×10⁹, greater than about 4×10⁸ and less than about 1.33×10⁹, greater than about 1.33×10⁹ and less than about 4×10⁹, or greater than about 4×10⁹ but less than a total number of NeoTCR Cells that result in a dose limiting toxicity to a patient.

In certain embodiments, a person of kill in the art could continue increasing the number of different NeoTCR Cell populations in the NeoTCR Product while keeping the total dose of NeoTCR Cells approximately equivalent to 4×10⁸, 1.33×10⁹, 4×10⁹, >4×10⁸ and <1.33×10⁹, >1.33×10⁹ and <4×10⁹, or <4×10⁹ but less than a total number of NeoTCR Cells that result in a dose limiting toxicity to a patient. In certain embodiments, a person of kill in the art could continue increasing the number of different NeoTCR Cell populations in the NeoTCR Product while keeping the total dose of NeoTCR Cells approximately equivalent to about 4×10⁸, about 1.33×10⁹, about 4×10⁹, greater than about 4×10⁸ and less than about 1.33×10⁹, greater than about 1.33×10⁹ and less than about 4×10⁹, or less than about 4×10⁹ but less than a total number of NeoTCR Cells that result in a dose limiting toxicity to a patient. However, the person of skill in the art shall also consider the minimum number of each NeoTCR Cell population needed to yield a clinical effect. Accordingly, the number of NeoTCR Cell populations per NeoTCR Product shall be limited by the need to provide a minimum number of each population of NeoTCR Cells.

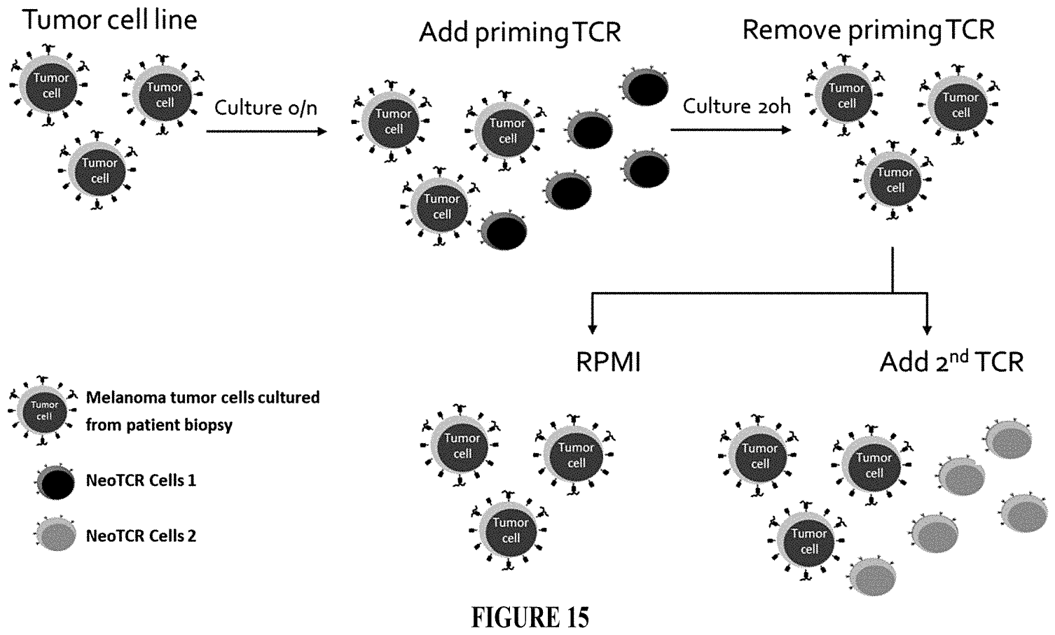

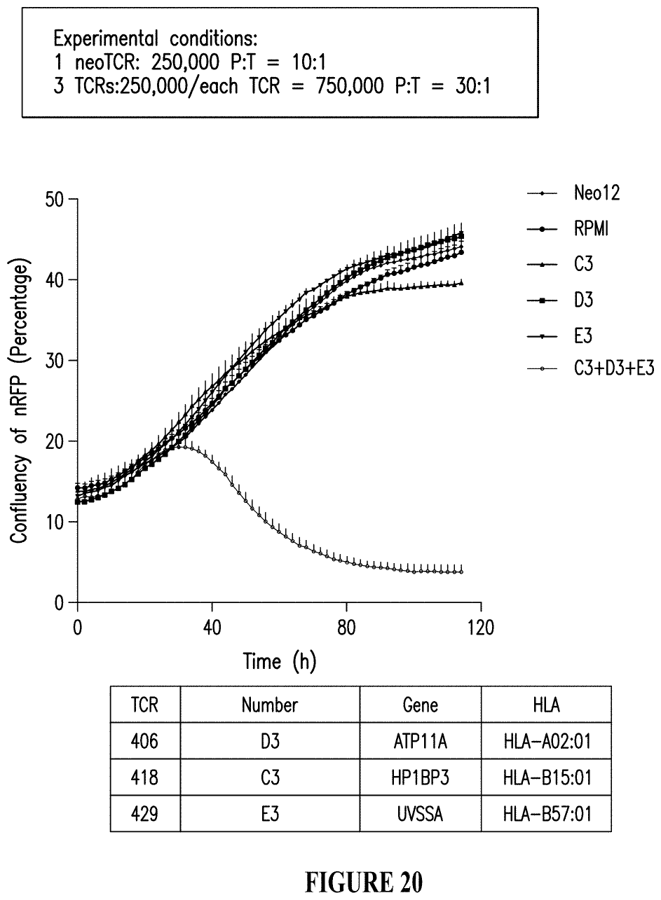

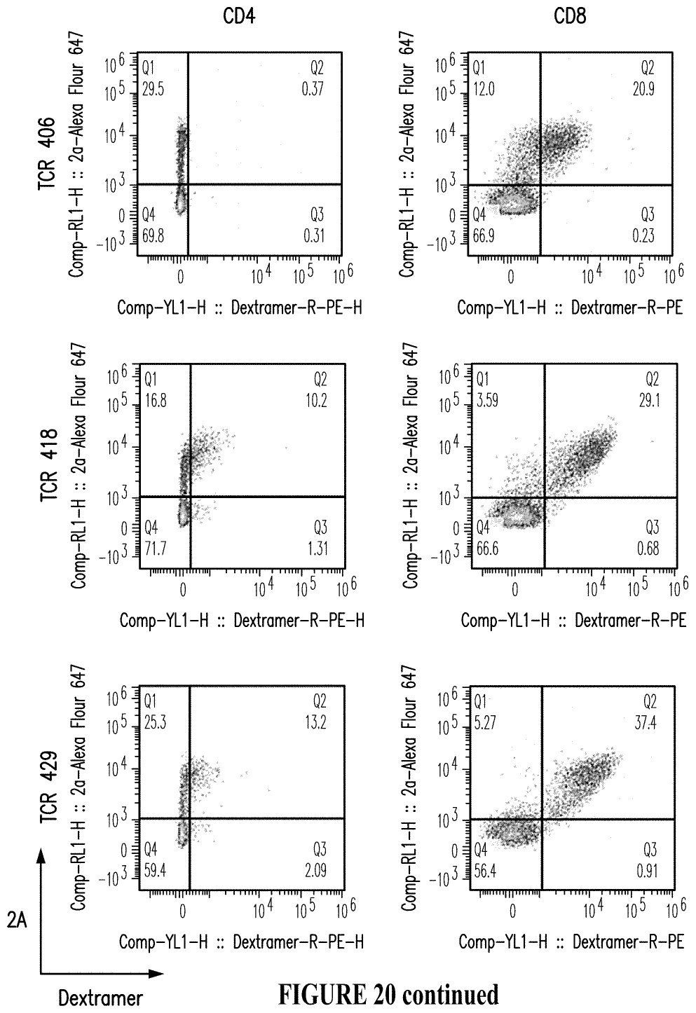

In certain embodiments, the three different NeoTCRs expressed in a NeoTCR Product comprising three different NeoTCR Cell populations result in a synergistic effect between the three NeoTCRs. In certain embodiments, this synergistic effect is a synergistic tumor cell killing effect compared to a one NeoTCR or two NeoTCR population NeoTCR Product. See, e.g., FIGS. 15, 18A-18D, and 20.

In certain embodiments, a NeoTCR Product is administered following a conditioning chemotherapy. In certain embodiments, the conditioning NeoTCR Product is a combination of Fludarabine and Cycophosphamide (Flu-Cy). In certain embodiments, the Flu-Cy is administered on days −5 through −3 before the NeoTCR Product infusion.

In certain embodiments the Flu-Cy is administered on days −5 and −4 before the NeoTCR Product infusion. In certain embodiments the Flu-Cy is administered on days −4 and −3 before the NeoTCR Product infusion. In certain embodiments the Flu-Cy is administered for three consecutive days one or more days prior to the administration of the NeoTCR Product. In certain embodiments the Flu-Cy is administered for three consecutive days two or more days prior to the administration of the NeoTCR Product. In certain embodiments the Flu-Cy is administered for three consecutive days three or more days prior to the administration of the NeoTCR Product. In certain embodiments the Flu-Cy is administered for three consecutive days three days prior to the administration of the NeoTCR Product. In certain embodiments the Flu-Cy is administered for two consecutive days one or more days prior to the administration of the NeoTCR Product. In certain embodiments the Flu-Cy is administered for two consecutive days two or more days prior to the administration of the NeoTCR Product. In certain embodiments the Flu-Cy is administered for two consecutive days three or more days prior to the administration of the NeoTCR Product. In certain embodiments the Flu-Cy is administered for two consecutive days three days prior to the administration of the NeoTCR Product.

In certain embodiments, the Flu-Cy is administered once a day for three consecutive days two days before the day the NeoTCR Product is administered.

In certain embodiments, for each day the Flu-Cy is administered to the patient, the fludarabine is administered intravenously at 30 mg/m² of body surface area and the cytophosphamide is administered at 300 mg/m² of body surface area. In certain embodiments, the fludarabine is reduced by 10% total amount infused. In certain embodiments, the fludarabine is reduced by 20% total amount infused. In certain embodiments, the fludarabine is reduced by 30% total amount infused. In certain embodiments, the fludarabine is reduced by 40% total amount infused. In certain embodiments, the fludarabine is reduced by 50% total amount infused. In certain embodiments, the fludarabine is reduced by 60% total amount infused. In certain embodiments, the fludarabine is reduced by about 10% total amount infused. In certain embodiments, the fludarabine is reduced by about 20% total amount infused. In certain embodiments, the fludarabine is reduced by about 30% total amount infused. In certain embodiments, the fludarabine is reduced by about 40% total amount infused. In certain embodiments, the fludarabine is reduced by about 50% total amount infused. In certain embodiments, the fludarabine is reduced by about 60% total amount infused. In certain embodiments, the cytophosphamide is administered at 300 mg/m² of body surface area regardless of the reduction of fludarabine.

In certain embodiments, for each day the Flu-Cy is administered to the patient, the Fludarabine is administered intravenously at 27 mg/m² of body surface area and the cytophosphamide is administered at 300 mg/m² of body surface area. In certain embodiments, for each day the Flu-Cy is administered to the patient, the Fludarabine is administered intravenously at 24 mg/m² of body surface area and the cytophosphamide is administered at 300 mg/m² of body surface area. In certain embodiments, for each day the Flu-Cy is administered to the patient, the Fludarabine is administered intravenously at 21 mg/m² of body surface area and the cytophosphamide is administered at 300 mg/m² of body surface area. In certain embodiments, for each day the Flu-Cy is administered to the patient, the Fludarabine is administered intravenously at 18 mg/m² of body surface area and the cytophosphamide is administered at 300 mg/m² of body surface area. In certain embodiments, for each day the Flu-Cy is administered to the patient, the Fludarabine is administered intravenously at 15 mg/m² of body surface area and the cytophosphamide is administered at 300 mg/m² of body surface area. In certain embodiments, for each day the Flu-Cy is administered to the patient, the Fludarabine is administered intravenously at 12 mg/m² of body surface area and the cytophosphamide is administered at 300 mg/m² or at about 300 mg/m² of body surface area.

In certain embodiments, for each day the Flu-Cy is administered to the patient, the Fludarabine is administered intravenously at about 27 mg/m² of body surface area and the cytophosphamide is administered at 300 mg/m² or at about 300 mg/m² of body surface area. In certain embodiments, for each day the Flu-Cy is administered to the patient, the Fludarabine is administered intravenously at about 24 mg/m² of body surface area and the cytophosphamide is administered at 300 mg/m² or at about 300 mg/m² of body surface area. In certain embodiments, for each day the Flu-Cy is administered to the patient, the Fludarabine is administered intravenously at about 21 mg/m² of body surface area and the cytophosphamide is administered at 300 mg/m² or at about 300 mg/m² of body surface area. In certain embodiments, for each day the Flu-Cy is administered to the patient, the Fludarabine is administered intravenously at about 18 mg/m² of body surface area and the cytophosphamide is administered at 300 mg/m² or at about 300 mg/m² of body surface area. In certain embodiments, for each day the Flu-Cy is administered to the patient, the Fludarabine is administered intravenously at about 15 mg/m² of body surface area and the cytophosphamide is administered at 300 mg/m² or at about 300 mg/m² of body surface area. In certain embodiments, for each day the Flu-Cy is administered to the patient, the Fludarabine is administered intravenously at about 12 mg/m² of body surface area and the cytophosphamide is administered at 300 mg/m² or at about 300 mg/m² of body surface area.

In certain embodiments, for each day the Flu-Cy is administered to the patient, the Fludarabine is administered daily, intravenously at 30 mg/m² or at about 30 mg/m² of body surface area for four consecutive days prior to the administration of the NeoTCR Product and the cytophosphamide is administered daily, intravenously at 600 mg/m² or at about 600 mg/m² of body surface area for three consecutive days prior to the administration of the NeoTCR Product. In certain embodiments, for each day the Flu-Cy is administered to the patient, the Fludarabine is administered daily, intravenously at 30 mg/m² or at about 30 mg/m² of body surface area for four consecutive days prior to the administration of the NeoTCR Product and the cytophosphamide is administered daily, intravenously at a dose between about 300 mg/m² to about 600 mg/m² of body surface area for three consecutive days prior to the administration of the NeoTCR Product.

In certain embodiments, for each day the Flu-Cy is administered to the patient, the Fludarabine is administered daily, intravenously at 25 mg/m² or at about 25 mg/m² of body surface area for four consecutive days prior to the administration of the NeoTCR Product and the cytophosphamide is administered daily, intravenously at 60 mg/kg or at about 60 mg/kg for two consecutive days prior to the administration of the NeoTCR Product. In certain embodiments, for each day the Flu-Cy is administered to the patient, the Fludarabine is administered daily, intravenously at 25 mg/m² or at about 25 mg/m² of body surface area for four consecutive days prior to the administration of the NeoTCR Product and the cytophosphamide is administered daily, intravenously at 60 mg/kg or at about 60 mg/kg for three consecutive days prior to the administration of the NeoTCR Product.

In certain embodiments, for each day the Flu-Cy is administered to the patient, the Fludarabine is administered daily, intravenously at 25 mg/m² or at about 25 mg/m² of body surface area for five consecutive days prior to the administration of the NeoTCR Product and the cytophosphamide is administered daily, intravenously at 60 mg/kg or at about 60 mg/kg for two consecutive days prior to the administration of the NeoTCR Product. In certain embodiments, for each day the Flu-Cy is administered to the patient, the Fludarabine is administered daily, intravenously at 25 mg/m² or at about 25 mg/m² of body surface area for five consecutive days prior to the administration of the NeoTCR Product and the cytophosphamide is administered daily, intravenously at 60 mg/kg or at about 60 mg/kg for three consecutive days prior to the administration of the NeoTCR Product.

In certain embodiments, for each day the Flu-Cy is administered to the patient, the Fludarabine is administered daily, intravenously at 25 mg/m² or at about 25 mg/m² of body surface area for six consecutive days prior to the administration of the NeoTCR Product and the cytophosphamide is administered daily, intravenously at 60 mg/kg or at about 60 mg/kg for two consecutive days prior to the administration of the NeoTCR Product. In certain embodiments, for each day the Flu-Cy is administered to the patient, the Fludarabine is administered daily, intravenously at 25 mg/m² or at about 25 mg/m² of body surface area for six consecutive days prior to the administration of the NeoTCR Product and the cytophosphamide is administered daily, intravenously at 60 mg/kg or at about 60 mg/kg for three consecutive days prior to the administration of the NeoTCR Product.

In certain embodiments, a subcutaneous IL-2 agent is administered concurrently with the administration of a NeoTCR Product.

In certain embodiments, a subcutaneous IL-2 agent is administered after the administration of a NeoTCR Product.

In certain embodiments, a subcutaneous IL-2 agent is administered after the administration of a NeoTCR Product and is administered at a dose of 500,000 IU/m² subcutaneously twice daily (BID) for seven days. In certain embodiments, a subcutaneous IL-2 agent is administered after the administration of a NeoTCR Product and is administered at a dose of about 500,000 IU/m² subcutaneously twice daily (BID) for 7 days. In certain embodiments, a subcutaneous IL-2 agent is administered after the administration of a NeoTCR Product and is administered at a dose of 500,000 IU/m² or about 500,000 IU/m² subcutaneously twice daily (BID) for 5 days. In certain embodiments, a subcutaneous IL-2 agent is administered after the administration of a NeoTCR Product and is administered at a dose of 500,000 IU/m² or about 500,000 IU/m² subcutaneously twice daily (BID) for 6 days. In certain embodiments, a subcutaneous IL-2 agent is administered after the administration of a NeoTCR Product and is administered at a dose of 500,000 IU/m² or about 500,000 IU/m² subcutaneously twice daily (BID) for 8 or 9 days. In certain embodiments, a subcutaneous IL-2 agent is administered after the administration of a NeoTCR Product and is administered at a dose of 500,000 IU/m² or about 500,000 IU/m² subcutaneously twice daily (BID) for 10 or 11 days. In certain embodiments, a subcutaneous IL-2 agent is administered after the administration of a NeoTCR Product and is administered at a dose of 500,000 IU/m² or about 500,000 IU/m² subcutaneously twice daily (BID) for 12 or 3 days. In certain embodiments, a subcutaneous IL-2 agent is administered after the administration of a NeoTCR Product and is administered at a dose of 500,000 IU/m² or about 500,000 IU/m² subcutaneously twice daily (BID) for 14 days. In certain embodiments, a subcutaneous IL-2 agent is administered after the administration of a NeoTCR Product and is administered at a dose of 500,000 IU/m² or about 500,000 IU/m² subcutaneously twice daily (BID) for any period between 15 and 20 days. In certain embodiments, a subcutaneous IL-2 agent is administered after the administration of a NeoTCR Product and is administered at a dose of 500,000 IU/m² or about 500,000 IU/m² subcutaneously twice daily (BID) for 20 days.

In certain embodiments, an IL-2 agent is administered after the administration of a NeoTCR Product and is administered at a dose of 600,000 IU/kg or about 600, IU/kg intravenously once daily for seven days. In certain embodiments, an IL-2 agent is administered after the administration of a NeoTCR Product and is administered at a dose of 600,000 IU/kg or about 600,000, IU/kg intravenously every 8 hours to tolerance. In certain embodiments, an IL-2 agent is administered after the administration of a NeoTCR Product and is administered at a dose of 720,000 IU/kg or about 720,000, IU/kg intravenously every 8 hours to tolerance for up to 14 consecutive doses over the course of 5 days post infusion of the NeoTCR Product. In certain embodiments, an IL-2 agent is administered after the administration of a NeoTCR Product and is administered at a dose of 720,000 IU/kg or about 720,000, IU/kg intravenously every 8 hours to tolerance. In certain embodiments, an IL-2 agent is administered after the administration of a NeoTCR Product and is administered at a dose of 720,000 IU/kg or about 720,000, IU/kg intravenously every 8 hours to tolerance for up to 14 consecutive doses over the course of 5 days post infusion of the NeoTCR Product. In certain embodiments, an IL-2 agent is administered after the administration of a NeoTCR Product and is administered at a dose of about 600,000 IU/kg or about 720,000 IU/kg intravenously every 8 hours to tolerance for up to 14 consecutive doses over the course of 5 days (a Cycle) post infusion of the NeoTCR Product followed by an optional second Cycle of the IL-2 agent after an approximate 8-10 day rest period following the completion of the first Cycle.

In certain embodiments, the IL-2 agent is aldesleukin or the biological equivalent thereof.

In certain embodiments, the IL-2 agent is a pegylated IL-2 agent.

Benefits of the NeoTCR Product and Limitations of Other Therapies.

There is increasing evidence that suggests that checkpoint therapy-responsive solid tumors are more likely to harbor a higher somatic mutational burden (resulting in expression of tumor-exclusive neoantigens), and the tumors exhibit higher CD8 T cell infiltration and/or exhibit pre-existing high PD-L1 tumor expression (Schumacher & Schreiber, 2015). Each of these features represents a higher potential for endogenous immunogenicity of these tumors, namely that the immune system in those patients will have likely initiated a significant T cell immune response prior to initiation of immunotherapy (Lawrence, et al, 2013); (Tumeh, et al, 2014); (Wargo, et al, 2017). The application of next generation deep sequencing of tumors and immunologic analysis of the endogenous tumor-targeted T cell response provided compelling evidence for the connection between cancer immunotherapy benefit, tumor mutational burden, and a pre-existing population of neoantigen-specific T cells. The neoantigen-specific population of T cells that specifically recognize and kill the tumor cells harboring these tumor-exclusive mutations (neoantigens) are proposed to be the main mediators of effective cancer immunotherapies to trigger clinical benefit (Tran, et al, 2017) (Schumacher & Schreiber, 2015).

Several approaches have recently been developed to significantly augment the magnitude of tumor-targeted T cells in patients with cancer. Treatment with chimeric antigen receptor (CAR)-engineered autologous T cells has yielded remarkable complete response rates of clinical benefit in patients with chemotherapy-refractory lymphomas and leukemias. The administration of autologous T cells expanded from tumor-infiltrating T cells (TILs) has been shown to induce clinical responses in patients with melanoma and with tumor types that are not traditionally responsive to checkpoint inhibitor drug therapy (e.g., cholangiocarcinoma, microsatellite stable [MSS] colorectal cancer [CRC], and hormone receptor positive [HR+] breast cancer (Paulson et al, 2018); (Tran, et al., 2016) (Tran, et al., 2014). Furthermore, the administration of engineered, autologous TCR-T cells directed against shared tumor or viral antigens that are expressed (namely the same antigens expressed in many patients, in contrast to neoantigens or neoepitopes which are private to the tumor in each individual patient) are showing evidence of durable responses in patients.

Personalized cancer mutation-targeted therapies, such as RNA-based or peptide-based vaccines targeting cancer neoantigens are a promising new therapeutic modality with potential to increase the magnitude of pre-existing mutation-targeted T cell responses as well as generate de novo immune responses against tumor neoantigens (Sahin & Türeci, 2018) (Ott, et al., 2017). Neoantigen cancer vaccines can be particularly beneficial in low mutational burden tumor types, such as prostate, MSS CRC, or other translocation-driven tumors, where insufficient numbers of T cell clones are primed intrinsically to tumor antigens. Nonetheless, the potential benefit of neoantigen cancer vaccines can be limited by an inability to authenticate predicted antigen targets prior to vaccine administration, by the variable response to T cell vaccines in the human population and the challenge of eradicating large or fast-growing tumors with a gradually (and hence too slowly) expanding anti-tumor T cell immune response.

Adoptive TCR-T cell therapy targeting neoepitopes holds the potential to overcome the limitations described above. The NeoTCR Product described herein is a novel adoptive TCR-T cell therapy engineered with autologous NeoTCRs of native sequence, identified and isolated from the patient's personal intrinsic T cell cancer immune response. Tumor-specific genomic alterations that initially represent founder (truncal) mutations in each patient, including ‘driver’ mutations for cancer pathology, expand in number and diversify over time as ‘branch’ or ‘passenger’ mutations in later stage malignancies. The spectrum of these accumulated tumor-specific mutations represents a unique private signature of targets for immune recognition in each cancer patient (private neoantigens). T cells that target these private and tumor-exclusive neoantigens (neoepitope or neoE-specific T cells) harbor the potential to exclusively target and kill the tumor cells, while ignoring healthy cells that do not express these tumor-specific mutations. In this way, the immune system of each patient engages the tumors and an appropriately scaled intrinsic immune response, when properly leveraged, has been shown to eradicate the tumors.

Since all cancers are driven by underlying founder or truncal mutations, adoptive NeoTCR T cell therapy targeting truncal neoepitopes holds the potential for treatment of any patient with cancer. In certain embodiments, the NeoTCR Product adoptive personalized cell therapy involves engineering an individual's own CD8 and CD4 T cells to express naturally occurring NeoTCRs that already recognize tumor-exclusive neoantigens. In certain embodiments, these NeoTCRs, therefore, are of native sequence, derived from pre-existing mutation-targeted CD8 T cells and are captured from peripheral blood by a proprietary isolation technology, which authenticates the tumor-exclusive neoE targets in each patient. In the manufacturing process, freshly derived CD4 and CD8 T cells from a leukopak of the same patient are precision genome engineered to express one NeoTCR in a manner that reconstitutes ‘native’ autologous T cell function and that has been validated to interact with the autologous patient predicted antigens throughout the selection process. The clinical benefit to participants with cancer thus stems from delivering a single dose of ex vivo engineered, tumor mutation-targeted autologous NeoTCR cells, thus providing the potential to trigger rapid and durable responses in patients, some of which have no curative treatment options.

Patients that harbor significant pre-existing T cell immunity can be more likely to benefit upon administration of any one of a PD-1 axis binding agent, a PD-1 binding agent, a PD-L1 binding agent, or a PD-L2 binding agent. Because NeoTCR Product administration represents a significant and comprehensive tumor-targeted T cell immune response for each study participant with cancer, the inclusion of a PD-1 axis binding agent, a PD-1 binding agent, a PD-L1 binding agent, or a PD-L2 binding agent is can provide meaningful clinical benefit in those patients whose tumors had already harbored PD-L1/PD-1/PD-L2 immune resistance prior to NeoTCR Product dosing, or who rapidly acquire PD-L1/PD-1/PD-L2 immune resistance following NeoTCR Product administration. Therefore, the present disclosure provides combination therapy of a PD-1 axis binding agent, a PD-1 binding agent, a PD-L1 binding agent, or a PD-L2 binding agent plus NeoTCR Product administration. In certain embodiments, the combination therapy comprises an anti-PD-1 antibody and the NeoTCR Product disclosed herein. In certain embodiments, the anti-PD-1 antibody is MDX-1106 (nivolumab). In certain embodiments, the anti-PD-1 antibody is is MK-3475 (pembrolizumab). In certain embodiments, the anti-PD-1 antibody is cemiplimab. In certain embodiments, the anti-PD-1 antibody is JTX-4014. In certain embodiments, the anti-PD-1 antibody is spartalizumab. In certain embodiments, the anti-PD-1 antibody is sintilimab. In certain embodiments, the anti-PD-1 antibody is tislelizumab. In certain embodiments, the anti-PD-1 antibody is toripalimab. In certain embodiments, the anti-PD-1 antibody is dostarlimab. In certain embodiments, the anti-PD-1 antibody is MGA012. In certain embodiments, the anti-PD-1 antibody is AMP-514. In certain embodiments, the anti-PD-1 antibody is CT-011 (pidilizumab). In certain embodiments, the anti-PD-1 antibody is AMP-224. In certain embodiments, the anti-PD-1 antibody is MED1-0680. In certain embodiments, the anti-PD-1 antibody is PDR001. In certain embodiments, the anti-PD-1 antibody is REGN2810. In certain embodiments, the anti-PD-1 antibody is BGB-108.

This combination therapy is predicted to overcome immune resistance as in the best circumstances of clinical benefit from immune checkpoint therapy when a significant pre-existing anti-tumor T cell immune response and immune resistance by higher tumor PD-1 axis expression levels has been documented in patients.

In certain non-limiting embodiments, the present disclosure provides combination therapy of an IL-2 agent and the NeoTCR Product disclosed herein. In certain embodiments, the IL-2 agent is a recombinant IL-2. In certain embodiments, the IL-2 agent is a modified IL-2. In certain embodiments, the IL-2 agent is a PEGylated IL-2. In certain embodiments, the IL-2 agent is an antibody fused to IL-2. In certain embodiments, the IL-2 agent is a long-acting IL-2. In certain embodiments, the IL-2 agent is a short-acting IL-2. In certain embodiments, the IL-2 agent is aldesleukin. In certain embodiments, the IL-2 agent is Proleukin®. In certain embodiments, the IL-2 agent is bempegaldesleukin.

In certain non-limiting embodiments, the present disclosure provides combination therapy of an IL-15 agent and the NeoTCR Product disclosed herein. In certain embodiments, the IL-15 agent is a recombinant IL-15. In certain embodiments, the IL-15 agent is a modified IL-15. In certain embodiments, the IL-15 agent is a PEGylated IL-15. In certain embodiments, the IL-15 agent is an antibody fused to IL-15. In certain embodiments, the IL-15 agent is a long-acting IL-15. In certain embodiments, the IL-15 agent is a short-acting IL-15. In certain embodiments, the IL-15 agent is ALT-803. In certain embodiments, the IL-15 agent is NKTR-255. In certain embodiments, the IL-15 agent is NL-201. In certain embodiments, the IL-15 agent is SOC101.

In certain embodiments, the present disclosure comprises a chemotherapy preconditioning regimen administered prior to the administration of the NeoTCR Product.

In certain embodiments, the NeoTCR Product described herein comprises NeoTCR cells with potent antigen-specific killing, effector cytokine secretion, and proliferative activity upon contact with cognate neoantigen-expressing tumor cells. Furthermore, in certain embodiments, the NeoTCR Product responds to target tumor cells with a strong polyfunctional effector protein secretion response (e.g., as demonstrated by bulk T cell and single-cell secretome analysis). The polyfunctional T cell effector phenotype is predicted to contribute to the potential for clinical benefit upon infusion of NeoTCR Product into patients with cancer in a manner similar to that observed with polyfunctional CAR-T cells infused into patients with hematologic malignancies.

In certain embodiments, the NeoTCR Product comprises memory stem cell (T_{MSC}) and central memory (T_{CM}) T cell phenotypes as a result of the ex vivo manufacturing process described herein. These ‘younger’ or less-differentiated T cell phenotypes are described to confer improved engraftment potential and prolonged persistence post-infusion in mouse models and in clinical trials of engineered CAR-T cells in patients with hematologic malignancies. Thus, the administration of NeoTCR Product, comprising ‘younger’ T cell phenotypes, has the potential to benefit patients with cancer, through improved engraftment potential, prolonged persistence post-infusion, and rapid differentiation into effector T cells to eradicate tumor cells throughout the body.

In certain embodiments, the present disclosure comprises the NeoTCR Product manufacturing process which involves electroporation of dual ribonucleoprotein species of CRISPR-Cas9 nucleases bound to guide RNA sequences, with each species targeting the genomic TCRα and the genomic TCRβ loci. The specificity of targeting Cas9 nucleases to each genomic locus has been previously described in the literature as being highly specific. In certain embodiments, the present disclosure comprises comprehensive testing of the NeoTCR Product to survey possible off-target genomic cleavage sites. For example, without any limitation, the off-target site can be detected by using COSMID and GUIDE-seq.

Further aspects of the precision genome engineering process have been assessed for safety. In certain embodiments, the compositions disclosed herein show no evidence of genomic instability following precision genome engineering. In certain non-limiting embodiments, for example, genomic instability can be assessed by targeted locus amplification (TLA) or standard FISH cytogenetics.

The comprehensive assessment of the NeoTCR Product and precision genome engineering process indicates that the NeoTCR Product are well tolerated following infusion back to the patient.

### 5. Pharmaceutical Formulations

Pharmaceutical formulations of the NeoTCR Product are prepared by combining the NeoTCR cells in a solution that can preserve the ‘young’ phenotype of the cells in a cryopreserved state. Table 1 above provides an example of one such pharmaceutical formulation. Alternatively, pharmaceutical formulations of the NeoTCR Product can be prepared by combining the NeoTCR cells in a solution that can preserve the ‘young’ phenotype of the cells without the need to freeze or cryopreserve the product (i.e., the NeoTCR Product is maintained in an aqueous solution or as a non-frozen/cryopreserved cell pellet).

Additional pharmaceutically acceptable carriers, buffers, stabilizers, and/or preservatives can also be added to the cryopreservation solution or the aqueous storage solution (if the NeoTCR Product is not cryopreserved). Any cryopreservation agent and/or media can be used to cryopreserve the NeoTCR Product, including but not limited to CryoStor, CryoStor CSS, CELLBANKER, and custom cryopreservation medias that optionally include DMSO.

In certain embodiments, the pharmaceutical formulations of the present disclosure comprise a plurality of cell populations comprising a first and second population of modified cells. In certain embodiments, the first modified cell comprises a first NeoTCR which binds to a first antigen. In certain embodiments, the second modified cell comprises a second NeoTCR which binds to a second antigen. In certain embodiments, the plurality of cells exhibits cytotoxic activity toward cells comprising the first and second antigens. In certain embodiments, the plurality of cells comprises a third modified cell. In certain embodiments, the third modified cell comprises a third NeoTCR which binds to a third antigen. In certain embodiments, the plurality of cells exhibits cytotoxic activity towards cells comprising the first, second, and third antigens. In certain non-limiting embodiments, the plurality of cells comprises four or more modified cells, five or more modified cells, ten or more modified cells.

In certain embodiments, the NeoTCR Products of the present disclosure comprise a first and second population of NeoTCR Cells. In certain embodiments, the first NeoTCR Cell targets a first antigen. In certain embodiments, the second NeoTCR Cell targets a second antigen. In certain embodiments, the NeoTCR Cells exhibit cytotoxic activity toward cells comprising the first and second antigens. In certain embodiments, the NeoTCR Products comprise a third NeoTCR Cell. In certain embodiments, the third NeoTCR Cell targets a third antigen. In certain embodiments, the NeoTCR Product comprising the first, second and third NeoTCR Cells exhibits cytotoxic activity towards cells comprising the first, second, and third antigens. In certain non-limiting embodiments, the NeoTCR Products comprise four or more NeoTCR Cells, five or more NeoTCR Cells, ten or more NeoTCR Cells.

### 6. Articles of Manufacture

The NeoTCR Product can be used in combination of articles of manufacture. Such articles of manufacture can be useful for the prevention or treatment of proliferative disorders (e.g., cancer). Examples of articles of manufacture include but are not limited to containers (e.g., infusion bags, bottles, storage containers, flasks, vials, syringes, tubes, and IV solution bags) and a label or package insert on or associated with the container. In certain embodiments, the containers are made of any material that is acceptable for the storage and preservation of the NeoTCR cells in the ‘young’ state within the NeoTCR Product. In certain embodiments, the container is an intravenous solution bag or a vial having a stopper pierceable by a hypodermic injection needle. In certain embodiments, the container is a CryoMACS freezing bag. In certain embodiments, the label or package insert indicates that the NeoTCR Product is used for treating the condition of choice and the patient of origin. In certain embodiments, the patient is identified on the container of the NeoTCR Product because the NeoTCR Product is made from autologous cells and engineered as a patient-specific, individualized treatment.

In certain embodiments, the article of manufacture comprises: 1) a first container with a NeoTCR Product contained therein.

In certain embodiments, the article of manufacture comprises: 1) a first container with a NeoTCR Product contained therein; and 2) a second container with the same NeoTCR Product as the first container contained therein. Optionally, in certain embodiments, additional containers with the same NeoTCR Product as the first and second container are prepared and made. Optionally, in certain embodiments, additional containers containing a composition comprising a different cytotoxic or otherwise therapeutic agent are combined with the containers described above.

In certain embodiments, the article of manufacture comprises: 1) a first container with a NeoTCR Product contained therein; and 2) a second container with a composition contained therein, wherein the composition comprises a further cytotoxic or otherwise therapeutic agent.

In certain embodiments, the article of manufacture comprises: 1) a first container with two NeoTCR Products contained therein (i.e., a NeoTCR Product that comprises two populations of NeoTCR Cells wherein each population of NeoTCR Cells express a different NeoTCR (e.g., see Table 4); and 2) a second container with a composition contained therein, wherein the composition comprises a further cytotoxic or otherwise therapeutic agent. For clarity, for products comprising two populations of NeoTCR Cells, each population can each be referred to as a NeoTCR Product or the two populations of NeoTCR Cells in combination (whether combined into a single container or packaged in separate containers) can be referred to as a NeoTCR Product.

In certain embodiments, the article of manufacture comprises: 1) a first container with a NeoTCR Product contained therein comprising a first population of NeoTCR Cells; 2) a second container with a second NeoTCR Product contained therein comprising a second population of NeoTCR Cells; and 3) optionally a third container with a composition contained therein, wherein the composition comprises a further cytotoxic or otherwise therapeutic agent. In certain embodiments, the first and second NeoTCR Products are different NeoTCR Products wherein the first NeoTCR Product comprises NeoTCR Cells that express a first NeoTCR and the second NeoTCR Product comprises NeoTCR Cells that express a different NeoTCR than the first population of NeoTCR cells. In certain embodiments, the first and second NeoTCR Products are the same NeoTCR Products.

In certain embodiments, the article of manufacture comprises: 1) a first container with three NeoTCR Products contained therein (i.e., a NeoTCR Product that comprises three populations of NeoTCR Cells wherein each population of NeoTCR Cells express a different NeoTCR (e.g., see Table 5); and 2) optionally a second container with a composition contained therein, wherein the composition comprises a further cytotoxic or otherwise therapeutic agent.

In certain embodiments, the article of manufacture comprises: 1) a first container with a NeoTCR Product contained therein comprising a first population of NeoTCR Cells; 2) a second container with a second NeoTCR Product contained therein comprising a second population of NeoTCR Cells; 3) a third container with a third NeoTCR Product contained therein comprising a third population of NeoTCR Cells; and 4) optionally a fourth container with a composition contained therein, wherein the composition comprises a further cytotoxic or otherwise therapeutic agent. In certain embodiments, the first, second, and third NeoTCR Products are different NeoTCR Products wherein the first NeoTCR Product comprises NeoTCR Cells that express a first NeoTCR, the second NeoTCR Product comprises NeoTCR Cells that express a different NeoTCR than the first population of NeoTCR cells, and the third NeoTCR Product comprises NeoTCR Cells that express a different NeoTCR than the first and second population of NeoTCR cells. In certain embodiments, the first, second, and third NeoTCR Products are the same NeoTCR Products. In certain embodiments, two of the first, second, and third NeoTCR Products are the same NeoTCR Products. For clarity, for products comprising three populations of NeoTCR Cells, each population can be referred to as a NeoTCR Product or the three populations of NeoTCR Cells in combination (whether combined into a single container or packaged in separate containers) can be referred to as a NeoTCR Product.

In certain embodiments, the article of manufacture comprises: 1) a first container with four NeoTCR Products contained therein (i.e., four NeoTCR Cell populations); and 2) optionally a second container with a composition contained therein, wherein the composition comprises a further cytotoxic or otherwise therapeutic agent.

In certain embodiments, the article of manufacture comprises: 1) a first container with a NeoTCR Product contained therein; 2) a second container with a second NeoTCR Product contained therein; 3) a third container with a third NeoTCR Product contained therein; 4) a fourth container with a fourth NeoTCR Product contained therein; and 5) optionally a fifth container with a composition contained therein, wherein the composition comprises a further cytotoxic or otherwise therapeutic agent. In certain embodiments, the first, second, third, and fourth NeoTCR Products are different NeoTCR Products. In certain embodiments, the first, second, third, and fourth NeoTCR Products are the same NeoTCR Products. In certain embodiments, two of the first, second, third, and fourth NeoTCR Products are the same NeoTCR Products. In certain embodiments, three of the first, second, third, and fourth NeoTCR Products are the same NeoTCR Products. For clarity, for products comprising four populations of NeoTCR Cells, each population can be referred to as a NeoTCR Product or the four populations of NeoTCR Cells in combination (whether combined into a single container or packaged in separate containers) can be referred to as a NeoTCR Product.

In certain embodiments, the article of manufacture comprises: 1) a first container with five or more NeoTCR Products contained therein (i.e., five NeoTCR Cell populations); and 2) optionally a second container with a composition contained therein, wherein the composition comprises a further cytotoxic or otherwise therapeutic agent.

In certain embodiments, the article of manufacture comprises: 1) a first container with a NeoTCR Product contained therein; 2) a second container with a second NeoTCR Product contained therein; 3) a third container with a third NeoTCR Product contained therein; 4) a fourth container with a fourth NeoTCR Product contained therein; 5) a fifth container with a fifth NeoTCR Product contained therein; 6) optionally a sixth or more additional containers with a sixth or more NeoTCR Product contained therein; and 7) optionally an additional container with a composition contained therein, wherein the composition comprises a further cytotoxic or otherwise therapeutic agent. In certain embodiments, the all of the containers of NeoTCR Products are different NeoTCR Products. In certain embodiments, all of the containers of NeoTCR Products are the same NeoTCR Products. In certain embodiments, there can be any combination of same or different NeoTCR Products in the five or more containers based on the availability of detectable NeoTCRs in a patient's tumor sample(s), the need and/or desire to have multiple NeoTCR Products for the patient, and the availability of any one NeoTCR Product that require or benefit from one or more container. For clarity, for products comprising five populations of NeoTCR Cells, each population can be referred to as a NeoTCR Product or the five populations of NeoTCR Cells in combination (whether combined into a single container or packaged in separate containers) can be referred to as a NeoTCR Product.

Furthermore, In certain embodiments, any container of NeoTCR Product described herein can be split into two, three, or four separate containers for multiple time points of administration and/or based on the appropriate dose for the patient.

In certain embodiments, the NeoTCR Products are provided in a kit. The kit can, by means of non-limiting examples, contain package insert(s), labels, instructions for using the NeoTCR Product(s), syringes, disposal instructions, administration instructions, tubing, needles, and anything else a clinician would need in order to properly administer the NeoTCR Product(s).

### 7. Genome Editing Methods

In certain embodiments, the present disclosure involves, in part, methods of engineering human cells, e.g., engineered T cells or engineered human stem cells. In certain embodiments, the present disclosure involves, in part, methods of engineering human cells, e.g., NK cells, NKT cells, macrophages, hematopoietic stem cells (HSCs), cells derived from HSCs, or dendritic/antigen-presenting cells. In certain embodiments, such engineering involves genome editing. For example, but not by way of limitation, such genome editing can be accomplished with nucleases targeting one or more endogenous loci, e.g., TCR alpha (TCRα) locus and TCR beta (TCRβ) locus. In certain embodiments, the nucleases can generate single-stranded DNA nicks or double-stranded DNA breaks in an endogenous target sequence. In certain embodiments, the nuclease can target coding or non-coding portions of the genome, e.g., exons, introns. In certain embodiments, the nucleases contemplated herein comprise homing endonuclease, meganuclease, megaTAL nuclease, transcription activator-like effector nuclease (TALEN), zinc-finger nuclease (ZFN), and clustered regularly interspaced short palindromic repeats (CRISPR)/Cas nuclease. In certain embodiments, the nucleases can themselves be engineered, e.g., via the introduction of amino acid substitutions and/or deletions, to increase the efficiency of the cutting activity.

In certain embodiments, the genome editing is performed by using non-viral delivery systems. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

In certain embodiments, genome editing is performed by using viral delivery systems. In certain embodiments, the viral methods include targeted integration (including but not limited to AAV) and random integration (including but not limited to lentiviral approaches). In certain embodiments, the viral delivery would be accomplished without integration of the nuclease. In such embodiments, the viral delivery system can be Lentiflash or another similar delivery system.

Additional information concerning the genome editing methods useful in the context of the present disclosure can be found in International Patent Application no. PCT/US2018/058230, e.g. at paragraphs [00219]-[00226], the content of which is herein incorporated by reference for all purposes.

### 8. Homology Recombination Templates

In certain embodiments, the present disclosure provides methods for genome editing of cells by introducing and recombining a homologous recombination (HR) template nucleic acid sequence into an endogenous locus of a cell. In certain embodiments, the HR template nucleic acid sequence is linear. In certain embodiments, the HR template nucleic acid sequence is circular. In certain embodiments, the circular HR template can be a plasmid, minicircle, or nanoplasmid. In certain embodiments, the HR template nucleic acid sequence comprises a first and a second homology arms. In certain embodiments, the homology arms can be of about 300 bases to about 2,000 bases. For example, each homology arm can be 1,000 bases. In certain embodiments, the homology arms can be homologous to a first and second endogenous sequences of the cell. In certain embodiments, the endogenous locus is a TCR locus. For example, the first and second endogenous sequences are within a TCR alpha locus or a TCR beta locus. In certain embodiments, the HR template comprises a TCR gene sequences. In non-limiting embodiments, the TCR gene sequence is a patient specific TCR gene sequence. In non-limiting embodiments, the TCR gene sequence is tumor-specific. In non-limiting embodiments, the TCR gene sequence can be identified and obtained using the methods described in PCT/US2020/017887, the content of which is herein incorporated by reference. In certain embodiments, the HR template comprises a TCR alpha gene sequence and a TCR beta gene sequence.

In certain embodiments, the HR template is a polycistronic polynucleotide. In certain embodiments, the HR template comprises sequences encoding for flexible polypeptide sequences (e.g., Gly-Ser-Gly sequence). In certain embodiments, the HR template comprises sequences encoding an internal ribosome entry site (IRES). In certain embodiments, the HR template comprises a sequence coding a 2A peptide (e.g., P2A, T2A, E2A, and F2A). In certain embodiments, the HR template comprises codon-diverged sequences coding for the same amino acid sequence. For example, without any limitation, the HR template comprises a first and a second codon-diverged sequences coding for a first and second 2A peptides having the same amino acid sequence, e.g., P2A. Additional information on the HR template nucleic acids and methods of modifying a cell thereof can be found in International Patent Application no. PCT/US2018/058230, the content of which is herein incorporated by reference.

### 9. Kits

The present disclosure provides kits for inducing and/or enhancing an immune response and/or treating and/or preventing a cancer in a subject in need thereof. In certain embodiments, the kit comprises an effective amount of presently disclosed plurality of cells or a pharmaceutical composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding a presently disclosed HR template.

In certain embodiments, the kit comprises the consumable supplies required for the infusion of the NeoTCR Product; including but not limited to 1) a filling assembly comprising luer lock connectors, roller clamps, PVC tubing, and an injection port and 2) CryoMACS bag(s) (or equivalent bag) filled with the NeoTCR Product(s) that are optionally further contained in an overwrap bag and that are labelled with the patient identifying information to ensure the NeoTCR Product(s) is infused into the patient for which it was personally designed and manufactured. In certain embodiments, the kit further comprises instructions for how to thaw the NeoTCR Product(s) in a warm water bath and how to evenly suspend the NeoTCR Cells in the thawed NeoTCR Product(s) in the CryoMACS bag(s) (or equivalent bag).

If desired, the plurality of cells is provided together with instructions including information about the use for the treatment and/or prevention of a cancer. In certain embodiments, the instructions include at least one of the following: description of the therapeutic agent; dosage schedule and administration for treatment or prevention of a neoplasia, pathogen infection, or immune disorder or symptoms thereof; precautions; warnings; indications; counter-indications; over-dosage information; adverse reactions; animal pharmacology; clinical studies; and/or references. The instructions can be printed directly on the container (when present), or as a label applied to the container, or as a separate sheet, pamphlet, card, or folder supplied in or with the container.

In certain embodiments, the NeoTCR Product is filled and stored in a CryoMACS bag(s) (or equivalent bag) with a total viable cell concentration of 10-100×10⁴ cells/mL. In certain embodiments, the NeoTCR Product is filled and stored in a CryoMACS bag(s) (or equivalent bag) with a total viable cell concentration of 10-100×10⁴ cells/mL with a total volume of 35 mL. In certain embodiments one bag is filled and stored. In certain embodiments, two bags are filled and stored.

In certain embodiments, the NeoTCR Product is shipped in a CryoMACS bag(s) (or equivalent bag) with a total viable cell concentration of 10-100×10⁴ cells/mL. In certain embodiments, the NeoTCR Product is shipped in a CryoMACS bag(s) (or equivalent bag) with a total viable cell concentration of 10-100×10⁴ cells/mL with a total volume of 35 mL. In certain embodiments one bag is filled and shipped. In certain embodiments, two bags are filled and shipped.

In certain embodiments, the label on the NeoTCR Product reads “Total Viable Cell Concentration 10-100×10⁴ cells/mL”. In certain embodiments, the label on the NeoTCR Product reads “Total Viable Cell Concentration 10-100×10⁴ cells/mL, 35 mL”.

In certain embodiments, the NeoTCR Product is provided in a kit that includes instructions on how to calculate the correct milliliters of the product to infuse into the patient in order to infuse the cell numbers and doses set forth in Tables 4 and 5.

In certain embodiments, the NeoTCR Product is provided in a kit that comprises instructions on how to thaw the cryopreserved product. In certain embodiments, the instructions on how to thaw the NeoTCR Product comprise one or more the following steps (with alternate wording with the same meaning acceptable):
1) Remove one bag of the NeoTCR Product from the liquid nitrogen freezer with the cassette and place on dry ice to transport to the water bath.
2) Remove the cryobag (e.g., a CryoMACS bag) containing the NeoTCR Product from the cassette.
3) Place the NeoTCR Product cryobag in a new resealable (e.g. Ziplock-style) plastic bag. Seal the resealable bag containing the NeoTCR Product.
4) Submerge the NeoTCR Product cryobag (that is sealed within the resealable bag) in a water bath at temperature of 37° C.±2° C. Do not move the bag after submerging it the water bath.
5) Observe the progress of the thawing of the NeoTCR Product cryobag and remove from water bath when a small piece of ice remains. Rock the bag gently until the NeoTCR Product is completely thawed.
6) Remove the NeoTCR Product cryobag from the resealable bag.
7) Gently dry the outside of the bag with an absorbent disposable wipe to remove any condensation. Observe the cryobag for any leaks and to ensure the integrity of the cryobag is visually verified. If the integrity has been compromised, do not proceed with infusion.
8) Repeat thawing procedure for additional cryobags of the NeoTCR Product, as applicable, to achieve the prescribed dose.

In certain embodiments, the NeoTCR Product is provided with instruction to use the product within 4 hours of thawing.

### 10. Exemplary Embodiments

In certain embodiments, the present disclosure provides a composition comprising: a) a first NeoTCR cell population comprising a first NeoTCR that binds a first neoantigen; b) a first NeoTCR cell population comprising a first NeoTCR that binds a first neoantigen, and a second NeoTCR cell population comprising a second NeoTCR that binds a second neoantigen; or c) a first NeoTCR cell population comprising a first NeoTCR that binds a first neoantigen, a second NeoTCR cell population comprising a second NeoTCR that binds a second neoantigen, and a third NeoTCR cell population comprising a third NeoTCR that binds a third neoantigen; wherein each NeoTCR is different from the others, and wherein each NeoTCR is derived from a patient.

In certain embodiments of the compositions described herein, the first, second, and/or third neoantigens are expressed by a single gene. In certain embodiments of the compositions described herein, the first, second, and/or third neoantigens are expressed by different genes. In certain embodiments of the compositions described herein, the first, second, and/or third neoantigens are expressed by a single gene. In certain embodiments of the compositions described herein, the first, second, and/or third NeoTCRs bind to a single major histocompatibility complex. In certain embodiments of the compositions described herein, the first, second, and/or third NeoTCRs bind to different major histocompatibility complexes. In certain embodiments of the compositions described herein, two of the first, second, and/or third NeoTCRs bind to a single major histocompatibility complex.

In certain embodiments of the compositions described herein, the composition comprises a pharmaceutically acceptable carrier. In certain embodiments of the compositions described herein, the composition comprises a cryopreservation agent. In certain embodiments of the compositions described herein, the composition comprises serum albumin. In certain embodiments of the compositions described herein, the composition comprises a crystalloid solution. In certain embodiments of the compositions described herein, the composition comprises Plasma-Lyte A, human serum album (HAS), and CryoStor® CS10.

In certain embodiments, the present disclosure provides a method of treating a cancer in a subject in need thereof, comprising administering any of the compositions described herein.

In certain embodiments of the methods described herein, the composition comprises an amount of NeoTCR Cells of about 4×10⁸ cells, 1.33×10⁹ cells, or about 4×10⁹ cells. In certain embodiments of the methods described herein, the composition comprises an amount of NeoTCR Cells greater than about 4×10⁸ cells and less than about 1.33×10⁹ cells, greater than about 1.33×10⁹ cells and less than about 4×10⁹ cells, or greater than about 4×10⁹ cells. In certain embodiments of the methods described herein, the composition comprises an amount of NeoTCR cells according to Table 4. In certain embodiments of the methods described herein, the composition comprises an amount of NeoTCR cells according to Table 5. In certain embodiments of the methods described herein, the composition is administered in a single dose. In certain embodiments of the methods described herein, the composition is administered in multiple doses.

In certain embodiments of the methods described herein, the method further comprises administering a combination agent. In certain embodiments of the methods described herein, the combination agent is a cytokine, a PD-axis binding agent, a PD-1 binding agent, a PD-L1 binding agent, a PD-L2 binding agent, or a combination thereof. In certain embodiments of the methods described herein, the cytokine is an IL-2 agent, an IL-7 agent, an IL-10 agent, an IL-12 agent, an IL-15 agent, an IL-18 agent, an IL-21 agent, or a combination thereof. In certain embodiments of the methods described herein, the cytokine is an IL-2 agent. In certain embodiments of the methods described herein, the cytokine is an IL-15 agent. In certain embodiments of the methods described herein, the PD-axis binding agent comprises nivolumab, pembrolizumab, or atezolizumab.

In certain embodiments of the methods described herein, the cancer is a liquid cancer or a solid cancer. In certain embodiments of the methods described herein, the patient is provided a Flu-Cy conditioning treatment described herein.

In certain embodiments, the present disclosure provides a method of manufacturing any of the compositions described herein. In certain embodiments, the present disclosure provides a kit for the administration of any of the compositions described herein.

In certain embodiments, the present disclosure provides a plurality of cells comprising: a) a first modified cell comprising a first exogenous polynucleotide encoding a first NeoTCR binding a first antigen, wherein the first exogenous polynucleotide is integrated in an endogenous TRAC and/or TRBC locus of the first modified cell; and b) a second modified cell comprising a second exogenous polynucleotide encoding a second NeoTCR binding a second antigen, wherein the second exogenous polynucleotide is integrated in an endogenous TRAC and/or TRBC locus of the second modified cell.

In certain embodiments of the plurality of cells described herein, the composition further comprises a third modified cell comprising a third exogenous polynucleotide encoding a third NeoTCR binding a third antigen, wherein the third exogenous polynucleotide is integrated in an endogenous TRAC and/or TRBC locus of the third modified cell. In certain embodiments of the plurality of cells described herein, the first, second, and third NeoTCRs are patient derived. In certain embodiments of the plurality of cells described herein, the first, second, and third antigens are cancer antigens. In certain embodiments of the plurality of cells described herein, the cancer antigens are neoantigens.

In certain embodiments of the plurality of cells described herein, the cancer antigens are patient-specific antigens. In certain embodiments of the plurality of cells described herein, the first, second, and third modified cells are primary cells. In certain embodiments of the plurality of cells described herein, the primary cells are patient-derived cells. In certain embodiments of the plurality of cells described herein, the primary cells are lymphocytes. In certain embodiments of the plurality of cells described herein, the primary cells are T cells. In certain embodiments of the plurality of cells described herein, the T cells are CD45RA+, CD62L+, CD28+, CD95-, CCR7+, and CD27+. In certain embodiments of the plurality of cells described herein, the T cells are CD45RA+, CD62L+, CD28+, CD95+, CD27+, CCR7+. In certain embodiments of the plurality of cells described herein, the T cells are CD45RO+, CD62L+, CD28+, CD95+, CCR7+, CD27+, CD127+.

In certain embodiments of the plurality of cells described herein, the first, second, and third exogenous polynucleotides comprise a signal sequence, a first and second 2A-coding sequence, and a TCR gene sequence. In certain embodiments of the plurality of cells described herein, the TCR gene sequence of the first, second, and third exogenous polynucleotides are positioned between the first and second 2A-coding sequence. In certain embodiments of the plurality of cells described herein, the first and second 2A-coding sequences code for the same amino acid sequence and are codon-diverged relative to each other. In certain embodiments of the plurality of cells described herein, the first and second 2A-coding sequences are P2A-coding sequences.

In certain embodiments of the plurality of cells described herein, the exogenous polynucleotides further comprise a sequence encoding for the amino acid sequence Gly Ser Gly positioned immediately upstream of the first and/or second 2A-coding sequence. In certain embodiments of the plurality of cells described herein, the exogenous polynucleotides further comprise a sequence encoding for a Furin cleavage site positioned upstream of the second 2A-coding sequence. In certain embodiments of the plurality of cells described herein, the first, second, and third exogenous polynucleotides further comprises a second TCR gene. In certain embodiments of the plurality of cells described herein, the second TCR gene of the first, second, and third exogenous polynucleotides is positioned downstream of the second 2A-coding sequence. In certain embodiments of the plurality of cells described herein, the first, second, and third exogenous polynucleotides are circular polynucleotides.

In certain embodiments, the present disclosure provides a composition comprising the plurality of cells described herein. In certain embodiments of the compositions described herein, the composition further comprises a pharmaceutically acceptable excipient. In certain embodiments of the compositions described herein, the composition is administered to a patient in need thereof for the treatment of cancer.

In certain embodiments of the compositions described herein, the composition comprises a cryopreservation agent. In certain embodiments of the compositions described herein, the composition comprises serum albumin. In certain embodiments of the compositions described herein, the composition comprises a crystalloid solution. In certain embodiments of the compositions described herein, the composition comprises Plasma-Lyte A, human serum album (HAS), and CryoStor® CS10.

In certain embodiments, the present disclosure provides a method of treating a cancer in a subject in need thereof, the method comprising administering a plurality of cells, comprising a first modified cell comprising a first exogenous polynucleotide encoding a first NeoTCR binding a first antigen, wherein the first exogenous polynucleotide is integrated in an endogenous TRAC and/or TRBC locus of the first modified cell; and a second modified cell comprising a second exogenous polynucleotide encoding a second NeoTCR binding a second antigen, wherein the second exogenous polynucleotide is integrated in an endogenous TRAC and/or TRBC locus of the second modified cell; thereby treating the cancer in the subject.

In certain embodiments of the methods described herein, the plurality of cells further comprises a third modified cell comprising a third exogenous polynucleotide encoding a third NeoTCR binding a third antigen, wherein the third exogenous polynucleotide is integrated in an endogenous TRAC and/or TRBC locus of the third modified cell.

In certain embodiments of the methods described herein, the first, second, and third NeoTCRs are patient derived. In certain embodiments of the methods described herein, the first, second, and third antigens are cancer antigens. In certain embodiments of the methods described herein, the cancer antigens are neoantigens. In certain embodiments of the methods described herein, the cancer antigens are patient-specific antigens.

In certain embodiments of the methods described herein, the first, second, and third modified cells are primary cells. In certain embodiments of the methods described herein, the primary cells are patient-derived cells. In certain embodiments of the methods described herein, the primary cells are lymphocytes. In certain embodiments of the methods described herein, the primary cells are T cells. In certain embodiments of the methods described herein, the T cells are CD45RA+, CD62L+, CD28+, CD95-, CCR7+, and CD27+. In certain embodiments of the methods described herein, the T cells are CD45RA+, CD62L+, CD28+, CD95+, CD27+, CCR7+. In certain embodiments of the methods described herein, the T cells are CD45RO+, CD62L+, CD28+, CD95+, CCR7+, CD27+, CD127+.

In certain embodiments of the methods described herein, the first, second, and third exogenous polynucleotides comprise a signal sequence, a first and second 2A-coding sequence, and a TCR gene sequence. In certain embodiments of the methods described herein, the TCR gene sequence of the first, second, and third exogenous polynucleotides are positioned between the first and second 2A-coding sequence. In certain embodiments of the methods described herein, the first and second 2A-coding sequences code for the same amino acid sequence and are codon-diverged relative to each other. In certain embodiments of the methods described herein, the first and second 2A-coding sequences are P2A-coding sequences.

In certain embodiments of the methods described herein, the exogenous polynucleotides further comprise a sequence encoding for the amino acid sequence Gly Ser Gly positioned immediately upstream of the first and/or second 2A-coding sequence. In certain embodiments of the methods described herein, the exogenous polynucleotides further comprise a sequence encoding for a Furin cleavage site positioned upstream of the second 2A-coding sequence.

In certain embodiments of the methods described herein, the first, second, and third exogenous polynucleotides further comprises a second TCR gene. In certain embodiments of the methods described herein, the second TCR gene of the first, second, and third exogenous polynucleotides is positioned downstream of the second 2A-coding sequence. In certain embodiments of the methods described herein, the first, second, and third exogenous polynucleotides are circular polynucleotides.

In certain embodiments of the methods described herein, the method further comprises administering a combination agent. In certain embodiments of the methods described herein, the combination agent is a cytokine, a PD-axis binding agent, a PD-1 binding agent, a PD-L1 binding agent, a PD-L2 binding agent, or a combination thereof. In certain embodiments of the methods described herein, the cytokine is an IL-2 agent, an IL-7 agent, an IL-10 agent, an IL-12 agent, an IL-15 agent, an IL-18 agent, an IL-21 agent, or a combination thereof. In certain embodiments of the methods described herein, the cytokine is an IL-2 agent. In certain embodiments of the methods described herein, the cytokine is an IL-15 agent. In certain embodiments of the methods described herein, the PD-axis binding agent is nivolumab, pembrolizumab, atezolizumab, or a combination thereof.

In certain embodiments of the methods described herein, the cancer is a liquid cancer. In certain embodiments of the methods described herein, the liquid cancer is selected from the group consisting of follicular lymphoma, leukemia, and multiple myeloma. In certain embodiments of the methods described herein, the cancer is a solid cancer. In certain embodiments of the methods described herein, the solid cancer is selected from the group consisting of melanoma, thoracic cancer, lung cancer, ovarian cancer, breast cancer, pancreatic cancer, head and neck cancer, prostate cancer, gynecological cancer, central nervous system cancer, cutaneous cancer, HPV+ cancer, esophageal cancer, thyroid cancer, gastric cancer, hepatocellular cancer, cholangiocarcinoma, renal cell cancer, bladder cancer, testicular cancer, sarcoma, and colorectal cancer.

In certain embodiments, the present disclosure provides a method of treating a cancer in a subject in need thereof, the method comprising administering an effective amount of a modified cell comprising an exogenous polynucleotide encoding a NeoTCR binding a tumor antigen, wherein the exogenous polynucleotide is integrated in an endogenous TRAC and/or TRBC locus of the modified cell; and administering an effective amount of a combination agent; thereby treating the cancer in the subject. In certain embodiments of the methods described herein, the combination agent comprises a chemotherapeutic agent, an anti-hormonal agent, an endocrine therapeutic, a cytotoxic agent, a cytokine, a PD-axis binding agent, a PD-1 binding agent, a PD-L1 binding agent, a PD-L2 binding agent, or a combination thereof.

In certain embodiments of the methods described herein, the cytokine is an IL-2 agent, an IL-7 agent, an IL-10 agent, an IL-12 agent, an IL-15 agent, an IL-18 agent, an IL-21 agent, or a combination thereof. In certain embodiments of the methods described herein, the cytokine is an IL-2 agent. In certain embodiments of the methods described herein, the cytokine is an IL-15 agent. In certain embodiments of the methods described herein, the PD-axis binding agent comprises nivolumab, pembrolizumab, or atezolizumab.

In certain embodiments of the methods described herein, the cancer is a liquid cancer. In certain embodiments of the methods described herein, the liquid cancer is selected from the group consisting of follicular lymphoma, leukemia, and multiple myeloma. In certain embodiments of the methods described herein, the cancer is a solid cancer. In certain embodiments of the methods described herein, the solid cancer is selected from the group consisting of melanoma, thoracic cancer, lung cancer, ovarian cancer, breast cancer, pancreatic cancer, head and neck cancer, prostate cancer, gynecological cancer, central nervous system cancer, cutaneous cancer, HPV+ cancer, esophageal cancer, thyroid cancer, gastric cancer, hepatocellular cancer, cholangiocarcinoma, renal cell cancer, bladder cancer, testicular cancer, sarcoma, and colorectal cancer.

### EXAMPLES

The following are examples of methods and compositions of the invention. It is understood that various other embodiments can be practiced, given the general description provided above.

### Example 1. The NeoTCR Product

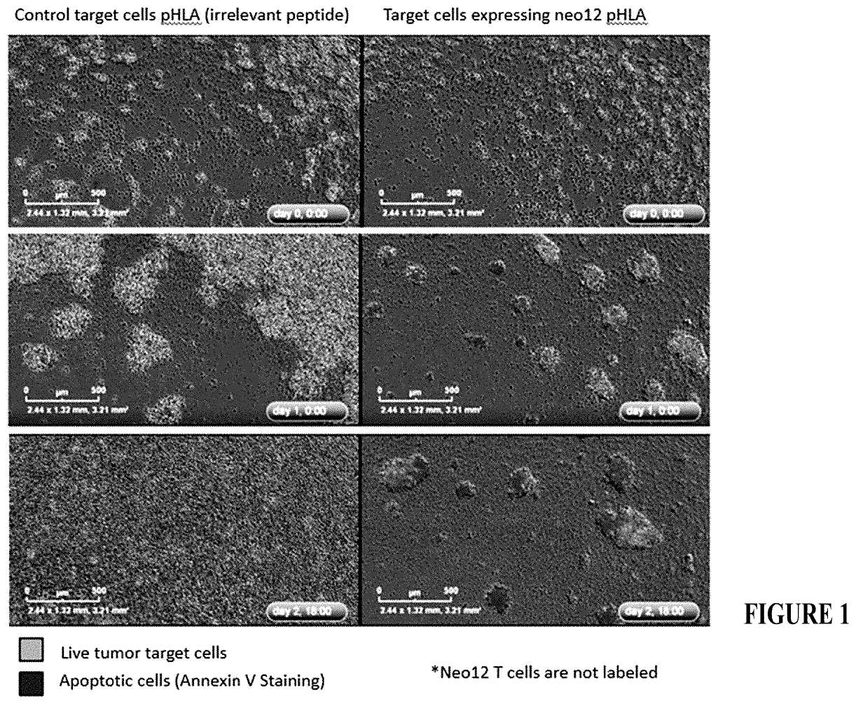

Antigen-specific target T cell killing. Mutation-targeted, personalized, adoptive, T cell receptor (NeoTCR Product) therapy is an immunotherapy modality designed to unleash the immune system's ability to specifically recognize and kill cells displaying tumor-exclusive mutational targets. NeoTCR cells were engineered to express the neo12 TCR (a representative neoepitope) generated from neo12-specific CD8 T cells isolated from the blood of a patient with melanoma and co-cultured with cognate or unmatched tumor cells for several days and time-lapse live microscopy images were collected. Representative images obtained with time-lapse live microscopy (day 0, 1, and 2; see FIG. 1) demonstrate potent antigen-specific cytotoxic activity and proliferation by NeoTCR-T cells co-cultured with target T cells expressing cognate neo12 peptide-HLA (right column), but not when co-cultured with target T cells expressing an irrelevant peptide (left column). Tumor cells not displaying the appropriate neoE-HLA target antigen continued to grow in the presence of NeoTCR T cells (left column, green). In contrast, the majority of neo12 peptide displaying tumor cells were apoptotic or dead within 2 days of co-culture with neo12-TCR T cells (right column, red).

Effector function in T cells from healthy donors and patients with cancer. To ensure that the NeoTCR manufacturing process is successful in generating product not only from healthy donors but also from T cells in patients with cancer, ex vivo mechanism-of-action (MOA) studies were performed by generating NeoTCR cells derived from blood obtained from patients with cancer and directly compared to the activity of T cells generated from blood obtained from healthy donors expressing the same NeoTCR. Antigen-specific activity was characterized by mixing NeoTCR-T cells with surrogate tumor target T cells that express cognate or irrelevant HLA-peptide complexes. Comparable gene editing efficiencies and functional activity, as measured by antigen-specificity of T cell killing activity, proliferation, and cytokine production, were observed in studies with NeoTCR-expressing CD8+ and CD4+ T cells from patients with cancer and from healthy donors. No target T cell killing or NeoTCR-T cell proliferation was observed upon contact with target T cells lacking display of the HLA-bound mutated target peptide, thus demonstrating the specificity of the response.

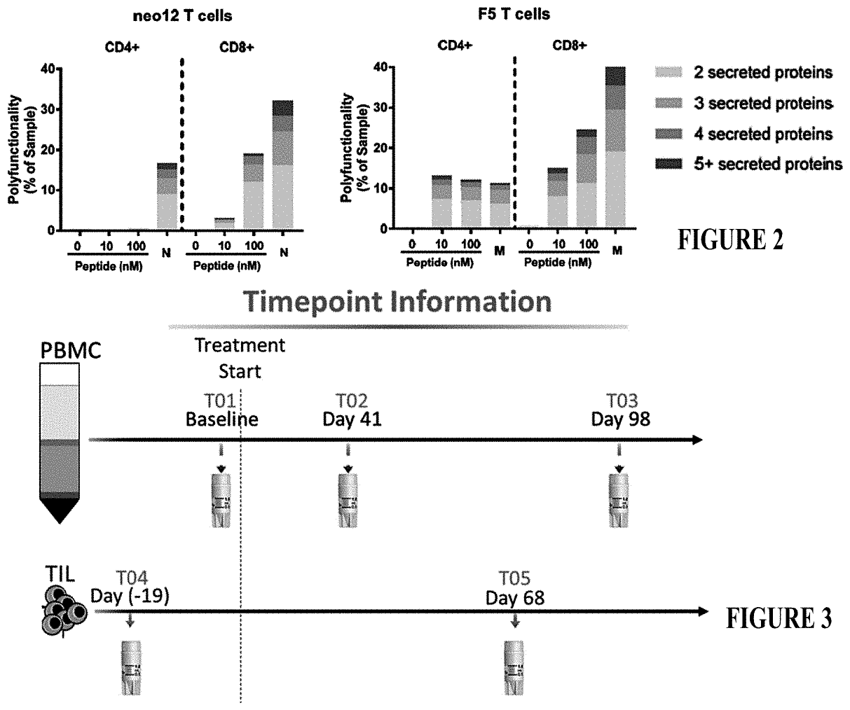

Polyfunctionality of NeoTCR-T cells. In addition to rapidly converting to a functional effector T cell phenotype, T cell polyfunctionality (the ability of a single cell to secrete multiple effector proteins) is a key product attribute that is a highly desirable characteristic among engineered T cells administered in clinical trials. Patients with non-Hodgkin lymphoma dosed with CD19 CAR-T cells exhibiting polyfunctionality prior to infusion were significantly more likely to experience an objective clinical response than patients dosed with engineered CAR-T cells that did not exhibit polyfunctionality (Rossi et al, 2018). Dose-dependent polyfunctional secretion of cytokines from activated CD8+ NeoTCR cells was demonstrated by single cell secretome analysis (FIG. 2). The ratio of different cytokine-producing fractions also appears to be consistent across the stimulation dose response. These data also reveal that CD4 T cells engineered to express an HLA class I-restricted TCR successfully exhibit effector function after exposure to cognate neoE-HLA target. However, for CD4+ T cells, no dose-response effect was observed with the peptide concentrations used to pulse the target T cells. Taken together, these data reveal that a polyfunctional cytokine response is contributed by NeoTCR-expressing CD8 and CD4 T cells upon stimulation with tumor cells displaying the cognate neoE-HLA targets on their surface.

Younger T cell phenotypes. According to the linear model of T cell differentiation, naïve cells differentiate into memory stem cell (T_{MSC}) and central memory (T_{CM}) cell phenotypes upon initial activation with appropriate signals from antigen-presenting cells. These ‘younger’ or less-differentiated T cell populations have been shown to engraft well into lymphocyte-depleted animals, plus to proliferate vigorously on further stimulation while maintaining the memory cell pool for persistence (Klebanoff, Gattinoni and Restifo, 2012). The model for linear differentiation of T cell subsets is presented in Table 6.

TABLE 6
Model for linear differentiation of T cell subsets

| T cell subset | Cytokine profile | Functional characteristics |
|---|---|---|
| Naïve (T_{N}) | CD45RA+CD62L+, | Weak effector T cell |
| | CD28+CD95− | function |
| | CCR7+CD27+ | Robust proliferation |
| Memory stem cell (T_{MSC}) | CD45RA+CD62L+, | Robust engraftment |
| | CD28+CD95+ | Long telomeres |
| | CCR7+CD27+ | |
| Central memory (T_{CM}) | CD45RO+CD62L+, | |
| | CD28+CD95+ | |
| | CCR7+CD27+CD127+ | |
| Effector memory (T_{EM}) | CD45RO+CD62L−, | Rapidly execute effector |
| | CD28+/−CD95+ | functions e.g., IFN-γ, |
| | CCR7−CD27− | TNF-α, lysis of target cells |
| | Busch: CD27+CD127+ | Limited proliferation |
| Effector (T_{E}) | CD45RO+CD62L−, | Low engraftment potential |
| | CD28+/−CD95+ | Short telomere length |
| | Perforin^{hi}Gzmb^{hi} | |
| | CCR7−CD27− | |

These ‘younger’ populations, however, do not secrete the full cytokine and effector protein cascade observed from more differentiated or ‘older’ effector memory (T_{EM}) and effector T cells (T_{E}) upon encountering cognate neoE-HLA expressing targets. However, while the ‘older’ cells are highly effective for killing target tumor cells, they lack the capacity to proliferate and to persist. In this manner, they have become terminally differentiated for the purpose of killing target T cells upon activation.

Published reports from animal models and clinical studies have suggested that adoptive cell therapies comprising T cells with ‘younger’ or less-differentiated memory stem cell phenotypes achieve an improved overall response and clinical outcome than studies where ‘older’ or more-differentiated T effector cells were administered. Data from mouse models and in clinical trials of CD19-targeted CAR-T cells demonstrated a correlation of the younger phenotype with cell persistence and overall response rate (Busch et al, 2018); (Sabatino et al., 2016). Thus, the administration of ‘younger’ T cell phenotypes is of potentially higher benefit to patients with cancer, and this is associated with improved engraftment potential, prolonged persistence post infusion, and rapid differentiation into effector T cells upon exposure to their cognate antigen. These properties highlight the value of infusing NeoTCR-expressing T cells comprising T memory stem cell (T_{MSC}) and T central memory (T_{CM}) phenotypes.

The manufacturing process described herein was deliberately developed to favor the generation of T cell populations of the less-differentiated phenotype. The composition of T cell phenotypes resulting from the NeoTCR Product cell manufacturing process was interrogated by flow cytometric analysis. As desired, NeoTCR cells of memory stem cell and central memory phenotypes represent significant T cell phenotypes in the NeoTCR Product profile.

Together, these ex vivo MOA studies demonstrate that the NeoTCR cells generated from healthy donors or patients with cancer that were formulated into NeoTCR Products comprise CD8+ and CD4+ T cells of the desired younger phenotype subsets (T_{MSC} and T_{CM}). Upon encounter of cognate peptide-HLA, these cells rapidly transition into polyfunctional effector cells that demonstrate potent cytokine production, tumor killing activity, and proliferative capacity, with the potential to eradicate tumor cells throughout the body.

### Example 2. Single and Multi-TCR NeoTCR Product

In certain embodiments, each NeoTCR Product comprises a single NeoTCR that is precision genome engineered into CD4+ and CD8+ T cells. Given the design of the NeoTCR Products to contain a NeoTCR with a truncal mutation, a NeoTCR Product with only one (1) NeoTCR can be sufficient and effective at treating proliferative disorders (e.g., cancer).

There is no executional limit for the number of NeoTCRs that can be included in the NeoTCR Product. Rather, the number of NeoTCRs that can be included in a NeoTCR Product can be selected either 1) based on the number of NeoTCRs identified in a patient tumor and blood sample, or 2) the desire to have multiple NeoTCRs in a given product.

In certain embodiments, the NeoTCR Product comprises a single NeoTCR. NeoTCR Products comprising a single (1) NeoTCR have been made by screening patient tumor and blood samples for NeoTCRs and selecting a single (1) NeoTCR to be engineered into the NeoTCR Product. In certain embodiments, the single (1) NeoTCR that is selected for a 1-NeoTCR Product comprises a truncal mutation. Any single (1) NeoTCR Product can be used to treat cancer using any of the methods described herein.

In certain embodiments, the NeoTCR Product is polyclonal and comprises two (2) or more NeoTCRs. In certain embodiments the polyclonal NeoTCR Product comprises two (2) or more NeoTCRs in a single container. In certain embodiments the polyclonal NeoTCR Product comprises two (2) or more NeoTCRs wherein each of the NeoTCRs are in their own, separate container. In certain embodiments, the NeoTCRs contained in the NeoTCR Product are administered sequentially into a patient for the treatment of cancer. In certain embodiments, the NeoTCRs contained in the NeoTCR Product are administered concurrently into a patient for the treatment of cancer.

In certain embodiments, the NeoTCR Product comprises two (2) or three (3) NeoTCRs. NeoTCR Products comprising two (2) or three (3) NeoTCRs can be made by screening patient tumor and blood samples for NeoTCRs and selecting two (2) or three (3) NeoTCRs to be engineered into the NeoTCR Product. In certain embodiments, the two (2) or three (3) NeoTCRs that are selected for the NeoTCR Product comprise truncal mutations. In certain embodiments, if two (2) NeoTCRs are selected for the NeoTCR Product, both of the two (2) NeoTCRs comprise truncal mutations. In certain embodiments, if three (3) NeoTCRs are selected for the NeoTCR Product, then two (2) of the three (3) NeoTCRs comprise truncal mutations. In certain embodiments, if three (3) NeoTCRs are selected for the NeoTCR Product, then all three (3) NeoTCRs comprise truncal mutations. Any two (2) or three (3) NeoTCR Product can be used to treat cancer using any of the methods described herein.

In certain embodiments, the NeoTCR Product comprises four (4) NeoTCRs. NeoTCR Products comprising four (4) NeoTCRs can be made by screening patient tumor and blood samples for NeoTCRs and selecting four (4) NeoTCRs to be engineered into the NeoTCR Product. In certain embodiments, the four (4) NeoTCRs that are selected for the NeoTCR Product comprise truncal mutations. In certain embodiments, if four (4) NeoTCRs are selected for the NeoTCR Product, all four (4) NeoTCRs comprise truncal mutations. In certain embodiments, if four (4) NeoTCRs are selected for the NeoTCR Product, then two (2) of the four (4) NeoTCRs comprise truncal mutations. In certain embodiments, if four (4) NeoTCRs are selected for the NeoTCR Product, then three (3) of the four (4) NeoTCRs comprise truncal mutations. Any four (4) NeoTCR Product can be used to treat cancer using any of the methods described herein.

In certain embodiments, the NeoTCR Product comprises five (5) NeoTCRs. NeoTCR Products comprising five (5) can be made by screening patient tumor and blood samples for NeoTCRs and selecting five (5) NeoTCRs to be engineered into the NeoTCR Product. In certain embodiments, the five (5) NeoTCRs that are selected for the NeoTCR Product comprise truncal mutations. In certain embodiments, if five (5) NeoTCRs are selected for the NeoTCR Product, all five (5) NeoTCRs comprise truncal mutations. In certain embodiments, if five (5) NeoTCRs are selected for the NeoTCR Product, then two (2) of the five (5) NeoTCRs comprise truncal mutations. In certain embodiments, if five (5) NeoTCRs are selected for the NeoTCR Product, then three (3) of the five (5) NeoTCRs comprise truncal mutations. In certain embodiments, if five (5) NeoTCRs are selected for the NeoTCR Product, then four (4) of the five (5) NeoTCRs comprise truncal mutations. Any five (5) NeoTCR Product can be used to treat cancer using any of the methods described herein.

In certain embodiments, the NeoTCR Product comprises six (6) NeoTCRs. NeoTCR Products comprising six (6) can be made by screening patient tumor and blood samples for NeoTCRs and selecting six (6) NeoTCRs to be engineered into the NeoTCR Product. In certain embodiments, the six (6) NeoTCRs that are selected for the NeoTCR Product comprise truncal mutations. In certain embodiments, if six (6) NeoTCRs are selected for the NeoTCR Product, all six (6) NeoTCRs comprise truncal mutations. In certain embodiments, if six (6) NeoTCRs are selected for the NeoTCR Product, then two (2) of the six (6) NeoTCRs comprise truncal mutations. In certain embodiments, if six (6) NeoTCRs are selected for the NeoTCR Product, then three (3) of the six (6) NeoTCRs comprise truncal mutations. In certain embodiments, if six (6) NeoTCRs are selected for the NeoTCR Product, then four (4) of the six (6) NeoTCRs comprise truncal mutations. In certain embodiments, if six (6) NeoTCRs are selected for the NeoTCR Product, then five (5) of the six (6) NeoTCRs comprise truncal mutations. Any six (6) NeoTCR Product can be used to treat cancer using any of the methods described herein.

In certain embodiments, the NeoTCR Product comprises seven (7) NeoTCRs. NeoTCR Products comprising seven (7) can be made by screening patient tumor and blood samples for NeoTCRs and selecting seven (7) NeoTCRs to be engineered into the NeoTCR Product. In certain embodiments, the seven (7) NeoTCRs that are selected for the NeoTCR Product comprise truncal mutations. In certain embodiments, if seven (7) NeoTCRs are selected for the NeoTCR Product, all seven (7) NeoTCRs comprise truncal mutations. In certain embodiments, if seven (7) NeoTCRs are selected for the NeoTCR Product, then two (2) of the seven (7) NeoTCRs comprise truncal mutations. In certain embodiments, if seven (7) NeoTCRs are selected for the NeoTCR Product, then three (3) of the seven (7) NeoTCRs comprise truncal mutations. In certain embodiments, if seven (7) NeoTCRs are selected for the NeoTCR Product, then four (4) of the seven (7) NeoTCRs comprise truncal mutations. In certain embodiments, if seven (7) NeoTCRs are selected for the NeoTCR Product, then five (5) of the seven (7) NeoTCRs comprise truncal mutations. In certain embodiments, if seven (7) NeoTCRs are selected for the NeoTCR Product, then six (6) of the seven (7) NeoTCRs comprise truncal mutations. Any seven (7) NeoTCR Product can be used to treat cancer using any of the methods described herein.

In certain embodiments, the NeoTCR Product comprises eight (8) NeoTCRs. NeoTCR Products comprising eight (8) can be made by screening patient tumor and blood samples for NeoTCRs and selecting eight (8) NeoTCRs to be engineered into the NeoTCR Product. In certain embodiments, the eight (8) NeoTCRs that are selected for the NeoTCR Product comprise truncal mutations. In certain embodiments, if eight (8) NeoTCRs are selected for the NeoTCR Product, all eight (8) NeoTCRs comprise truncal mutations. In certain embodiments, if eight (8) NeoTCRs are selected for the NeoTCR Product, then two (2) of the eight (8) NeoTCRs comprise truncal mutations. In certain embodiments, if eight (8) NeoTCRs are selected for the NeoTCR Product, then three (3) of the eight (8) NeoTCRs comprise truncal mutations. In certain embodiments, if eight (8) NeoTCRs are selected for the NeoTCR Product, then four (4) of the eight (8) NeoTCRs comprise truncal mutations. In certain embodiments, if eight (8) NeoTCRs are selected for the NeoTCR Product, then five (5) of the eight (8) NeoTCRs comprise truncal mutations. In certain embodiments, if eight (8) NeoTCRs are selected for the NeoTCR Product, then six (6) of the eight (8) NeoTCRs comprise truncal mutations. In certain embodiments, if eight (8) NeoTCRs are selected for the NeoTCR Product, then seven (7) of the eight (8) NeoTCRs comprise truncal mutations. Any eight (8) NeoTCR Product can be used to treat cancer using any of the methods described herein.

In certain embodiments, the NeoTCR Product comprises nine (9) NeoTCRs. NeoTCR Products comprising nine (9) can be made by screening patient tumor and blood samples for NeoTCRs and selecting nine (9) NeoTCRs to be engineered into the NeoTCR Product. In certain embodiments, the nine (9) NeoTCRs that are selected for the NeoTCR Product comprise truncal mutations. In certain embodiments, if nine (9) NeoTCRs are selected for the NeoTCR Product, all nine (9) NeoTCRs comprise truncal mutations. In certain embodiments, if nine (9) NeoTCRs are selected for the NeoTCR Product, then two (2) of the nine (9) NeoTCRs comprise truncal mutations. In certain embodiments, if nine (9) NeoTCRs are selected for the NeoTCR Product, then three (3) of the nine (9) NeoTCRs comprise truncal mutations. In certain embodiments, if nine (9) NeoTCRs are selected for the NeoTCR Product, then four (4) of the nine (9) NeoTCRs comprise truncal mutations. In certain embodiments, if nine (9) NeoTCRs are selected for the NeoTCR Product, then five (5) of the nine (9) NeoTCRs comprise truncal mutations. In certain embodiments, if nine (9) NeoTCRs are selected for the NeoTCR Product, then six (6) of the nine (9) NeoTCRs comprise truncal mutations. In certain embodiments, if nine (9) NeoTCRs are selected for the NeoTCR Product, then seven (7) of the nine (9) NeoTCRs comprise truncal mutations. In certain embodiments, if nine (9) NeoTCRs are selected for the NeoTCR Product, then eight (8) of the nine (9) NeoTCRs comprise truncal mutations. Any nine (9) NeoTCR Product can be used to treat cancer using any of the methods described herein.

In certain embodiments, the NeoTCR Product comprises ten (10) NeoTCRs. NeoTCR Products comprising ten (10) can be made by screening patient tumor and blood samples for NeoTCRs and selecting ten (10) NeoTCRs to be engineered into the NeoTCR Product. In certain embodiments, the ten (10) NeoTCRs that are selected for the NeoTCR Product comprise truncal mutations. In certain embodiments, if ten (10) NeoTCRs are selected for the NeoTCR Product, all ten (10) NeoTCRs comprise truncal mutations. In certain embodiments, if ten (10) NeoTCRs are selected for the NeoTCR Product, then two (2) of the ten (10) NeoTCRs comprise truncal mutations. In certain embodiments, if ten (10) NeoTCRs are selected for the NeoTCR Product, then three (3) of the ten (10) NeoTCRs comprise truncal mutations. In certain embodiments, if ten (10) NeoTCRs are selected for the NeoTCR Product, then four (4) of the ten (10) NeoTCRs comprise truncal mutations. In certain embodiments, if ten (10) NeoTCRs are selected for the NeoTCR Product, then five (5) of the ten (10) NeoTCRs comprise truncal mutations. In certain embodiments, if ten (10) NeoTCRs are selected for the NeoTCR Product, then six (6) of the ten (10) NeoTCRs comprise truncal mutations. In certain embodiments, if ten (10) NeoTCRs are selected for the NeoTCR Product, then seven (7) of the ten (10) NeoTCRs comprise truncal mutations. In certain embodiments, if ten (10) NeoTCRs are selected for the NeoTCR Product, then eight (8) of the ten (10) NeoTCRs comprise truncal mutations. In certain embodiments, if ten (10) NeoTCRs are selected for the NeoTCR Product, then nine (9) of the ten (10) NeoTCRs comprise truncal mutations. Any ten (10) NeoTCR Product can be used to treat cancer using any of the methods described herein.

In certain embodiments, the NeoTCR Product comprises eleven (11) NeoTCRs. NeoTCR Products comprising eleven (11) can be made by screening patient tumor and blood samples for NeoTCRs and selecting eleven (11) NeoTCRs to be engineered into the NeoTCR Product. In certain embodiments, the eleven (11) NeoTCRs that are selected for the NeoTCR Product comprise truncal mutations. In certain embodiments, one or more of the eleven (11) NeoTCRs comprise truncal mutations. Any eleven (11) NeoTCR Product can be used to treat cancer using any of the methods described herein.

In certain embodiments, the NeoTCR Product comprises twelve (12) NeoTCRs. NeoTCR Products comprising twelve (12) can be made by screening patient tumor and blood samples for NeoTCRs and selecting twelve (12) NeoTCRs to be engineered into the NeoTCR Product. In certain embodiments, the twelve (12) NeoTCRs that are selected for the NeoTCR Product comprise truncal mutations. In certain embodiments, one or more of the twelve (12) NeoTCRs comprise truncal mutations. Any twelve (12) NeoTCR Product can be used to treat cancer using any of the methods described herein.

In certain embodiments, the NeoTCR Product comprises thirteen (13) NeoTCRs. NeoTCR Products comprising thirteen (13) can be made by screening patient tumor and blood samples for NeoTCRs and selecting thirteen (13) NeoTCRs to be engineered into the NeoTCR Product. In certain embodiments, the thirteen (13) NeoTCRs that are selected for the NeoTCR Product comprise truncal mutations. In certain embodiments, one or more of the thirteen (13) NeoTCRs comprise truncal mutations. Any thirteen (13) NeoTCR Product can be used to treat cancer using any of the methods described herein.

In certain embodiments, the NeoTCR Product comprises fourteen (14) NeoTCRs. NeoTCR Products comprising fourteen (14) can be made by screening patient tumor and blood samples for NeoTCRs and selecting fourteen (14) NeoTCRs to be engineered into the NeoTCR Product. In certain embodiments, the fourteen (14) NeoTCRs that are selected for the NeoTCR Product comprise truncal mutations. In certain embodiments, one or more of the fourteen (14) NeoTCRs comprise truncal mutations. Any fourteen (14) NeoTCR Product can be used to treat cancer using any of the methods described herein.

In certain embodiments, the NeoTCR Product comprises fifteen (15) NeoTCRs. NeoTCR Products comprising fifteen (15) can be made by screening patient tumor and blood samples for NeoTCRs and selecting fifteen (15) NeoTCRs to be engineered into the NeoTCR Product. In certain embodiments, the fifteen (15) NeoTCRs that are selected for the NeoTCR Product comprise truncal mutations. In certain embodiments, one or more of the fifteen (15) NeoTCRs comprise truncal mutations. Any fifteen (15) NeoTCR Product can be used to treat cancer using any of the methods described herein.

In certain embodiments, the NeoTCR Product comprises sixteen (16) NeoTCRs. NeoTCR Products comprising sixteen (16) can be made by screening patient tumor and blood samples for NeoTCRs and selecting sixteen (16) NeoTCRs to be engineered into the NeoTCR Product. In certain embodiments, the sixteen (16) NeoTCRs that are selected for the NeoTCR Product comprise truncal mutations. In certain embodiments, one or more of the sixteen (16) NeoTCRs comprise truncal mutations. Any sixteen (16) NeoTCR Product can be used to treat cancer using any of the methods described herein.

In certain embodiments, the NeoTCR Product comprises seventeen (17) NeoTCRs. NeoTCR Products comprising seventeen (17) can be made by screening patient tumor and blood samples for NeoTCRs and selecting seventeen (17) NeoTCRs to be engineered into the NeoTCR Product. In certain embodiments, the seventeen (17) NeoTCRs that are selected for the NeoTCR Product comprise truncal mutations. In certain embodiments, one or more of the seventeen (17) NeoTCRs comprise truncal mutations. Any seventeen (17) NeoTCR Product can be used to treat cancer using any of the methods described herein.

In certain embodiments, the NeoTCR Product comprises eighteen (18) NeoTCRs. NeoTCR Products comprising eighteen (18) can be made by screening patient tumor and blood samples for NeoTCRs and selecting eighteen (18) NeoTCRs to be engineered into the NeoTCR Product. In certain embodiments, the eighteen (18) NeoTCRs that are selected for the NeoTCR Product comprise truncal mutations. In certain embodiments, one or more of the eighteen (18) NeoTCRs comprise truncal mutations. Any eighteen (18) NeoTCR Product can be used to treat cancer using any of the methods described herein.

In certain embodiments, the NeoTCR Product comprises nineteen (19) NeoTCRs. NeoTCR Products comprising nineteen (19) can be made by screening patient tumor and blood samples for NeoTCRs and selecting nineteen (19) NeoTCRs to be engineered into the NeoTCR Product. In certain embodiments, the nineteen (19) NeoTCRs that are selected for the NeoTCR Product comprise truncal mutations. In certain embodiments, one or more of the nineteen (19) NeoTCRs comprise truncal mutations. Any nineteen (19) NeoTCR Product can be used to treat cancer using any of the methods described herein.

In certain embodiments, the NeoTCR Product comprises twenty (20) NeoTCRs. NeoTCR Products comprising twenty (20) can be made by screening patient tumor and blood samples for NeoTCRs and selecting twenty (20) NeoTCRs to be engineered into the NeoTCR Product. In certain embodiments, the twenty (20) NeoTCRs that are selected for the NeoTCR Product comprise truncal mutations. In certain embodiments, one or more of the twenty (20) NeoTCRs comprise truncal mutations. Any twenty (20) NeoTCR Product can be used to treat cancer using any of the methods described herein.

In certain embodiments, the NeoTCR Product comprises twenty-one (21) or more NeoTCRs. NeoTCR Products comprising twenty-one (21) or more can be made by screening patient tumor and blood samples for NeoTCRs and selecting twenty-one (21) or more NeoTCRs to be engineered into the NeoTCR Product. In certain embodiments, the twenty-one (21) or more NeoTCRs that are selected for the NeoTCR Product comprise truncal mutations. In certain embodiments, one or more of the twenty-one (21) or more NeoTCRs comprise truncal mutations. Any twenty-one (21) or more NeoTCR Product can be used to treat cancer using any of the methods described herein.

In certain embodiments, the number of NeoTCRs included in a NeoTCR Product are limited only by the number of NeoTCRs detected by screening patient tumor and blood samples. In certain embodiments, every NeoTCR detected by screening patient tumor and blood samples is included in the NeoTCR Product.

### Example 3. NeoTCR Product Combination Therapy

Summary.

The NeoTCR Products can be administered alone or they can be used in a combination therapy. The combination therapy can include administering NeoTCR Product and administering 1, 2, 3, 4, 5, 6, or 7 or more additional therapeutic agent. In certain embodiments, the combination therapy includes administering NeoTCR Product and administering at least one additional therapeutic agent. In certain embodiments, the combination therapy includes administering NeoTCR Product and administering at least two additional therapeutic agents. In certain embodiments, the combination therapy includes administering NeoTCR Product and administering at least three additional therapeutic agents. In certain embodiments, the combination therapy includes administering NeoTCR Product and administering at least four additional therapeutic agents. In certain embodiments, the combination therapy includes administering NeoTCR Product and administering at least five additional therapeutic agents. In certain embodiments, the combination therapy includes administering NeoTCR Product and administering at least six additional therapeutic agents. In certain embodiments, the combination therapy includes administering NeoTCR Product and administering at least seven additional therapeutic agents.

In any of the embodiments provided above, the NeoTCR Product can be a single NeoTCR Product or two or more NeoTCR Products which are optionally administered simultaneously or consecutively with one another.

In any of the embodiments provided above, the NeoTCR Product can contain a single NeoTCR or two or more NeoTCRs.

In certain embodiments, the NeoTCR Product is administered simultaneously with the one or more combination therapies. In certain embodiments, the NeoTCR Product and the one or more combination therapies are administered consecutively.

In certain embodiments, the combination agent is a chemotherapeutic agent. In certain embodiments, the combination agent is two (2) chemotherapeutic agents. In certain embodiments, the combination agent is three (3) chemotherapeutic agents. In certain embodiments, the combination agent is four (4) or more chemotherapeutic agents.

In certain embodiments, the combination agent is radiation therapy.

In certain embodiments, the chemotherapeutic agent(s) is/are hormonal therapy(ies).

In certain embodiments, the chemotherapeutic agent(s) is/are immunotherapy(ies).

In certain embodiments, the chemotherapeutic agent(s) is/are platinum-based antineoplastic agent(s). In certain embodiments, the chemotherapeutic agent(s) is/are alkylating agent(s). In certain embodiments, the chemotherapeutic agent(s) is/are platinum-based agent(s). In certain embodiments, the platinum-based agents are selected from the consisting of cisplatin, carboplatin, oxaliplatin. In certain embodiments, the chemotherapeutic agent(s) is/are microtubule inhibitor(s). In certain embodiments, the chemotherapeutic agent(s) is/are purine analogue(s).

PD-1 Axis Binding Agents.

Modulation of the immunosuppressive tumor microenvironment with immune-checkpoint blockade is a promising strategy to bolster the potency of adoptively transferred T cells. Results of preclinical studies in numerous mouse models have demonstrated that combining adoptive T cell therapy with PD1 pathway blockade can improve tumor burden control and can prolong median survival (Cherkassky, et al., 2016) (Moon et al, 2015) (Kodumudi et al, 2016) (Rupp et al, 2017). Blockade of PD-1/PD-L1 signaling restores the function of PD-1-expressing T cells that encounter PD-L1, thus decreasing T cell susceptibility to tumor-induced hypofunction (Gerner et al, 2013). By enhancing the proliferation of transferred T cells, PD1 inhibition can also increase IFNγ levels in the tumor microenvironment, leading to increased CXCL10 production and the recruitment of more tumor-reactive T cells (Peng et al, 2012). Importantly, the benefit of combining PD1/PD-L1 blockade and adoptively transferred tumor infiltrating lymphocytes (TIL) has been observed in preclinical tumor models and in clinical settings (Goff et al, 2016). Together, the literature studies support the premise that PD1/PD-L1 inhibition will enhance anti-tumor immune responses after adoptive transfer of NeoTCR Product and provides a strong rationale for pursuing this combination therapy in the clinic.

In certain embodiments, the combination agent is a PD-1 binding agent. In certain embodiments, the PD-1 binding agent is pembrolizumab. In certain embodiments, the PD-1 binding agent is nivolumab. In certain embodiments, the PD-1 binding agent is any other PD-1 binding agent described herein.

In certain embodiments, the combination agent is a PD-L1 binding agent. In certain embodiments, the PD-L1 binding agent is atezolizumab. In certain embodiments, the PD-L1 binding agent is any other PD-L1 binding agent described herein.

In certain embodiments, the combination agent is a PD-L2 binding agent.

In certain embodiments, the PD-1 axis binding agent is administered prior to the administration of the NeoTCR Product. In certain embodiments, the PD-1 axis binding agent is administered after the administration of the NeoTCR Product. In certain embodiments, the PD-1 axis binding agent is administered at the same time as the NeoTCR Product.

In certain embodiments, the PD-1 axis binding agent is administered on the same day as the NeoTCR Product. In certain embodiments, the PD-1 axis binding agent is administered on consecutive days as the NeoTCR Product (e.g., if the PD-1 axis binding agent is administered on a Monday then the NeoTCR Product is administered one day later on Tuesday; if the NeoTCR Product is administered on a Monday then the PD-1 axis binding agent is administered one day later on Tuesday). In certain embodiments, the PD-1 axis binding agent is administered two (2), three (3), four (4), five (5), six (6) or seven (7) days after the NeoTCR Product is administered. In certain embodiments, the NeoTCR Product is administered two (2), three (3), four (4), five (5), six (6) or seven (7) days after the PD-1 axis binding agent is administered. In certain embodiments, the PD-1 axis binding agent is administered two (2) weeks after the NeoTCR Product is administered. In certain embodiments, the NeoTCR Product is administered two (2) weeks after the PD-1 axis binding agent is administered. In certain embodiments, the PD-1 axis binding agent is administered one (1) month after the NeoTCR Product is administered. In certain embodiments, the NeoTCR Product is administered one (1) month after the PD-1 axis binding agent is administered.

Cytokines.

In certain embodiments, cytokines (and derivatives/modifications thereof) with therapeutic benefits for the treatment of cancer can be combined with NeoTCR Products.

IL-2 is a cytokine that does not kill cancer cells directly. IL-2 Agents includes 1) IL-2 and modifications and derivatives thereof that like native IL-2 does not kill cancer cells and 2) IL-2 molecules that have been engineered and modified to directly kill cancer cells. The native IL-2 and native IL-2 component(s) of IL-2 Agents work by stimulating certain cells in a patient's immune system to kill the cancer cells. Because cancer cells are capable of evading the immune system (i.e., the immune system does not recognize the cancer cells as abnormal or dangerous), IL-2 Agents can be administered to patients in need thereof to boost the immune system to kill, reduce the proliferation of, or stop the proliferation of the cancer cells.

In certain embodiments, the IL-2 Agent is IL-2 or a pharmaceutically acceptable formulation thereof. In certain embodiments, the pharmaceutically acceptable formulation of IL-2 is aldesleukin.

In certain embodiments, the IL-2 Agent is a CD122-preferential IL-2 pathway agonist.

In certain embodiments, the IL-2 Agent is a pegylated IL-2. In certain embodiments, the pegylated IL-2 is NKTR-214 (bempegaldesleukin).

In certain embodiments, the IL-2 Agent is an engineered IL-2 variant (IL2v). In certain embodiments, the IL2v is engineered with an antibody against fibroblast activation protein (FAP). In certain embodiments, the IL2v is RG7461.

In certain embodiments, the IL-2 Agent is an engineered anti-CEA antibody that contains an IL2v moiety. In certain embodiments, the anti-CEA antibody that contains an IL2v moiety is cergutuzumab amunaleukin.

In certain embodiments, the IL-2 Agent is administered prior to the administration of the NeoTCR Product. In certain embodiments, the IL-2 Agent is administered after the administration of the NeoTCR Product. In certain embodiments, the IL-2 Agent is administered at the same time as the NeoTCR Product.

In certain embodiments, the IL-2 Agent is administered on the same day as the NeoTCR Product. In certain embodiments, the IL-2 Agent is administered on consecutive days as the NeoTCR Product (e.g., if the IL-2 Agent is administered on a Monday then the NeoTCR Product is administered one day later on Tuesday; if the NeoTCR Product is administered on a Monday then the IL-2 Agent is administered one day later on Tuesday). In certain embodiments, the IL-2 Agent is administered two (2), three (3), four (4), five (5), six (6) or seven (7) days after the NeoTCR Product is administered. In certain embodiments, the NeoTCR Product is administered two (2), three (3), four (4), five (5), six (6) or seven (7) days after the IL-2 Agent is administered. In certain embodiments, the IL-2 Agent is administered two (2) weeks after the NeoTCR Product is administered. In certain embodiments, the NeoTCR Product is administered two (2) weeks after the IL-2 Agent is administered. In certain embodiments, the IL-2 Agent is administered one (1) month after the NeoTCR Product is administered. In certain embodiments, the NeoTCR Product is administered one (1) month after the IL-2 Agent is administered.

In certain embodiments, IL-10 agents or a pharmaceutically acceptable formulation thereof can be combined with one or more NeoTCR Product. In certain embodiments, the IL-10 agent is pegilodecakin.

In certain embodiments, the IL-10 Agent is administered prior to the administration of the NeoTCR Product. In certain embodiments, the IL-10 Agent is administered after the administration of the NeoTCR Product. In certain embodiments, the IL-10 Agent is administered at the same time as the NeoTCR Product.

In certain embodiments, the IL-10 Agent is administered on the same day as the NeoTCR Product. In certain embodiments, the IL-10 Agent is administered on consecutive days as the NeoTCR Product (e.g., if the IL-10 Agent is administered on a Monday then the NeoTCR Product is administered one day later on Tuesday; if the NeoTCR Product is administered on a Monday then the IL-10 Agent is administered one day later on Tuesday). In certain embodiments, the IL-10 Agent is administered two (2), three (3), four (4), five (5), six (6) or seven (7) days after the NeoTCR Product is administered. In certain embodiments, the NeoTCR Product is administered two (2), three (3), four (4), five (5), six (6) or seven (7) days after the IL-10 Agent is administered. In certain embodiments, the IL-10 Agent is administered two (2) weeks after the NeoTCR Product is administered. In certain embodiments, the NeoTCR Product is administered two (2) weeks after the IL-10 Agent is administered. In certain embodiments, the IL-10 Agent is administered one (1) month after the NeoTCR Product is administered. In certain embodiments, the NeoTCR Product is administered one (1) month after the IL-10 Agent is administered.

In certain embodiments, an IL-15 Agent can be combined with one or more NeoTCR Products. In certain embodiments, an IL-15 Agent that enhances formation of long-term immunological memory which leads to sustained anti-tumor immune response can be combined with one or more NeoTCR Products.

In certain embodiments, the IL-15 Agent targets the IL-15α/IL-2Rγ receptor complex. In certain embodiments, the IL-15 Agent is NKTR-255.

In certain embodiments, the IL-15 Agent is a native IL-15. In certain embodiments, the IL-15 Agent comprises a sushi domain. In certain embodiments, the IL-15 Agent is a potency-reduced IL15-IL15Rα heterodimeric Fc-fusion. In certain embodiments, the IL-15 Agent is P22339. In certain embodiments, the IL-15 Agent is XmAb24306.

In certain embodiments, the IL-15 Agent is administered prior to the administration of the NeoTCR Product. In certain embodiments, the IL-15 Agent is administered after the administration of the NeoTCR Product. In certain embodiments, the IL-15 Agent is administered at the same time as the NeoTCR Product.

In certain embodiments, the IL-15 Agent is administered on the same day as the NeoTCR Product. In certain embodiments, the IL-15 Agent is administered on consecutive days as the NeoTCR Product (e.g., if the IL-15 Agent is administered on a Monday then the NeoTCR Product is administered one day later on Tuesday; if the NeoTCR Product is administered on a Monday then the IL-15 Agent is administered one day later on Tuesday). In certain embodiments, the IL-15 Agent is administered two (2), three (3), four (4), five (5), six (6) or seven (7) days after the NeoTCR Product is administered. In certain embodiments, the NeoTCR Product is administered two (2), three (3), four (4), five (5), six (6) or seven (7) days after the IL-15 Agent is administered. In certain embodiments, the IL-15 Agent is administered two (2) weeks after the NeoTCR Product is administered. In certain embodiments, the NeoTCR Product is administered two (2) weeks after the IL-15 Agent is administered. In certain embodiments, the IL-15 Agent is administered one (1) month after the NeoTCR Product is administered. In certain embodiments, the NeoTCR Product is administered one (1) month after the IL-15 Agent is administered.

In certain embodiments, an IL-7 Agent can be combined with one or more NeoTCR Products.

In certain embodiments, an IL-12 Agent can be combined with one or more NeoTCR Products.

In certain embodiments, an IL-18 Agent can be combined with one or more NeoTCR Products.

In certain embodiments, an IL-21 Agent can be combined with one or more NeoTCR Products.

Cytokines and PD-1 Axis Binding Agents.

In certain embodiments, one or more NeoTCR Products can be combined with 1) a cytokine (and modifications/derivatives thereof) with therapeutic benefits for the treatment of cancer and, 2) a PD-1 Axis Binding Agent.

In certain embodiments, one or more NeoTCR Products can be combined with an IL-2 Agent and a PD-1 binding Agent. In certain embodiments, one or more NeoTCR Products can be combined with an IL-2 Agent and a PD-L1 binding Agent. In certain embodiments, one or more NeoTCR Products can be combined with an IL-2 Agent and a PD-L2 binding Agent.

In certain embodiments, one or more NeoTCR Products can be combined with an IL-2 Agent and pembrolizumab. In certain embodiments, one or more NeoTCR Products can be combined with an IL-2 Agent and nivolumab. In certain embodiments, one or more NeoTCR Products can be combined with an IL-2 Agent and atezolizumab.

By means of non-limiting examples, in certain embodiments one or more NeoTCR Products can be combined with aldesleukin and pembrolizumab. In certain embodiments, one or more NeoTCR Products can be combined with aldesleukin and nivolumab. In certain embodiments, one or more NeoTCR Products can be combined with aldesleukin and atezolizumab.

By means of non-limiting examples, in certain embodiments one or more NeoTCR Products can be combined with cergutuzumab amunaleukin and pembrolizumab. In certain embodiments, one or more NeoTCR Products can be combined with cergutuzumab amunaleukin and nivolumab. In certain embodiments, one or more NeoTCR Products can be combined with cergutuzumab amunaleukin and atezolizumab.

By means of non-limiting examples, in certain embodiments one or more NeoTCR Products can be combined with bempegaldesleukin and pembrolizumab. In certain embodiments, one or more NeoTCR Products can be combined with bempegaldesleukin and nivolumab. In certain embodiments, one or more NeoTCR Products can be combined with bempegaldesleukin and atezolizumab.

By means of non-limiting examples, in certain embodiments one or more NeoTCR Products can be combined with an IL2v and pembrolizumab. In certain embodiments, one or more NeoTCR Products can be combined with an IL2v and nivolumab. In certain embodiments, one or more NeoTCR Products can be combined with an IL2v and atezolizumab. In certain embodiments, the IL2v is engineered with an antibody against fibroblast activation protein (FAP). In certain embodiments, the IL2v is RG7461.

In certain embodiments, one or more NeoTCR Products can be combined with an IL-10 Agent and a PD-1 binding Agent. In certain embodiments, one or more NeoTCR Products can be combined with an IL-10 Agent and a PD-L1 binding Agent. In certain embodiments, one or more NeoTCR Products can be combined with an IL-10 Agent and a PD-L2 binding Agent.

In certain embodiments, one or more NeoTCR Products can be combined with an IL-10 Agent and pembrolizumab. In certain embodiments, one or more NeoTCR Products can be combined with an IL-10 Agent and nivolumab. In certain embodiments, one or more NeoTCR Products can be combined with an IL-10 Agent and atezolizumab.

By means of non-limiting examples, in certain embodiments one or more NeoTCR Products can be combined with pegilodecakin and pembrolizumab. In certain embodiments, one or more NeoTCR Products can be combined with pegilodecakin and nivolumab. In certain embodiments, one or more NeoTCR Products can be combined with pegilodecakin and atezolizumab.

In certain embodiments, one or more NeoTCR Products can be combined with an IL-15 Agent and a PD-1 binding Agent. In certain embodiments, one or more NeoTCR Products can be combined with an IL-15 Agent and a PD-L1 binding Agent. In certain embodiments, one or more NeoTCR Products can be combined with an IL-15 Agent and a PD-L2 binding Agent.

In certain embodiments, one or more NeoTCR Products can be combined with an IL-15 Agent and pembrolizumab. In certain embodiments, one or more NeoTCR Products can be combined with an IL-15 Agent and nivolumab. In certain embodiments, one or more NeoTCR Products can be combined with an IL-15 Agent and atezolizumab.

By means of non-limiting examples, in certain embodiments one or more NeoTCR Products can be combined with NKTR-255 and pembrolizumab. In certain embodiments, one or more NeoTCR Products can be combined with NKTR-255 and nivolumab. In certain embodiments, one or more NeoTCR Products can be combined with NKTR-255 and atezolizumab.

By means of non-limiting examples, in certain embodiments one or more NeoTCR Products can be combined with an XmAb24306 and pembrolizumab. In certain embodiments, one or more NeoTCR Products can be combined with an XmAb24306 and nivolumab. In certain embodiments, one or more NeoTCR Products can be combined with an XmAb24306 and atezolizumab.

VEGF Agents

In certain embodiments, agents that target VEGF and pharmaceutically acceptable formations thereof can be combined with one or more NeoTCR Products.

In certain embodiments, the agent that targets VEGF is an anti-VEGF antibody. In certain embodiments, the anti-VEGF antibody is bevacizumab. In certain embodiments, the anti-VEGF antibody is ranibizumab. In certain embodiments, the anti-VEGF antibody is ramucirumab.

In certain embodiments, the agent that targets VEGF is an anti-VEGF small molecule. In certain embodiments, the anti-VEGF small molecule is pegaptanib. In certain embodiments, the anti-VEGF small molecule is sorafenib. In certain embodiments, the anti-VEGF small molecule is sunitinib. In certain embodiments, the anti-VEGF small molecule is pazopanib. In certain embodiments, the anti-VEGF small molecule is zif-aflibercept. In certain embodiments, the anti-VEGF small molecule is vandetanib. In certain embodiments, the anti-VEGF small molecule is apatinib. In certain embodiments, the anti-VEGF small molecule is cabozantinib.

In certain embodiments, agents that target VEGF (including any of the anti-VEGF agents disclosed herein) and pharmaceutically acceptable formations thereof can be combined with 1) one or more NeoTCR Products, and 2) a cytokine (and modifications/derivatives thereof).

In certain embodiments, agents that target VEGF (including any of the anti-VEGF agents disclosed herein) and pharmaceutically acceptable formations thereof can be combined with 1) one or more NeoTCR Products, and 2) a PD-1 Axis Binding Agent.

In certain embodiments, agents that target VEGF (including any of the anti-VEGF agents disclosed herein) and pharmaceutically acceptable formations thereof can be combined with 1) one or more NeoTCR Products, 2) a cytokine (and modifications/derivatives thereof), and 3) a PD-1 Axis Binding Agent.

In certain embodiments, agents that target VEGF (including any of the anti-VEGF agents disclosed herein) and pharmaceutically acceptable formations thereof can be combined with 1) one or more NeoTCR Products, 2) optionally a cytokine (and modifications/derivatives thereof), 3) optionally a PD-1 Axis Binding Agent, and 4) another chemotherapeutic agent.

CTLA-4 Agents

In certain embodiments, agents that target CTLA-4 (including but not limited to anti-CTLA-4 antibodies) and pharmaceutically acceptable formations thereof can be combined with one or more NeoTCR Products.

In certain embodiments, the anti-CTLA-4 antibody is ipilimumab. In certain embodiments, the anti-CTLA-4 antibody is a CTLA-4-Fc fusion protein. In certain embodiments, the anti-CTLA-4 antibody is a bispecific antibody targeting CTLA-4 and PD-1. In certain embodiments, the anti-CTLA-4 antibody is a bispecific antibody targeting CTLA-4 and PD-L1.

TIGIT Agents

In certain embodiments, agents that target TIGIT (including but not limited to anti-TIGIT antibodies) and pharmaceutically acceptable formations thereof can be combined with one or more NeoTCR Products.

4-1BB Agents

In certain embodiments, agents that target 4-1BB (including but not limited to anti-4-1BB antibodies) and pharmaceutically acceptable formations thereof can be combined with one or more NeoTCR Products.

In certain embodiments, the 4-1BB agent can be a FAP-4-1BB bispecific antibody. In certain embodiments, the 4-1BB agent can be any bispecific antibody that targets 4-1BB and a tumor surface antigen. In certain embodiments, the 4-1BB agent can be any multispecific antibody that targets 4-1BB, an NK cell maker, and optionally a tumor surface antigen. A non-limiting example of a multispecific 4-1BB antibody is a bispecific antibody that binds to CD19 and 4-1BB.

In certain embodiments, the 4-1BB agent can be a CAR T cell therapy. In certain embodiments, the CAR T cell therapy comprises CD19-targeted CAR T cells with a 4-1BB costimulatory domain.

Carcinoembryonic antigen (CEA) Agents

In certain embodiments, agents that target CEA (including but not limited to anti-CEA antibodies) and pharmaceutically acceptable formations thereof can be combined with one or more NeoTCR Products. In certain embodiments, the CEA agent is an anti-CEA antibody fused to an IL2v moiety (including but limited to cergutuzumab amunaleukin). In certain embodiments, the CEA agent is a bispecific antibody wherein the bispecific antibody binds to CD3 and CEA.

CEACAM Agents

In certain embodiments, agents that target CEACAM (including but not limited to anti-CEACAM antibodies) and pharmaceutically acceptable formations thereof can be combined with one or more NeoTCR Products.

GPC3 Agents

In certain embodiments, agents that target GPC3 (including but not limited to anti-GPC3 antibodies) and pharmaceutically acceptable formations thereof can be combined with one or more NeoTCR Products.

TIM3 Agents

In certain embodiments, agents that target TIM3 (including but not limited to anti-TIM3 antibodies and bispecific anti-TIM3+anti-PD-1 antibodies) and pharmaceutically acceptable formations thereof can be combined with one or more NeoTCR Products.

PI3K Inhibitors

In certain embodiments, PI3K inhibitors and pharmaceutically acceptable formations thereof can be combined with one or more NeoTCR Products.

BET Inhibitors

In certain embodiments, BET inhibitors and pharmaceutically acceptable formations thereof can be combined with one or more NeoTCR Products.

Selective Estrogen Receptor Degraders

In certain embodiments, Selective Estrogen Receptor Degraders and pharmaceutically acceptable formations thereof can be combined with one or more NeoTCR Products.

Personalized Neoantigen Vaccines

In certain embodiments, personalized neoantigen vaccines can be combined with one or more NeoTCR Products. In certain embodiments, the vaccine is a DNA vaccine. In certain embodiments, the vaccine is an RNA vaccine.

HER2 Agents

In certain embodiments, HER2 agents and pharmaceutically acceptable formations thereof can be combined with one or more NeoTCR Products. In certain embodiments, the HER2 agent is an anti-HER2 antibody. In certain embodiments, the anti-HER2 agent is an anti-HER2+anti-CD3 bispecific antibody. In certain embodiments, the anti-HER2 antibody is trastuzumab. In certain embodiments, the anti-HER2 antibody is ado-trastuzurnab emtansine. In certain embodiments, the anti-HER2 antibody is pertuzumab.

CD20 Agents

In certain embodiments, CD20 agents and pharmaceutically acceptable formations thereof can be combined with one or more NeoTCR Products. In certain embodiments, the CD20 agent is an anti-CD20 antibody. In certain embodiments, the anti-CD20 antibody is rituxumab. In certain embodiments, the anti-CD20 antibody is obinutuzumab. In certain embodiments, the anti-CD20 antibody is an anti-CD20+anti-CD3 bispecific antibody.

MEK Inhibitors

In certain embodiments, MEK inhibitors and pharmaceutically acceptable formations thereof can be combined with one or more NeoTCR Products. In certain embodiments, the MEK inhibitor is cobimetinib.

BRAF Inhibitors

In certain embodiments, BRAF inhibitors and pharmaceutically acceptable formations thereof can be combined with one or more NeoTCR Products. In certain embodiments, the BRAF inhibitor is vemurafenib.

MEK Inhibitors and BRAF Inhibitors

In certain embodiments, BRAF inhibitors and pharmaceutically acceptable formations thereof and MEK inhibitors and pharmaceutically acceptable formations thereof can be combined with one or more NeoTCR Products. In certain embodiments, the BRAF inhibitor is vemurafenib and the MEK inhibitor is cobimetinib.

AKT Inhibitors

In certain embodiments, AKT inhibitors and pharmaceutically acceptable formations thereof can be combined with one or more NeoTCR Products. In certain embodiments, the AKT inhibitor is Ipatasertib.

ALK Inhibitors

In certain embodiments, ALK inhibitors and pharmaceutically acceptable formations thereof can be combined with one or more NeoTCR Products. In certain embodiments, the ALK inhibitor is Alectanib.

EGFR Inhibitors

In certain embodiments, EGFR inhibitors and pharmaceutically acceptable formations thereof can be combined with one or more NeoTCR Products. In certain embodiments, the EGFR inhibitor is erlotinib.

Bcl2 Inhibitors

In certain embodiments, AKT inhibitors and pharmaceutically acceptable formations thereof can be combined with one or more NeoTCR Products. In certain embodiments, the AKT inhibitor is Venetoclax.

MDM2 Inhibitors

In certain embodiments, AKT inhibitors and pharmaceutically acceptable formations thereof can be combined with one or more NeoTCR Products. In certain embodiments, the AKT inhibitor is Idasanutlin.

NTRK/ROS1 Inhibitors

In certain embodiments, NTRK/ROS1 inhibitors and pharmaceutically acceptable formations thereof can be combined with one or more NeoTCR Products. In certain embodiments, the NTRK/ROS1 inhibitor is Entrectanib.

Anti-CD40 Antibodies

In certain embodiments, anti-CD40 antibodies and pharmaceutically acceptable formations thereof can be combined with one or more NeoTCR Products.

Anti-CD79b Antibodies

In certain embodiments, anti-CD40 antibodies and pharmaceutically acceptable formations thereof can be combined with one or more NeoTCR Products.

Capecitabine

In certain embodiments, capecitabine and pharmaceutically acceptable formations thereof can be combined with one or more NeoTCR Products.

### Example 4. Solid Tumor Indications

NeoTCR Products can be used for the treatment of any cancer of which one or more NeoTCRs can be detected. In certain embodiments, NeoTCR Products can be used for the treatment of solid tumors.

In certain embodiments, one or more NeoTCR Product can be combined with one or more chemotherapeutic agent or radiation therapy for the treatment of solid tumors. In certain embodiments, one or more of the combination agents described in Example 3 can be used in combination with one or more NeoTCR Product for the treatment of solid tumors. In certain embodiments, one or more of the combination agents described in Example 3 can be used in combination with one or more NeoTCR Product and radiation for the treatment of solid tumors.

Method of Treating Breast Cancer

In certain embodiments, NeoTCR Products can be used to treat breast cancer.

In certain embodiments, NeoTCR Products can be used to treat hormone receptor (ER+ and/or PR+) positive breast cancer.

In certain embodiments, NeoTCR Products can be used to treat Her2 negative (Her2−) breast cancer.

In certain embodiments, NeoTCR Products can be used to treat hormone receptor positive (ER+ and/or PR+) breast cancer.

In certain embodiments, NeoTCR Products can be used to treat Her2 positive (Her2+) breast cancer.

In certain embodiments, NeoTCR Products can be used to treat hormone receptor negative (ER−) breast cancer.

In certain embodiments, NeoTCR Products can be used to treat hormone receptor negative (ER−) and Her2 negative (Her2−) (i.e., triple negative) breast cancer.

In certain embodiments, NeoTCR Products can be used to treat hormone receptor (ER+ and/or PR+) positive and Her2 negative (Her2−) breast cancer.

In certain embodiments, NeoTCR Products can be used to treat hormone receptor positive (ER+ and/or PR+) and Her2 positive (Her2+) breast cancer.

In certain embodiments, NeoTCR Products can be used to treat hormone receptor negative (ER−) and Her2 positive (Her2+) breast cancer.

In certain embodiments, NeoTCR Products can be used to treat BRCA positive (BRCA+) breast cancer.

In certain embodiments, one or more of the combination agents described in Example 3 can be used in combination with one or more NeoTCR Product for the treatment of breast cancer. In certain embodiments, one or more of the combination agents described in Example 3 can be used in combination with one or more NeoTCR Product and radiation for the treatment of breast cancer.

Method of Treating Thoracic Cancer

In certain embodiments, NeoTCR Products can be used to treat lung cancer.

In certain embodiments, the lung cancer is non-small cell lung cancer (NSCLC). In certain embodiments, the non-small cell lung cancer is a squamous cell carcinoma. In certain embodiments, the non-small cell lung cancer is an adenocarcinoma. In certain embodiments, the non-small cell lung cancer is a large cell carcinoma. In certain embodiments, the non-small cell lung cancer is an adenosquamous carcinoma. In certain embodiments, the non-small cell lung cancer is an undifferentiated carcinoma. In certain embodiments, the non-small cell lung cancer is a combination of more than one type of NSCLC.

In certain embodiments, one or more of the combination agents described in Example 3 can be used in combination with one or more NeoTCR Product for the treatment of NSCLC. In certain embodiments, one or more of the combination agents described in Example 3 can be used in combination with one or more NeoTCR Product and radiation for the treatment of NSCLC.

In certain embodiments, the lung cancer is small cell lung cancer (SCLC).

In certain embodiments, one or more of the combination agents described in Example 3 can be used in combination with one or more NeoTCR Product for the treatment of SCLC. In certain embodiments, one or more of the combination agents described in Example 3 can be used in combination with one or more NeoTCR Product and radiation for the treatment of SCLC.

In certain embodiments, the lung cancer is mesothelioma.

In certain embodiments, one or more of the combination agents described in Example 3 can be used in combination with one or more NeoTCR Product for the treatment of mesothelioma. In certain embodiments, one or more of the combination agents described in Example 3 can be used in combination with one or more NeoTCR Product and radiation for the treatment of mesothelioma.

Method of Treating Prostate Cancer

In certain embodiments, NeoTCR Products can be used to treat prostate cancer.

In certain embodiments, the prostate cancer is acinar adenocarcinoma. In certain embodiments, the prostate cancer is a ductal adenocarcinoma. In certain embodiments, the prostate cancer is a transitional cell (or urothelial) cancer. In certain embodiments, the prostate cancer is squamous cell cancer. In certain embodiments, the prostate cancer is a small cell prostate cancer. In certain embodiments, the prostate cancer is a neuroendocrine cancer. In certain embodiments, the prostate cancer is a sarcoma. In certain embodiments, the prostate cancer is a combination of more than one type of prostate cancer.

In certain embodiments, one or more of the combination agents described in Example 3 can be used in combination with one or more NeoTCR Product for the treatment of prostate cancer. In certain embodiments, one or more of the combination agents described in Example 3 can be used in combination with one or more NeoTCR Product and radiation for the treatment of prostate cancer.

Method of Treating Colorectal Cancer

In certain embodiments, NeoTCR Products can be used to treat colorectal cancer.

In certain embodiments, the colorectal cancer is microsatellite stable. In certain embodiments, the colorectal cancer is microsatellite instable.

In certain embodiments, the colorectal cancer is colorectal adenocarcinoma. In certain embodiments, the colorectal cancer is a gastrointestinal carcinoid tumors. In certain embodiments, the colorectal cancer is a primary colorectal lymphoma. In certain embodiments, the colorectal cancer is gastrointestinal stromal tumors. In certain embodiments, the colorectal cancer is a leiomyosrcoma. In certain embodiments, the colorectal cancer is a combination of more than one type of colorectal cancer.

In certain embodiments, one or more of the combination agents described in Example 3 can be used in combination with one or more NeoTCR Product for the treatment of colorectal cancer. In certain embodiments, one or more of the combination agents described in Example 3 can be used in combination with one or more NeoTCR Product and radiation for the treatment of colorectal cancer.

Method of Treating Gynecological Cancer

In certain embodiments, NeoTCR Products can be used to treat ovarian cancer.

In certain embodiments, the ovarian cancer is acinar epithelial ovarian cancer. In certain embodiments, the ovarian cancer is a germ cell ovarian cancer. In certain embodiments, the ovarian cancer is a stromal cell ovarian cancer. In certain embodiments, the ovarian cancer is small cell carcinoma. In certain embodiments, the ovarian cancer is a platinum sensitive ovarian cancer. In certain embodiments, the ovarian cancer is a platinum resistant cancer. In certain embodiments, the ovarian cancer is a combination of more than one type of ovarian cancer.

In certain embodiments, one or more of the combination agents described in Example 3 can be used in combination with one or more NeoTCR Product for the treatment of ovarian cancer. In certain embodiments, one or more of the combination agents described in Example 3 can be used in combination with one or more NeoTCR Product and radiation for the treatment of ovarian cancer.

In certain embodiments, NeoTCR Products can be used to treat uterine cancer.

In certain embodiments, the uterine cancer is endometrial cancer. In certain embodiments, the uterine cancer is a uterine sarcoma. In certain embodiments, the endometrial cancer is a serous adenocarcinoma. In certain embodiments, the endometrial cancer is adenosquamous carcinoma. In certain embodiments, the endometrial cancer is a uterine carcinoma. In certain embodiments, the uterine sarcoma is a uterine leiomyosarcoma. In certain embodiments, the uterine sarcoma is endometrial stromal sarcoma. In certain embodiments, the uterine sarcoma is undifferentiated sarcoma. In certain embodiments, the uterine cancer is a combination of more than one type of ovarian cancer.

In certain embodiments, one or more of the combination agents described in Example 3 can be used in combination with one or more NeoTCR Product for the treatment of uterine cancer. In certain embodiments, one or more of the combination agents described in Example 3 can be used in combination with one or more NeoTCR Product and radiation for the treatment of uterine cancer.

In certain embodiments, NeoTCR Products can be used to treat cervical cancer.

In certain embodiments, the cervical cancer is a squamous cell carcinoma. In certain embodiments, the cervical cancer is an adenocarcinoma. In certain embodiments, the cervical cancer is a combination of more than one type of cervical cancer.

In certain embodiments, one or more of the combination agents described in Example 3 can be used in combination with one or more NeoTCR Product for the treatment of uterine cancer. In certain embodiments, one or more of the combination agents described in Example 3 can be used in combination with one or more NeoTCR Product and radiation for the treatment of uterine cancer.

In certain embodiments, NeoTCR Products can be used to treat vaginal cancer.

In certain embodiments, the vaginal cancer is squamous cell carcinoma. In certain embodiments, the vaginal cancer is a adenocarcinoma. In certain embodiments, the vaginal cancer is a clear cell adenocarcinoma. In certain embodiments, the vaginal cancer is a melanoma. In certain embodiments, the vaginal cancer is a combination of more than one type of vaginal cancer.

In certain embodiments, one or more of the combination agents described in Example 3 can be used in combination with one or more NeoTCR Product for the treatment of vaginal cancer. In certain embodiments, one or more of the combination agents described in Example 3 can be used in combination with one or more NeoTCR Product and radiation for the treatment of vaginal cancer.

In certain embodiments, NeoTCR Products can be used to treat vulvar cancer.

In certain embodiments, the vulvar cancer is vulvar squamous cell carcinoma. In certain embodiments, the vulvar cancer is a melanoma. In certain embodiments, the vulvar cancer is a combination of more than one type of vulvar cancer.

In certain embodiments, one or more of the combination agents described in Example 3 can be used in combination with one or more NeoTCR Product for the treatment of vulvar cancer. In certain embodiments, one or more of the combination agents described in Example 3 can be used in combination with one or more NeoTCR Product and radiation for the treatment of vulvar cancer.

Method of Treating Pancreatic Cancer

In certain embodiments, NeoTCR Products can be used to treat pancreatic cancer.

In certain embodiments, the pancreatic cancer is exocrine tumors. In certain embodiments, the pancreatic cancer is endocrine tumors. In certain embodiments, the endocrine tumors can an insulinoma, glucagonoma, gastinoma, somatostatinoma, VIPomas, or PPomas. In certain embodiments, the pancreatic cancer is a combination of more than one type of pancreatic cancer.

In certain embodiments, one or more of the combination agents described in Example 3 can be used in combination with one or more NeoTCR Product for the treatment of pancreatic cancer. In certain embodiments, one or more of the combination agents described in Example 3 can be used in combination with one or more NeoTCR Product and radiation for the treatment of pancreatic cancer.

Method of Treating Melanoma

In certain embodiments, NeoTCR Products can be used to treat melanoma.

In certain embodiments, the melanoma is superficial spreading melanoma. In certain embodiments, the melanoma is nodular melanoma. In certain embodiments, the melanoma is lentigo maligna melanoma. In certain embodiments, the melanoma is acral lentiginous melanoma. In certain embodiments, the melanoma is desmoplastic melanoma. In certain embodiments, the melanoma is ocular melanoma. In certain embodiments, the melanoma is anorectal melanoma. In certain embodiments, the melanoma is a combination of more than one type of melanoma.

In certain embodiments, one or more of the combination agents described in Example 3 can be used in combination with one or more NeoTCR Product for the treatment of melanoma. In certain embodiments, one or more of the combination agents described in Example 3 can be used in combination with one or more NeoTCR Product and radiation for the treatment of melanoma.

Method of Treating Central Nervous System Cancers

In certain embodiments, NeoTCR Products can be used to treat central nervous system cancers (CNS cancers).

In certain embodiments, the CNS cancer is brain tumors. In certain embodiments, the CNS cancer is a CNS metastasis. In certain embodiments, the CNS cancer is CNS tumors. In certain embodiments, the CNS cancer is glioblastoma multiforme. In certain embodiments, the CNS cancer is a meningioma. In certain embodiments, the CNS cancer is skull base cancer. In certain embodiments, the CNS cancer is spinal cord tumors. In certain embodiments, the CNS cancer is a glioma. In certain embodiments, the CNS cancer is a astrocytoma. In certain embodiments, the CNS cancer is a brain stem glioma. In certain embodiments, the CNS cancer is a ependymoma. In certain embodiments, the CNS cancer is a germ cell tumor. In certain embodiments, the CNS cancer is a medulloblastoma. In certain embodiments, the CNS cancer is a combination of more than one type of CNS cancer.

In certain embodiments, one or more of the combination agents described in Example 3 can be used in combination with one or more NeoTCR Product for the treatment of CNS cancer. In certain embodiments, one or more of the combination agents described in Example 3 can be used in combination with one or more NeoTCR Product and radiation for the treatment of CNS cancer.

Method of Treating Cutaneous Cancers

In addition to the treatment of melanomas described above, in certain embodiments NeoTCR Products can be used to treat other cutaneous cancers.

In certain embodiments, the cutaneous cancer is cutaneous melanoma. In certain embodiments, the cutaneous cancer is a mucosal melanoma. In certain embodiments, the cutaneous cancer is acral melanoma. In certain embodiments, the cutaneous cancer is basal cell carcinoma. In certain embodiments, the cutaneous cancer is a Merkel cell carcinoma. In certain embodiments, the cutaneous cancer is squamous cell carcinoma. In certain embodiments, the cutaneous cancer is a combination of more than one type of cutaneous cancer.

In certain embodiments, one or more of the combination agents described in Example 3 can be used in combination with one or more NeoTCR Product for the treatment of cutaneous cancer. In certain embodiments, one or more of the combination agents described in Example 3 can be used in combination with one or more NeoTCR Product and radiation for the treatment of cutaneous cancer.

Method of Treating Head and Neck Cancers

In certain embodiments, NeoTCR Products can be used to treat Head and Neck cancers.

In certain embodiments, the Head and Neck cancer is salivary gland tumors. In certain embodiments, the Head and Neck cancer is a head and neck squamous cell carcinoma. In certain embodiments, the Head and Neck cancer is nasopharyngeal carcinoma. In certain embodiments, the Head and Neck cancer is a combination of more than one type of Head and Neck cancer.

In certain embodiments, one or more of the combination agents described in Example 3 can be used in combination with one or more NeoTCR Product for the treatment of Head and Neck cancer. In certain embodiments, one or more of the combination agents described in Example 3 can be used in combination with one or more NeoTCR Product and radiation for the treatment of Head and Neck cancer.

Method of Treating HPV+ Cancers

In certain embodiments, NeoTCR Products can be used to treat HPV+ cancers.

In certain embodiments, one or more of the combination agents described in Example 3 can be used in combination with one or more NeoTCR Product for the treatment of HPV+ cancer. In certain embodiments, one or more of the combination agents described in Example 3 can be used in combination with one or more NeoTCR Product and radiation for the treatment of HPV+ cancer.

Method of Treating Esophageal Cancers

In certain embodiments, NeoTCR Products can be used to treat esophageal cancers.

In certain embodiments, the esophageal cancer is an adenocarcinoma. In certain embodiments, the esophageal cancer is a squamous cell carcinoma. In certain embodiments, the esophageal cancer is a combination of more than one type of esophageal cancer.

In certain embodiments, one or more of the combination agents described in Example 3 can be used in combination with one or more NeoTCR Product for the treatment of esophageal cancer. In certain embodiments, one or more of the combination agents described in Example 3 can be used in combination with one or more NeoTCR Product and radiation for the treatment of esophageal cancer.

Method of Treating Thyroid Cancers

In certain embodiments, NeoTCR Products can be used to treat thyroid cancers.

In certain embodiments, the thyroid cancer is papillary cancer. In certain embodiments, the thyroid cancer is follicular. In certain embodiments, the thyroid cancer is anaplastic cancer. In certain embodiments, the thyroid cancer is medullary. In certain embodiments, the thyroid cancer is a combination of more than one type of thyroid cancer.

In certain embodiments, one or more of the combination agents described in Example 3 can be used in combination with one or more NeoTCR Product for the treatment of thyroid cancer. In certain embodiments, one or more of the combination agents described in Example 3 can be used in combination with one or more NeoTCR Product and radiation for the treatment of thyroid cancer.

Method of Treating Gastric Cancers

In certain embodiments, NeoTCR Products can be used to treat gastric cancers.

In certain embodiments, one or more of the combination agents described in Example 3 can be used in combination with one or more NeoTCR Product for the treatment of gastric cancer. In certain embodiments, one or more of the combination agents described in Example 3 can be used in combination with one or more NeoTCR Product and radiation for the treatment of gastric cancer.

Method of Treating Hepatocellular Cancers

In certain embodiments, NeoTCR Products can be used to treat hepatocellular cancers.

In certain embodiments, one or more of the combination agents described in Example 3 can be used in combination with one or more NeoTCR Product for the treatment of hepatocellular cancer. In certain embodiments, one or more of the combination agents described in Example 3 can be used in combination with one or more NeoTCR Product and radiation for the treatment of hepatocellular cancer.

Method of Treating Cholangiocarcinoma

In certain embodiments, NeoTCR Products can be used to treat cholangiocarcinoma.

In certain embodiments, one or more of the combination agents described in Example 3 can be used in combination with one or more NeoTCR Product for the treatment of cholangiocarcinoma. In certain embodiments, one or more of the combination agents described in Example 3 can be used in combination with one or more NeoTCR Product and radiation for the treatment of cholangiocarcinoma.

Method of Treating Renal Cell Cancers

In certain embodiments, NeoTCR Products can be used to treat renal cell cancers.

In certain embodiments, the renal cell cancer is clear cell renal cancer. In certain embodiments, the renal cell cancer is non-clear cell renal cancer. In certain embodiments, the renal cell cancer is a combination of more than one type of renal cell cancer.

In certain embodiments, one or more of the combination agents described in Example 3 can be used in combination with one or more NeoTCR Product for the treatment of renal cell cancer. In certain embodiments, one or more of the combination agents described in Example 3 can be used in combination with one or more NeoTCR Product and radiation for the treatment of renal cell cancer.

Method of Treating Bladder Cancers

In certain embodiments, NeoTCR Products can be used to treat bladder cancers.

In certain embodiments, the bladder cancer is urothelial carcinoma. In certain embodiments, the bladder cancer is squamous cell carcinoma. In certain embodiments, the bladder cancer is adenocarcinoma. In certain embodiments, the bladder cancer is a combination of more than one type of bladder cancer.

In certain embodiments, one or more of the combination agents described in Example 3 can be used in combination with one or more NeoTCR Product for the treatment of bladder cancers. In certain embodiments, one or more of the combination agents described in Example 3 can be used in combination with one or more NeoTCR Product and radiation for the treatment of bladder cancers.

Method of Treating Testicular Cancer

In certain embodiments, NeoTCR Products can be used to treat testicular cancer.

In certain embodiments, one or more of the combination agents described in Example 3 can be used in combination with one or more NeoTCR Product for the treatment of testicular cancer. In certain embodiments, one or more of the combination agents described in Example 3 can be used in combination with one or more NeoTCR Product and radiation for the treatment of testicular cancer.

Method of Treating Sarcomas

In certain embodiments, NeoTCR Products can be used to treat sarcomas.

In certain embodiments, one or more of the combination agents described in Example 3 can be used in combination with one or more NeoTCR Product for the treatment of sarcomas. In certain embodiments, one or more of the combination agents described in Example 3 can be used in combination with one or more NeoTCR Product and radiation for the treatment of sarcomas.

### Example 5. Liquid Tumor Indications

NeoTCR Products can be used for the treatment of any cancer of which one or more NeoTCRs can be detected. In certain embodiments, NeoTCR Products can be used for the treatment of liquid or hematological tumors.

Method of Treating Follicular Lymphoma

In certain embodiments, NeoTCR Products can be used to treat follicular lymphoma.

In certain embodiments, one or more of the combination agents described in Example 3 can be used in combination with one or more NeoTCR Product for the treatment of follicular lymphoma. In certain embodiments, one or more of the combination agents described in Example 3 can be used in combination with one or more NeoTCR Product and radiation for the treatment of follicular lymphoma.

Method of Treating Leukemia

In certain embodiments, NeoTCR Products can be used to leukemia.

In certain embodiments, the leukemia is acute lymphocytic leukemia (ALL). In certain embodiments, the leukemia is acute myeloid leukemia (AML). In certain embodiments, the leukemia is chronic lymphocytic leukemia (CLL). In certain embodiments, the leukemia is chronic myeloid leukemia (CML). In certain embodiments, the leukemia is hairy cell leukemia (HCL). In certain embodiments, the leukemia is myelodysplastic syndromes leukemia (MDS). In certain embodiments, the leukemia is a combination of more than one type of leukemia.

In certain embodiments, one or more of the combination agents described in Example 3 can be used in combination with one or more NeoTCR Product for the treatment of leukemia. In certain embodiments, one or more of the combination agents described in Example 3 can be used in combination with one or more NeoTCR Product and radiation for the treatment of leukemia.

Method of Treating Multiple Myeloma

In certain embodiments, NeoTCR Products can be used to multiple myeloma.

In certain embodiments, the multiple myeloma is smouldering indolent multiple myeloma. In certain embodiments, the multiple myeloma is hyperdiploid multiple myeloma. In certain embodiments, the multiple myeloma is non-hyperdiploid (hypodiploid) multiple myeloma. In certain embodiments, the multiple myeloma is light chain myeloma. In certain embodiments, the multiple myeloma is non-secretory myeloma. In certain embodiments, the multiple myeloma is solitary plasmacytoma. In certain embodiments, the multiple myeloma is extramedullary plasmacytoma. In certain embodiments, the multiple myeloma is monoclonal gammopathy of undetermined significance. In certain embodiments, the multiple myeloma is a combination of more than one type of multiple myeloma.

In certain embodiments, one or more of the combination agents described in Example 3 can be used in combination with one or more NeoTCR Product for the treatment of leukemia. In certain embodiments, one or more of the combination agents described in Example 3 can be used in combination with one or more NeoTCR Product and radiation for the treatment of leukemia.

### Example 6. Method of Administering the NeoTCR Product

In certain embodiments, the NeoTCR Product can be administered to a patient intravenously. In certain embodiments, the NeoTCR Product can be administered to a patient intraarterially, intraperitoneally, intracranially, intraarticularly, intrapleural, intravitreally, or intravascularly. In certain embodiments, the NeoTCR Product can be administered to a patient by injection, by infusion, by continuous infusion, by localized perfusion bathing target cells directly, by catheter, or by lavage.

Because the NeoTCR Product comprises ‘young’ cell phenotypes, the NeoTCR cells, when administered to a patient in need thereof should remain as memory T cells in the patient and should be able to proliferate in response to a cognate neoantigen. Accordingly, in certain embodiments, the NeoTCR Product is only administered one (1) time to a patient. In certain embodiments, the NeoTCR Product can be administered two (2) or more times to a patient if needed.

In certain embodiments, the number of NeoTCR cells in the NeoTCR Product that is administered to a patient in need thereof is approximately 100,000 NeoTCR cells. In certain embodiments, the number of NeoTCR cells in the NeoTCR Product that is administered to a patient in need thereof is between 100,000 NeoTCR cells and 500,000 NeoTCR cells. In certain embodiments, the number of NeoTCR cells in the NeoTCR Product that is administered to a patient in need thereof is between 500,000 NeoTCR cells and 1,000,000 NeoTCR cells. In certain embodiments, the number of NeoTCR cells in the NeoTCR Product that is administered to a patient in need thereof is between 1,00,000 NeoTCR cells and 2,000,000 NeoTCR cells. In certain embodiments, the number of NeoTCR cells in the NeoTCR Product that is administered to a patient in need thereof is between 2,00,000 NeoTCR cells and 5,000,000 NeoTCR cells. In certain embodiments, the number of NeoTCR cells in the NeoTCR Product that is administered to a patient in need thereof is between 5,00,000 NeoTCR cells and 10,000,000 NeoTCR cells. In certain embodiments, the number of NeoTCR cells in the NeoTCR Product that is administered to a patient in need thereof is more than 10,000,000 NeoTCR cells. In certain embodiments, the number of NeoTCR cells in the NeoTCR Product that is administered to a patient in need thereof is between 50,000,000 and 150,000,000 NeoTCR cells. In certain embodiments, the number of NeoTCR cells in the NeoTCR Product that is administered to a patient in need thereof is between 150,000,000 and 300,000,000 NeoTCR cells. In certain embodiments, the number of NeoTCR cells in the NeoTCR Product that is administered to a patient in need thereof is between 300,000,000 and 500,000,000 NeoTCR cells.

In certain embodiments, the number of NeoTCR cells in the NeoTCR Product that is administered to a patient can be a flat dose of 10,000,000 cells for a patient. In certain embodiments, the number of NeoTCR cells in the NeoTCR Product that is administered to a patient can be a flat dose of 1,000,000 cells for a patient. In certain embodiments, the approximate weight of a patient for such fixed doses is 50 kg.

In certain embodiments, the number of NeoTCR cells in the NeoTCR Product that is administered to a patient can be a flat dose of 400,000,000 cells for a patient. In certain embodiments, the approximate weight of a patient for such fixed doses is 50 kg.

In certain embodiments, the number of NeoTCR cells in the NeoTCR Product that is administered to a patient can be dosed at 1,000,000 cells per kg. In certain embodiments, the number of NeoTCR cells in the NeoTCR Product that is administered to a patient can be dosed at 2,000,000 cells per kg. In certain embodiments, the number of NeoTCR cells in the NeoTCR Product that is administered to a patient can be dosed at 3,000,000 cells per kg. In certain embodiments, the number of NeoTCR cells in the NeoTCR Product that is administered to a patient can be dosed at 4,000,000 cells per kg. In certain embodiments, the number of NeoTCR cells in the NeoTCR Product that is administered to a patient can be dosed at 5,000,000 cells per kg. In certain embodiments, the number of NeoTCR cells in the NeoTCR Product that is administered to a patient can be dosed at 6,000,000 cells per kg. In certain embodiments, the number of NeoTCR cells in the NeoTCR Product that is administered to a patient can be dosed at 7,000,000 cells per kg. In certain embodiments, the number of NeoTCR cells in the NeoTCR Product that is administered to a patient can be dosed at 8,000,000 cells per kg. In certain embodiments, the number of NeoTCR cells in the NeoTCR Product that is administered to a patient can be dosed at 9,000,000 cells per kg. In certain embodiments, the number of NeoTCR cells in the NeoTCR Product that is administered to a patient can be dosed at 10,000,000 cells per kg. In certain embodiments, the number of NeoTCR cells in the NeoTCR Product that is administered to a patient can be dosed at more than 10,000,000 cells per kg. In certain embodiments, the number of NeoTCR cells in the NeoTCR Product that is administered to a patient can be dosed at less than 1,000,000 cells per kg.

In certain embodiments, any of the total number of cells administered to a patient described herein can be administered as a single dose, split between two doses (i.e., administered at two different time points), or split between three or more doses (i.e., administered at three or more different time points).

### Example 7. Safety Pharmacology of the NeoTCR Product

Using the NeoTCR isolation technology neoE-specific T cells are captured from the subject's peripheral blood by their ability to specifically bind to the neoepitope peptide derived from the subject's tumor-exclusive mutated protein (neoantigen) and complexed with one of that subject's HLA receptors. Furthermore, only NeoTCRs derived from antigen-experienced T cells are selected for NeoTCR Product development, based on the premise that the candidate neoE-specific T cells had become activated in response to encounter with the cognate neoE peptide-HLA. The tumor exclusive presentation of the neoE-HLA target had resulted in neoE-specific T cell activation and expansion yet did not trigger auto-immune responses in the patient. These elements of personalization de-risk the potential safety/tolerability profile following infusion of the adoptively transferred NeoTCR cells.

Two non-native proteins are involved in the precision genome engineering process: Cas9 ribonucleoprotein, which is delivered transiently and subsequently degraded in dividing cells; and P2A peptides introduced as part of the NeoTCR gene cassette. Although Cas9 is a non-native protein that is transiently delivered during the engineering process, there is no evidence of residual Cas9 protein in the final product, thus de-risking the potential for anti-Cas9 immunogenicity following administration of the NeoTCR Product. While the P2A sequences are the sole non-human sequence in the NeoTCR gene cassette, they are not expressed as part of the mature NeoTCR polypeptides on the engineered T cell surface, and therefore, do not represent an opportunity for antibody-dependent T cell-mediated cytotoxicity.

Comprehensive in vitro and in silico analyses have been conducted to survey potential off-target sites of CRISPR/Cas9-mediated genome editing, using COSMID and GUIDE-seq, respectively. To date, multiple laboratory-scale or clinical-scale manufactured NeoTCR Products have been assessed for cleavage of the candidate genomic off-target sites by deep sequencing, affirming the published evidence that the selected CRISPR/Cas9 ribonucleoproteins used in the precision genome engineering manufacturing process are highly specific.

Additional aspects of the precision genome engineering process, such as genomic instability, have been assessed for safety. No evidence of genomic instability was found in assessing multiple NeoTCR Products by either targeted locus amplification (TLA) or standard fluorescence in situ hybridization (FISH) cytogenetics.

Taken together, the analysis of CRISPR/Cas9 ribonucleoprotein engineering by the precision genome engineering process shows exquisite specificity for on-target editing. Thus, the autologous NeoTCR Products manufactured by the precision genome engineering process should be well tolerated following re-infusion into the patients.

### Example 8. Pharmacokinetics and Product Metabolism

NeoTCR Products comprise live autologous patient T cells. The in vivo biodistribution and cell fate of the transferred cells is dependent on multiple parameters, such as the fitness and cell phenotype upon infusion, antigen-encounter after infusion, and signals received in the tumor microenvironment. Therefore, the NeoTCR cells in the NeoTCR Product should remain via memory and proliferation in the patients to whom it is administered for an extended period of time.

In certain embodiments, the NeoTCR cells administered to a patient will persist in the patient for less than one (1) year. In certain embodiments, the NeoTCR cells administered to a patient will persist in the patient between one (1) year and less than two (2) years. In certain embodiments, the NeoTCR cells administered to a patient will persist in the patient for an interval between two (2) year and less than five (5) years. In certain embodiments, the NeoTCR cells administered to a patient will persist in the patient for an interval between five (5) year and less than seven (7) years. In certain embodiments, the NeoTCR cells administered to a patient will persist in the patient for an interval between seven (7) year and less than ten (10) years. In certain embodiments, the NeoTCR cells administered to a patient will persist in the patient for an interval between ten (10) year and less than fifteen (15) years. In certain embodiments, the NeoTCR cells administered to a patient will persist in the patient for an interval between fifteen (15) year and less than twenty (20) years. In certain embodiments, the NeoTCR cells administered to a patient will persist in the patient for the lifespan of the patient.

### Example 9. Tumorigenicity

NeoTCR Products comprise precision genome engineered T cells wherein one or more NeoTCR is integrated into the genome of the T cell at a natural TCR genomic site without any artificial promoters. As a result, the NeoTCRs are expressed in the T cells to without artificial promoters.

Artificial sequences (e.g., promoters that are not endogenously present in a specific T cell) are known to cause tumorigenicity. Accordingly, because NeoTCR Products do not contain artificial promoters the potential for tumorigenicity is de-risked.

In certain embodiments, there is no tumorigenicity associated with NeoTCR Products.

### Example 10. Preconditioning Regimen

The NeoTCR Product can be administered, for example, by intravenous infusion as a single dose or in combination with one or more pharmaceutical agents. The NeoTCR Product can be administered to patients after a non-myeloablative (NMA) conditioning regimen or absent a NMA conditioning regimen.

### Example 11. Methods Reducing the Risk of and/or Treating Febrile Reaction or Febrile Neutropenia in Patients Treated with a NeoTCR Product

In certain embodiments, in order to prevent febrile reactions to a NeoTCR patients can be administered acetaminophen and diphenhydramine prior to the administration of a NeoTCR Product. In certain embodiments, the dose of such acetaminophen and diphenhydramine should be based on standard of care for each respective drug.

In certain embodiments, if a patient who is administered a NeoTCR Product develops a fever, said patient can be further administered one or more doses of acetaminophen based on standard of care dosing of acetaminophen based on a patient's age, weight, and any other relevant factors. In certain embodiments, additional standard of care supportive measures can be administered in addition to acetaminophen if a patient who was administered a NeoTCR Product develops a fever.

In certain embodiments, a patient who is administered a NeoTCR Product should not be administered corticosteroids or nonsteroidal anti-inflammatory drugs (NSAIDs). Accordingly, in one embodiment, a patient can be administered a NeoTCR Product wherein the patient is not administered a corticosteroid or an NSAID.

In certain embodiments, patients who are administered a NeoTCR Product are observed for signs of septic infusion reactions and bacterial infection. If a patient who was administered a NeoTCR Product develops a fever greater than 1° C. rise in temperature during or immediately after such administration, the patient can be monitored for septic infusion reactions and bacterial infection. In certain embodiments, if septic infusion reactions or bacterial infection is suspected, the patient can be evaluated for the source of infection based on standard of care methods.

In certain embodiments, patients who are administered a NeoTCR Product and who are neutropenic and febrile can be administered broad-spectrum antibiotics and optionally intravenous fluids based on standard of care.

### Example 12. Methods Reducing the Risk of and/or Treating Acute Infusion Reaction and Hypersensitivity Reaction in Patients Treated with a NeoTCR Product

In certain embodiments, infusion reactions may be a possible reaction to the administration of NeoTCR Product and may manifest as acute infusion reactions with transient fever, chills, rigors, and nausea to more severe hypersensitivity reactions. In addition, there may be overlap with hypersensitivity reaction.

The NeoTCR Product is cryopreserved in DMSO. DMSO toxicity is a common complication of cryopreserved product administration. Side effects related to DMSO toxicity are generally associated with histamine release. Signs and symptoms include coughing, flushing, rash, chest tightness and wheezing, nausea and vomiting, and in more extreme cases cardiovascular instability.

Acetaminophen/paracetamol and diphenhydramine/H1 antihistamine can be administered prior to the infusion and can be repeated every 6 hours as needed. If patients develop an infusion reaction the rate of infusion can be slowed or halted. Corticosteroids should only be given to those patients already on physiologic replacement therapy, or in the case of a life-threatening emergency since this can have an adverse effect on the NeoTCR cells.

### Example 13. Methods Reducing the Risk of and/or Treating Neurotoxicity in Patients Treated with a NeoTCR Product

Neurotoxicity has been observed in patients receiving CAR-T cell therapies and can range from headache, confusion, altered level of consciousness, word-finding difficulties, dysarthria, encephalopathy, and rarely to seizure. The timing of neurological changes can be concurrent with cytokine release syndrome (CRS), following resolution, or it can develop alone without symptoms of CRS in some patients.

If signs and symptoms of CRS develop with the administration of a NeoTCR Product to a patient, the patient can be administered anti-IL6 targeted therapy or steroids according to guidelines.

### Example 14. Methods Reducing the Risk of and/or Treating Tumor Lysis Syndrome in Patients Treated with a NeoTCR Product

Tumor lysis syndrome (TLS) is relatively rare in most solid tumors and therefore patients with solid tumors are at low risk.

TLS is caused by massive tumor cell lysis with the release of large amounts of potassium, phosphate, and nucleic acids into the systemic circulation and has been described in patients with hematologic malignancies treated with CAR-T cell therapies.

Laboratory TLS is defined as 2 or more of the following values within the days following NeoTCR Product infusion: 1) Uric acid ≥8 mg/dL or 25% increase from baseline, 2) Potassium ≥6 mEq/L or 25% increase from baseline, 3) Phosphorous ≥4.5 mg/dL or 25% increase from baseline, and 4) Calcium ≤7 mg/dL or 25% decrease from baseline.

Prophylaxis can be given to patients with bulky solid tumor who have renal dysfunction and/or renal involvement or uric acid, potassium, or phosphate levels above the upper limit of normal (ULN). Patients without contraindications can be started on prophylaxis (e.g., allopurinol) as per institutional guidelines prior to NeoTCR Product infusion. Prophylaxis should be discontinued when the risk of tumor lysis has passed.

If zero or one of the laboratory values above is abnormal, the patient should continue to be administered allopurinol or a non-allopurinol alternative and hydration. In addition to allopurinol or a non-allopurinol alternative, IV hydration can be administered. Furthermore, if uric acid levels remain elevated a patient can be administered rasburicase.

### Example 15. Methods Reducing the Risk of and/or Graft Versus Host Disease and Autoimmunity in Patients Treated with a NeoTCR Product

Graft versus host disease like toxicity is a potential toxicity of NeoTCR Product that might result from the very low number of engineered NeoTCR-T cells that can harbor dual TCR species, namely one of the endogenous TCR chains in addition to expression of the NeoTCRα and β chain polypeptides. Heterologous pairing of polypeptide chains from the endogenous TCR and the NeoTCR can result in unexpected cell targeting capabilities. The potential for these events is expected to be very rare but their impact would be unpredictable since these T cells were not subjected to the thymic selection process, with the potential to trigger auto-reactivity to healthy cells.

If evidence of autoimmunity develops, patients can be treated with immune-suppressive therapies as clinically indicated based on the severity of symptoms, using medications like corticosteroids, cyclosporine-A, mycophenolate, mofetil, anti-TNFα antibodies, or anti-thymocyte globulin.

### Example 16. A NeoTCR Product Comprising Wild Type T Cells

In certain embodiments, a NeoTCR Product comprises a combination of NeoTCR cells and Wild Type (WT) T cells that have not be precision engineered to express one or more NeoTCR(s). In such a NeoTCR Product, the mixture of precision engineered NeoTCR cells in combination with WT T cells can result in epitope spreading. In certain embodiments, such a combination of NeoTCR cells and WT T cells can result in increased tumor killing and efficacy of the NeoTCR Product. In certain embodiments, the NeoTCR Product comprises at least 5% NeoTCR Cells. In certain embodiments, the NeoTCR Product comprises between approximately 5% and 10% NeoTCR Cells. In certain embodiments, the NeoTCR Product comprises between approximately 10% and 15% NeoTCR Cells. In certain embodiments, the NeoTCR Product comprises between approximately 15% and 20% NeoTCR Cells. In certain embodiments, the NeoTCR Product comprises between approximately 20% and 25% NeoTCR Cells. In certain embodiments, the NeoTCR Product comprises between approximately 25% and 30% NeoTCR Cells. In certain embodiments, the NeoTCR Product comprises between approximately 30% and 40% NeoTCR Cells. In certain embodiments, the NeoTCR Product comprises between approximately 40% and 50% NeoTCR Cells. In certain embodiments, the NeoTCR Product comprises greater than 50% NeoTCR Cells.

### Example 17. A NeoTCR Product in Combination with Genetic Circuits

In certain embodiments, a NeoTCR Product can be combined with gene circuits. The gene circuits can be used, for example, to provide targeted and localized expression of one or more combination agent or chemotherapeutic agent described herein. In certain embodiments, the gene circuits can be used, for example, to provide targeted and localized expression of one or more cytokine (or modification or derivative thereof) described herein. The gene circuits can be used, for example, to provide targeted and localized expression of one or more peptide or protein described herein. The gene circuits can be used, for example, to provide targeted and localized expression of one or more antibody described herein. In certain embodiments, the gene circuits can be used, for example, to provide controlled expression of one or more combination agent or chemotherapeutic agent described herein. In certain embodiments, the gene circuits can be used, for example, to provide controlled expression of one or more cytokine (or modification or derivative thereof) described herein. In certain embodiments, the gene circuits can be used, for example, to provide controlled expression of one or more peptide or protein described herein. The gene circuits can be used, for example, to provide controlled expression of one or more antibody described herein. In certain embodiments, the gene circuits can be used, for example, to provide multiple mechanisms of action in a cell.

### Example 18. NeoTCR Product Summary

The pharmacological evaluation of the NeoTCR Product demonstrated that the NeoTCR Product produced using an ex vivo manufacturing process without artificial promoters has potent antigen-specific killing, effector cytokine secretion, and proliferative activity on contact with cognate neoantigen-expressing tumor cells. Furthermore, the NeoTCR Product has been shown to respond to target tumor cells with a strong polyfunctional effector protein secretion response, as demonstrated by bulk T cell and single-cell secretome analysis. The observed polyfunctional T cell effector phenotype is predicted to contribute to the potential for clinical benefit upon infusion of the NeoTCR Product into patients with cancer, in a manner similar to that observed with engineered, polyfunctional, autologous CAR-T cells re-infused into patients with hematologic malignancies.

The NeoTCR Product comprises T memory stem cell (T_{MSC}) and central memory (T_{CM}) phenotypes that represent significant T cell phenotypes from achieved using the ex vivo manufacturing process without artificial promoters. T_{MSC} and T_{CM} are considered ‘younger’ cells. These ‘younger’ or less-differentiated T cell phenotypes confer improved engraftment potential and prolonged persistence post-infusion compared to engineered CAR-T cells in patients. Thus, the administration of the NeoTCR Product, comprising ‘younger’ T cell phenotypes, can benefit patients with cancer, through improved engraftment potential, prolonged persistence post-infusion, and rapid differentiation into effector T cells to eradicate tumor cells throughout the body.

Ex vivo mechanism-of-action studies were also performed with the NeoTCR Product produced with T cells from patients with cancer. Comparable gene editing efficiencies and functional activities, as measured by antigen-specificity of T cell killing activity, proliferation, and cytokine production, were observed demonstrating that the ex vivo manufacturing process without artificial promoters is successful in generating product with T cells from patients with cancer as starting material.

It was critical to interrogate the genome cleavage specificity profile of the Cas9 ribonucleoprotein nucleases used in the ex vivo manufacturing process (without artificial promoters). The NeoTCR Product manufacturing process without artificial promoters involves electroporation of dual ribonucleoprotein species of CRISPR-Cas9 nucleases bound to guide RNA sequences, each targeting the genomic TCRα and the genomic TCRβ loci. The specificity of targeting Cas9 nucleases to each genomic locus has been previously described in the literature as being highly specific. Comprehensive in vitro and in silico analyses were conducted using the NeoTCR Product to survey possible off-target genomic cleavage sites, using COSMID and GUIDE-seq, respectively. Multiple NeoTCR Products and comparable cell products from healthy donors were assessed for cleavage of the candidate off-target sites by deep sequencing, supporting the published evidence that the selected nucleases are highly specific. The results showed very low potential for Cas9 off-target activity which is not anticipated to compromise the safety, tolerability, or efficacy of the NeoTCR Product administered to patients with cancer.

Further aspects of the precision genome engineering process have been assessed for safety. No evidence of genomic instability following precision genome engineering was found in assessing multiple NeoTCR Products by targeted locus amplification (TLA) or standard FISH cytogenetics. No off-target integration anywhere into the genome of the NeoTCR sequences were detected. No evidence of residual Cas9 was found in the cell product.

Accordingly, in certain embodiments, the NeoTCR Product comprises T cells engineered to express one or more NeoTCRs that are genomically stable. In certain embodiments, the NeoTCR Product comprises T cells engineered to express one or more NeoTCRs with no off-target integration into the genome of the NeoTCR sequences. In certain embodiments, the NeoTCR Product comprises no residual Cas9. In certain embodiments, the NeoTCR Product comprises T cells engineered to express one or more NeoTCRs with no off-target integration into the genome of the NeoTCR sequences and no residual Cas9. In certain embodiments, the NeoTCR Product comprises T cells engineered to express one or more NeoTCRs with no off-target integration into the genome of the NeoTCR sequences and no residual Cas9, and wherein the NeoTCR cells in the NeoTCR Product is genomically stable.

Using any of the methods and/or compositions described herein, the administration of NeoTCR Product can eradicate tumor cells in patients for which the NeoTCR Product was designed and made and thus to provide meaningful clinical benefit for patients with cancer. Furthermore, unlike currently available cell therapies, the NeoTCR Product can eradicate tumor cells in patients for which the NeoTCR Product was designed and made and thus to provide meaningful clinical benefit for patients with solid tumors.

### Example 19. Synergistic Effect of Two or More NeoTCRs in a NeoTCR Product

The present example illustrates the efficiency of using a NeoTCR Products for the treatment of cancer.

As described herein, NeoTCR Products are designed by identifying a patient's NeoTCRs and engineering the same patient's own T cells to express the NeoTCRs in order to treat cancer. FIGS. 4A and 4B show the imPACT Isolation Technology analysis performed as described in PCT/US2020/17887 which is hereby incorporated in its entirety. The imPACT Isolation Technology was used to identify NeoTCRs from the melanoma tumor cells collected from a patient as described in FIG. 3. As shown in FIGS. 4A and 4B, multiple NeoTCRs were identified that are specific to different neoepitopes on the same gene. Specifically, 9 distinct NeoTCRs were identified that were specific to the NAT10 gene, 2 distinct NeoTCRs were identified that were specific to the PRPSAP2 gene, 4 distinct NeoTCRs were identified that were specific to the ATP11A gene, and 5 distinct NeoTCRs were identified that were specific to the HP1BP3 gene (FIG. 4A). There was also one NeoTCR identified that was specific to the UVSSA gene (FIG. 4A).

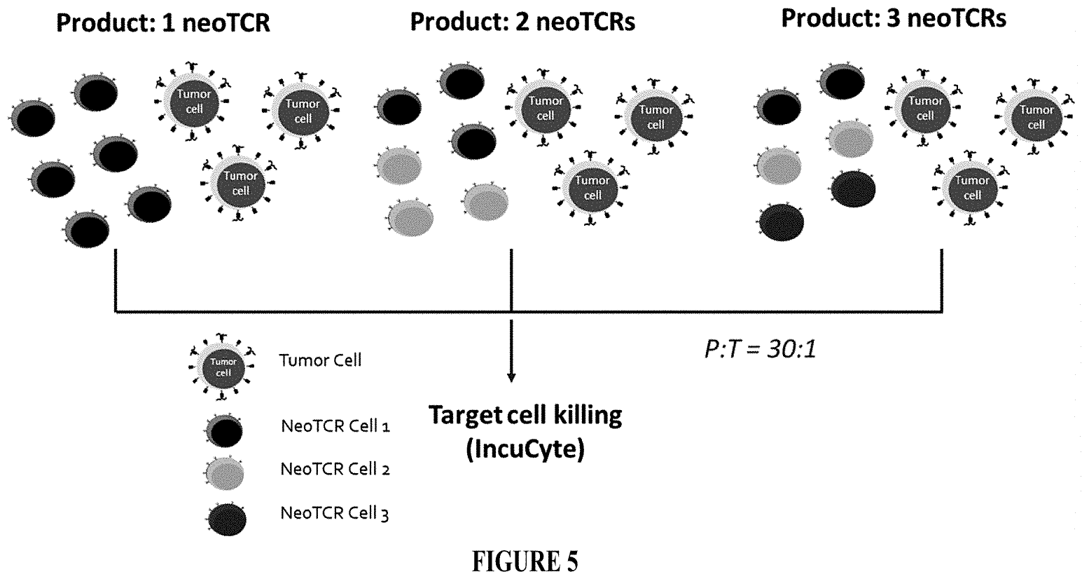

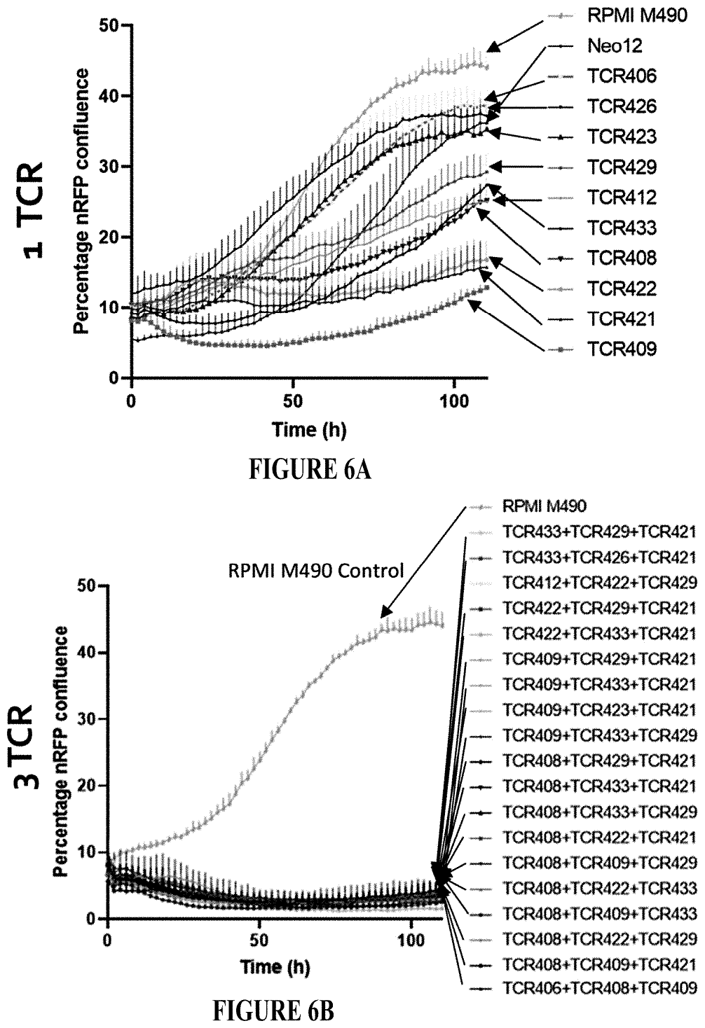

Based on the identification of different NeoTCRs, NeoTCR Products were made as described in FIG. 5. As shown, NeoTCR Cells were cocultured with autologous tumor cell line at product to target ratio (P:T) of 10:1. The NeoTCR Cells were tested as single NeoTCR Product or as a 3 NeoTCR Product composed ⅓ of each one of the 3 NeoTCRs. The NeoTCRs tested in combination targeted different neoepitopes and/or HLAs (see e.g., FIGS. 6A, 6B, 7A-7D, 8A-8E, 11A, and 11B). The autologous tumor cells were transduced to express a red fluorescent protein and the amount of red confluency was then measured over time in the IncuCyte system. The decrease in the percentage of red fluorescent protein positive cells was used as a measure of the cytotoxic activity of the NeoTCR Products. Negative controls for this experiment were: (1) tumor cells with medium alone; (2) NeoTCR Cells cocultured with tumor cell line from a different patient (i.e., NeoTCR Cells cultured with cells expressing a non-cognate antigen); (3) coculture of unrelated NeoTCR (neo12) with the autologous tumor cell line (i.e., NeoTCR Cells cultured with cells expressing a non-cognate antigen). The purpose of this experiment was to determine if there was a synergistic effect of having a NeoTCR Product comprising more than one population of NeoTCR Cells wherein the different populations express different NeoTCRs to the same gene, express different NeoTCRs to different genes, and NeoTCRs that are specific to different HLAs.

First, the killing ability of different NeoTCR Product that comprise a single population of NeoTCR Cells (i.e., the NeoTCR Product only comprises a single NeoTCR) was tested. As shown in FIG. 6A, the NeoTCR Products that comprise a single population of NeoTCR Cells have varying abilities to control the growth of the tumor cells. However, it was a surprise to find that NeoTCR Products that comprise three populations of NeoTCR Cells (i.e., the NeoTCR Product comprise three distinct populations of NeoTCR Cells, each population expressing a different NeoTCR) not only have an enhanced ability to control the growth of tumor cells but that the three different NeoTCR Cell populations in fact work together to elicit a synergistic effect to control the growth of the tumor cells (FIG. 6B; the only cell growth seen in this figure is the growth of the tumor cells in the presence of media only without a NeoTCR Product).

In order to prove that the combination of three populations of NeoTCR Cells (i.e., the NeoTCR Product comprise three distinct populations of NeoTCR Cells, each population expressing a different NeoTCR) results in greater efficacy than two populations of NeoTCR Cells, experiments were performed on every combination of NeoTCRs 408, 409, and 429 (FIGS. 7A-7D). FIG. 7A shows the comparison of each single NeoTCR Product compared to the three NeoTCR Product. FIGS. 7B-7D show the single NeoTCR Products compared to two NeoTCR Products. As shown, while the two NeoTCR Products were able to control tumor cell growth, the control was limited and some cell growth was able to occur over time. In contrast, the three NeoTCR Product shown in FIG. 7A demonstrated significantly more control of tumor growth.

The same experiment as described in FIGS. 7A-7D were performed for each of the three NeoTCR Products shown in FIG. 6B. Another example of this experiment is shown in FIGS. 8A-8D with NeoTCRs 409, 429, and 421.

Furthermore, another reason for testing each of the three NeoTCR Products shown in FIG. 6B was to explore the relevance of the specificity of each NeoTCR in the three NeoTCR Products. The combination of NeoTCRs 408, 409, and 429 (FIGS. 7A-7D) consisted of two NeoTCRs specific to the NAT10 gene and HLA-A01:01 and one NeoTCR specific to the UVSSA gene and HLA-B57:01 (FIG. 8E). The combinations of NeoTCRs 409, 429, and 421 (FIGS. 8A-8D) consisted of one NeoTCR specific to the NAT10 gene and HLA-A01:01, one NeoTCR specific to the UVSSA gene and HLA-B57:01, and one NeoTCR specific to the PRPSAP2 gene and HLA-C03:04 (FIG. 8E). As demonstrated, the ability of the NeoTCR Products to control tumor cell grows was not dependent on the variety of genes and HLAs. Rather, it was shown that it was the variety of neoepitopes that created the synergistic effect of controlling tumor cell growth with three NeoTCR Products.

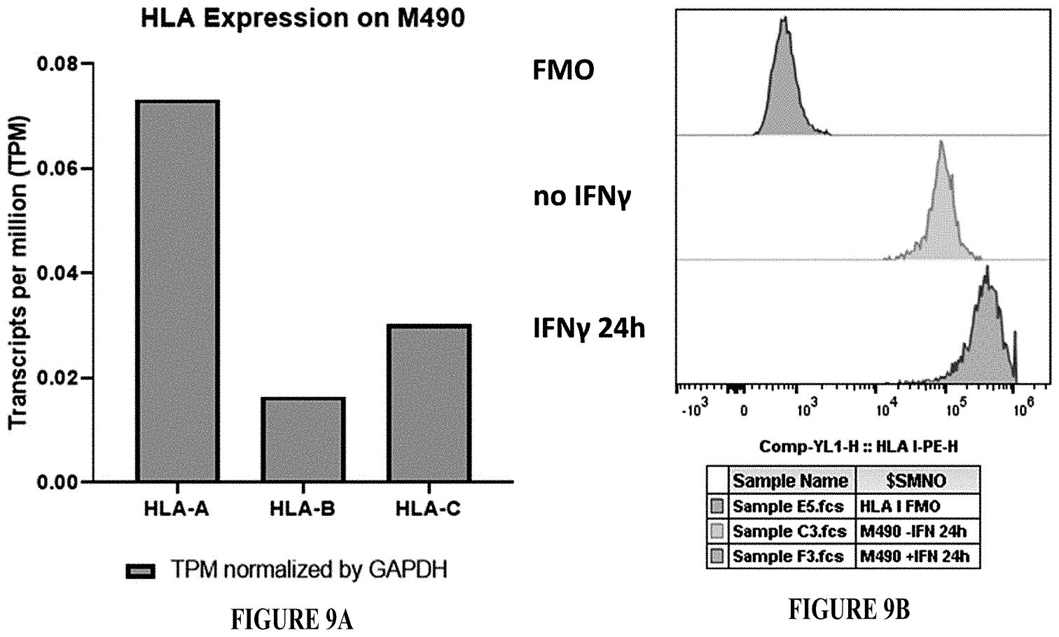

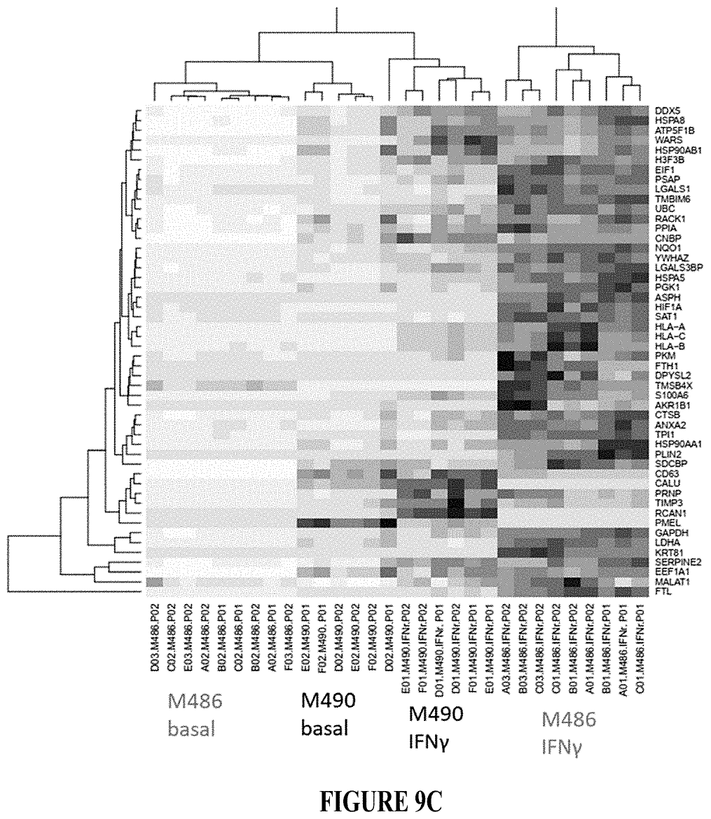

In order to rule out HLA expression level as the sole reason for the ability of NeoTCR Products to control the growth of tumor cells, HLA expression was measured in M490 tumor cells (the tumor cell line from which the NeoTCRs were identified and isolated) (FIG. 9A). Because there were different levels of expression between the classes of HLAs in the M490 tumor cells, the cells were pretreated with IFNγ for 24 hours to determine if it would be possible to increase the expression of the HLA classes (FIG. 9B). As shown in FIG. 9C, pretreatment with IFNγ resulted not only in an upregulation of HLAs but it also resulted in an upregulation of neoantigen presentation on the M490 cells. To confirm that the increased expression was not limited to the M490 cells, M486 cells were also used and a similar upregulation was observed (FIG. 9C).

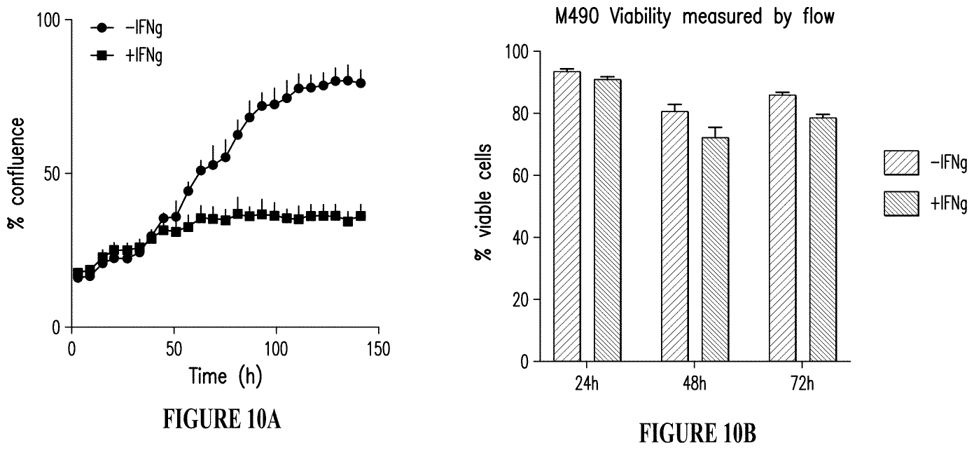

It was observed that the IFNγ appeared to slow the growth of the M490 cells. In order to better understand if the IFNγ was killing the cells or simply having a cytostatic effect, cell confluence was measured following pretreatment with IFNγ (FIG. 10A) and cell viability was concurrently measured (FIG. 10B). As demonstrated, the IFNγ pretreatement only had a cytostatic effect on the M490 cells and it did not affect cell viability.

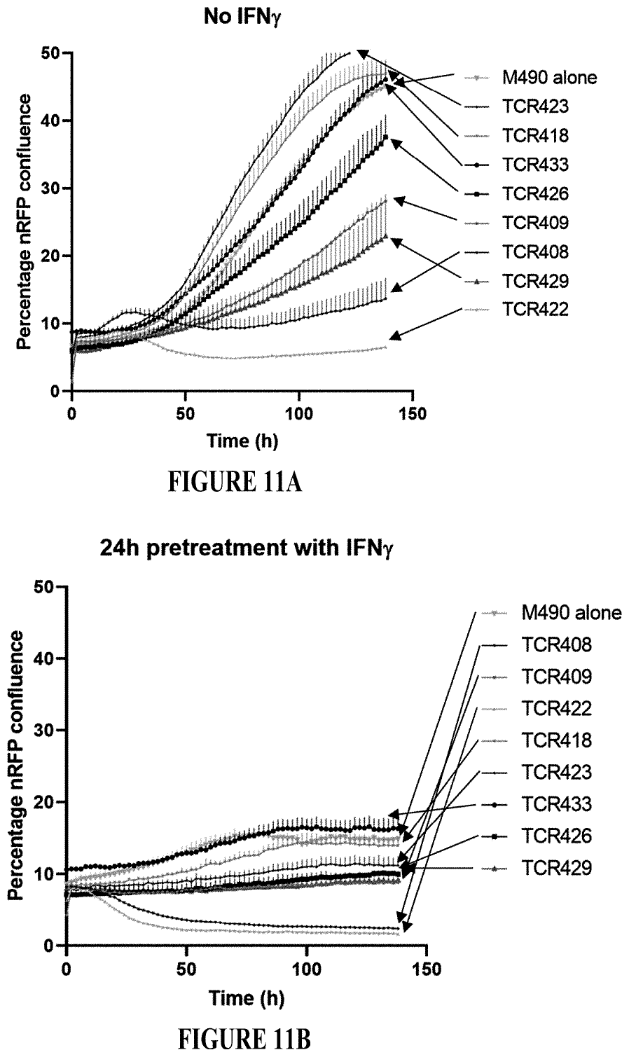

Because it was demonstrated that IFNγ pretreatment increased HLA and neoantigen presentation without affecting cell viability, experiments were designed to determine if upregulating HLA and neoantigen expression on the M490 cells could increase the ability of the NeoTCR Products to control tumor cell growth. As shown in FIGS. 11A and 11B, increasing HLA and neoantigen expression on the tumor cells was able to increase the ability of the one NeoTCR Products' control of tumor cell growth. This data lead to the question of how two and three NeoTCR Products are able to better control tumor cell growth without the addition of IFNγ.

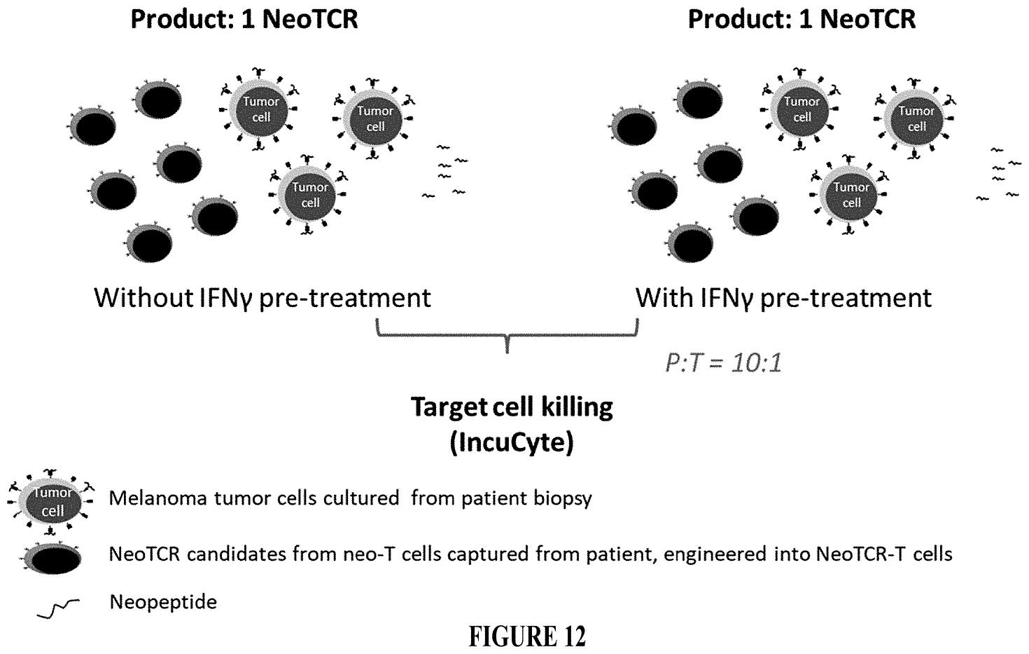

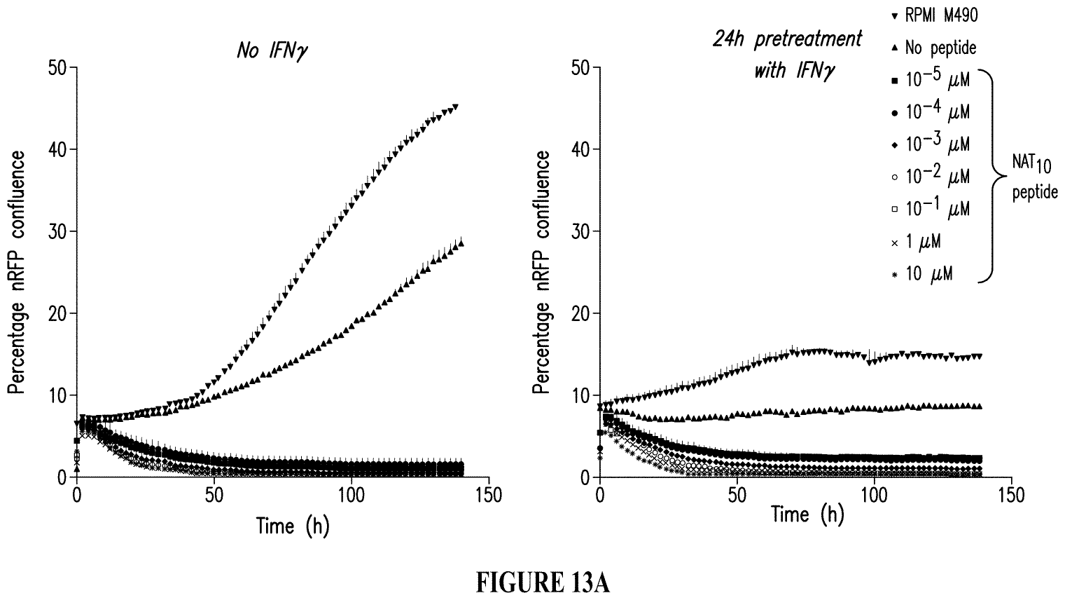

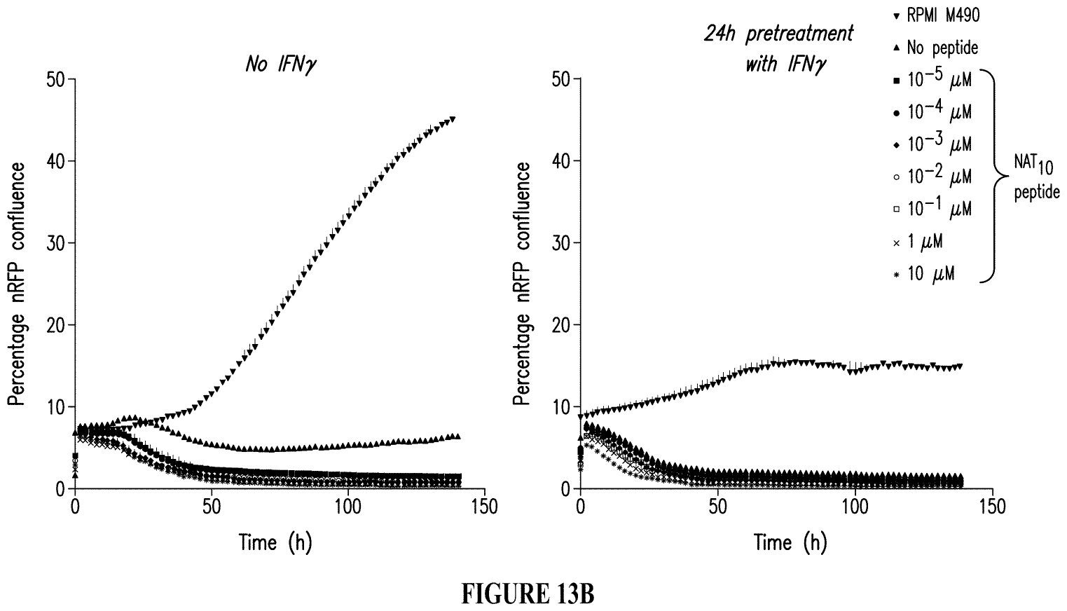

In order to explore this question, the IFNγ pretreatment results were confirmed by comparing those results with peptide pulsing experiments. The peptide pulsing experiments are described in FIG. 12. As shown, NeoTCR products expressing a single NeoTCR were pulsed with a neopeptide to increase the amount of neoantigen presented by the tumor cells in the presence or absence of IFNγ pretreatment. The results of these experiments are shown for NeoTCR 409 (FIG. 13A) and NeoTCR 422 (FIG. 13B), both of which are to the NAT10 gene. With varying amounts of NAT10 peptide pulsed into the NeoTCR Cells to increase the NAT10 neoepitope expression, the single NeoTCR Products were able to increase their ability to control tumor cell growth with and without IFNγ pretreatment. A description of the NeoTCRs is provided in Table 7 below.

TABLE 7
NeoTCR 409 and NeoTCR 422

| | | | | Dex binding | | %CD4- |
|---|---|---|---|---|---|---|
| NeoTCR | Neoepitope | Gene | HLA | to CD4 cells | %Dex+2A+ | CD8- |
| 409 | NILPISFHV | NAT10 | HLA- | No | 23.9 | 3.88 |
| | (SEQ ID NO: 1) | | A02:01 | | | |
| 422 | NILPISFHV | NAT10 | HLA- | Yes | 46.3 | 2.04 |
| | (SEQ ID NO: 1) | | A02:01 | | | |

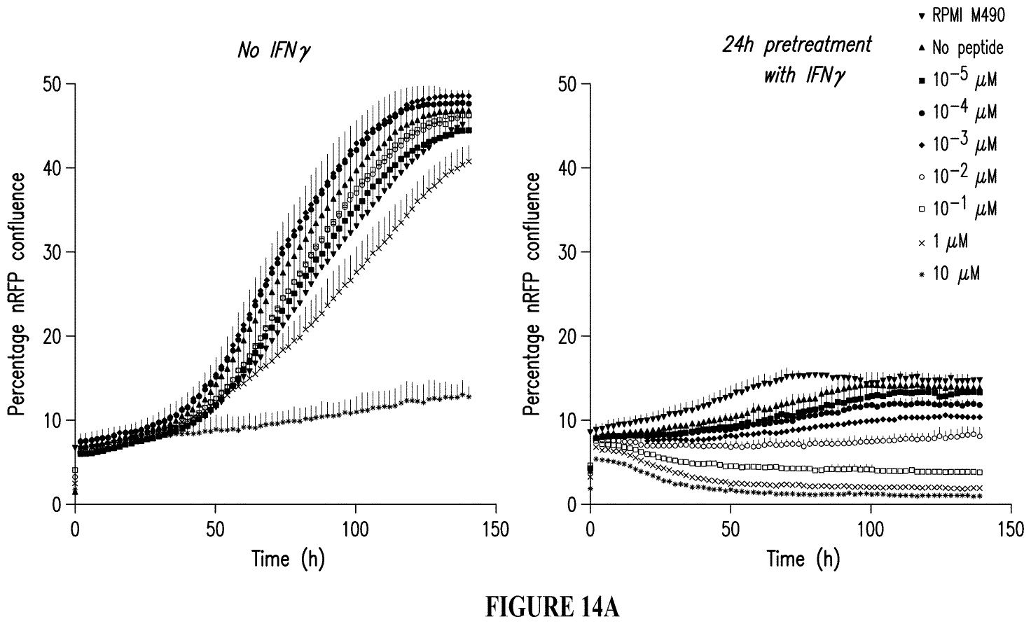

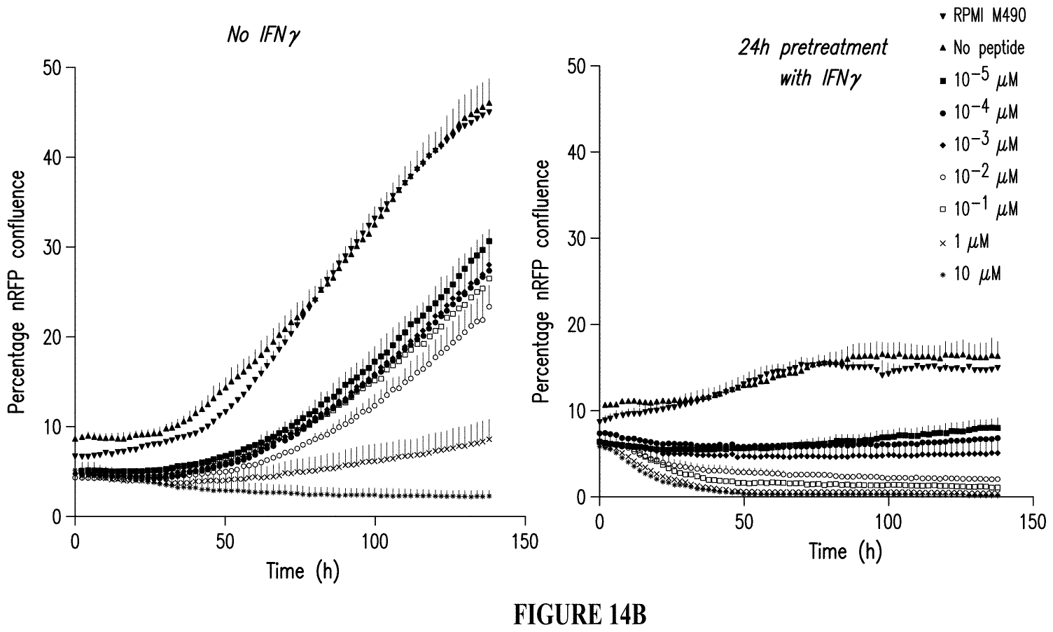

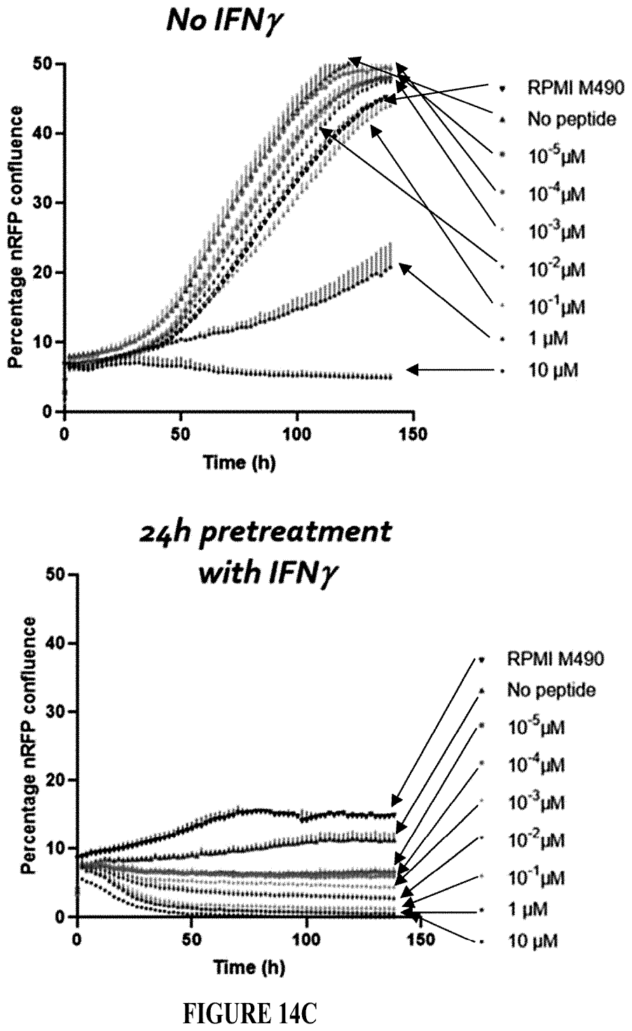

Of note is that HLA-A is the HLA that has the highest expression on the M490 tumor cells as shown in FIG. 9A. Accordingly, NeoTCRs specific to HLA-A are expected to have superior to HLA-B and HLA-C in the M490 tumor cells. Accordingly, the same experiment described above was performed on HLA-B restricted NeoTCR Products. As shown in FIGS. 14A-14C, IFNγ pretreatment had a greater effect on HLA-B restricted NeoTCR Products than it did on HLA-A restricted NeoTCR Products shown in FIGS. 13A and 13B. FIG. 14A=NeoTCR Product expressing NeoTCR 418; FIG. 14B=NeoTCR Product expressing NeoTCR 433; FIG. 14C=NeoTCR Product expressing NeoTCR 423. A description of the NeoTCRs is provided in Table 8 below.

TABLE 8
NeoTCR 418, NeoTCR 433, and NeoTCR 423

| | | | | Dex binding | | %CD4- |
|---|---|---|---|---|---|---|
| NeoTCR | Neoepitope | Gene | HLA | to CD4 cells | %Dex+2A+ | CD8- |
| 418 | LLLGGSLMEY | HP1BP3 | HLA- | No | 21.6 | 1.64 |
| | (SEQ ID NO: 2) | | B15:01 | | | |
| 433 | LLLGGSLMEY | HP1BP3 | HLA- | No | 27.4 | 3.3 |
| | (SEQ ID NO: 2) | | B15:01 | | | |
| 423 | LLLGGSLMEY | HP1BP3 | HLA- | Yes | 41.6 | 8.44 |
| | (SEQ ID NO: 2) | | B15:01 | | | |

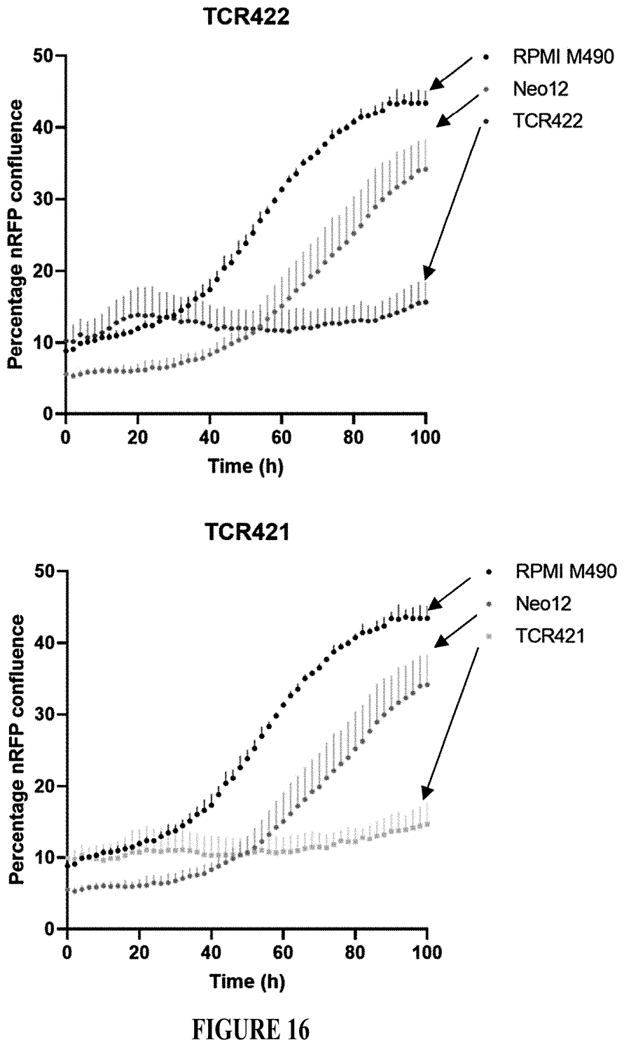

In order to understand why the expression of two or more NeoTCRs in a NeoTCR Product elicited the same effect as peptide pulsing and IFNγ pretreatment, priming experiments were designed (FIG. 15). The priming experiment consisted of culturing the M490 tumor cells for 20 hrs in the presence of a first NeoTCR Product (expressing a single NeoTCR) followed by the removal of that first NeoTCR Product and replacing it with either plain media or media plus a second NeoTCR Product (expressing a single NeoTCR). The question to answer was wither tumor cells that are primed with one NeoTCR will induce an apoptotic signal that will make them more susceptible to subsequent killing by a NeoTCR Product. In the experiments shown in FIG. 16, the tumor cells were primed for 20 hrs with either a NeoTCR Product expressing NeoTCR 422 or NeoTCR 421. After the 20 hrs, the priming NeoTCR Product was removed and replaced with either plain media or a NeoTCR Product expressing the other NeoTCR (e.g., if NeoTCR 422 was used for priming, NeoTCR 421 was used as the replacement product post-priming). As shown, in both instances, the priming resulted in better cell killing and increased control of M490 tumor cell growth. A description of the NeoTCRs is provided in Table 9 below.

TABLE 9
NeoTCR 418, NeoTCR 433, and NeoTCR 423

| | | | | Dex binding | %CD4- |
|---|---|---|---|---|---|
| NeoTCR | Neoepitope | Gene | HLA | to CD4 cells | CD8- |
| 422 | NILPISFHV (SEQ | NAT10 | HLA- | Yes | 46.3 |
| | ID NO: 1) | | A02:01 | | |
| 421 | KAVDISMIL (SEQ | PRPSAP2 | HLA- | Yes | 29.8 |
| | ID NO: 3) | | C03:04 | | |

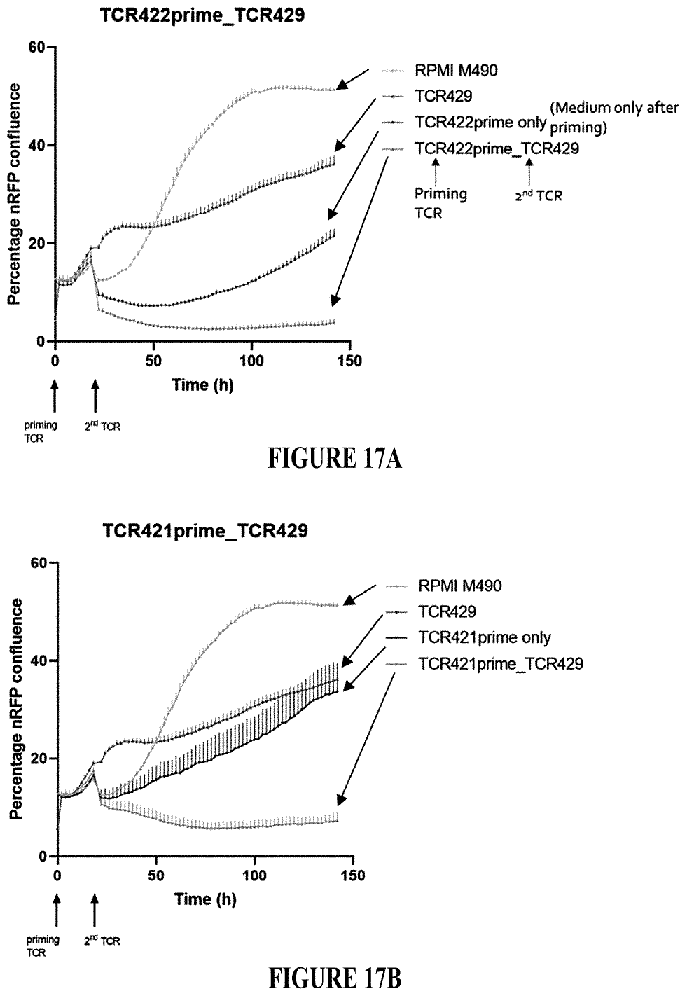

Similar experiments were performed and data is shown in FIGS. 17A (NeoTCR 422 as the primer and NeoTCR 429 in the NeoTCR Product for treatment) and 17B (NeoTCR 421 as the primer and NeoTCR 429 in the NeoTCR Product for treatment). A description of the NeoTCRs used in this experiment is provided in Table 10 below.

TABLE 10
NeoTCR 422, NeoTCR 429, and NeoTCR 421

| | | | | Dex binding | | %CD4- |
|---|---|---|---|---|---|---|
| NeoTCR | Neoepitope | Gene | HLA | to CD4 cells | %Dex+2A+ | CD8- |
| 422 | NAT10 | NILPISFHV (SEQ | HLA- | Yes | 46.3 | 2.04 |
| | | ID NO: 1) | A02:01 | | | |
| 429 | UVSSA | ATTRAVQGWN | HLA- | No | 17 | 1.68 |
| | | (SEQ ID NO: 4) | B57:01 | | | |
| 421 | PRPSAP2 | KAVDISMIL (SEQ | HLA- | Yes | 29.8 | 1.17 |
| | | ID NO: 3) | C03:04 | | | |

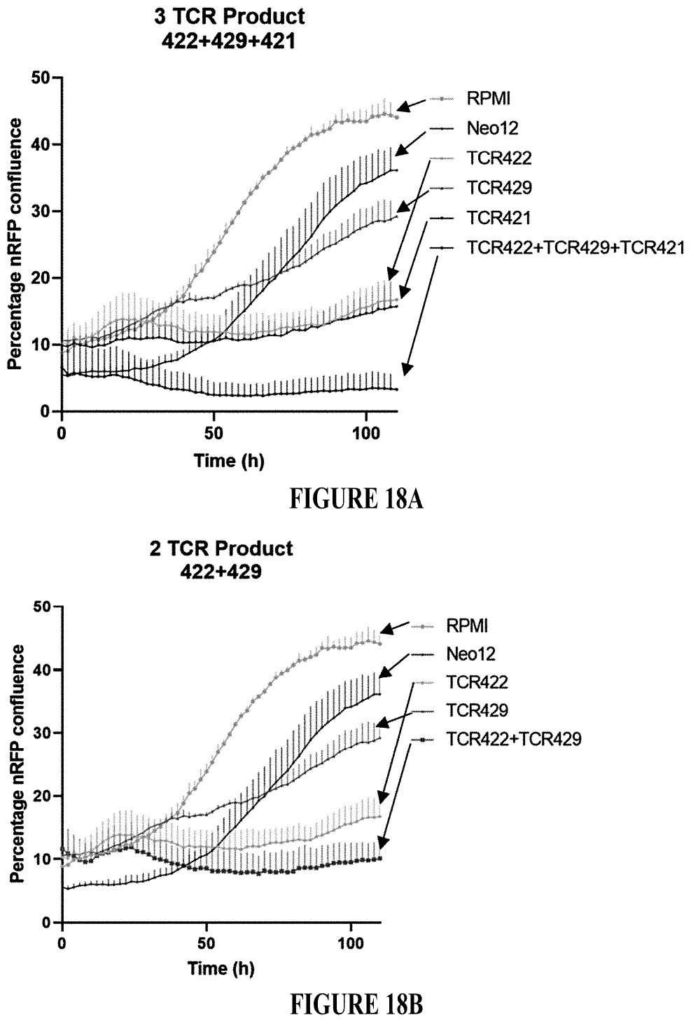

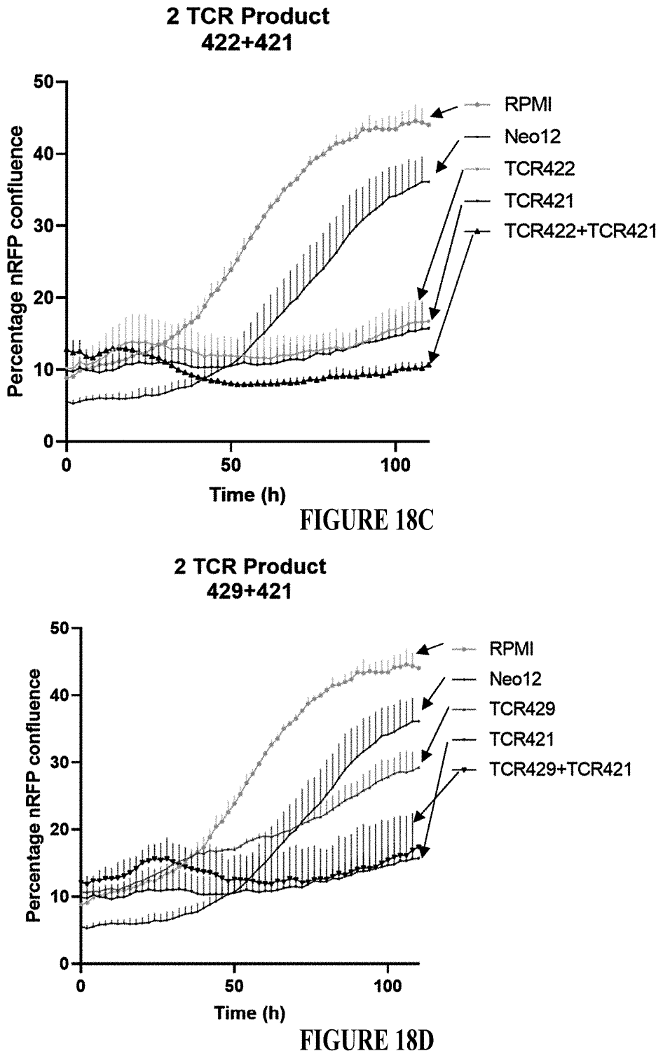

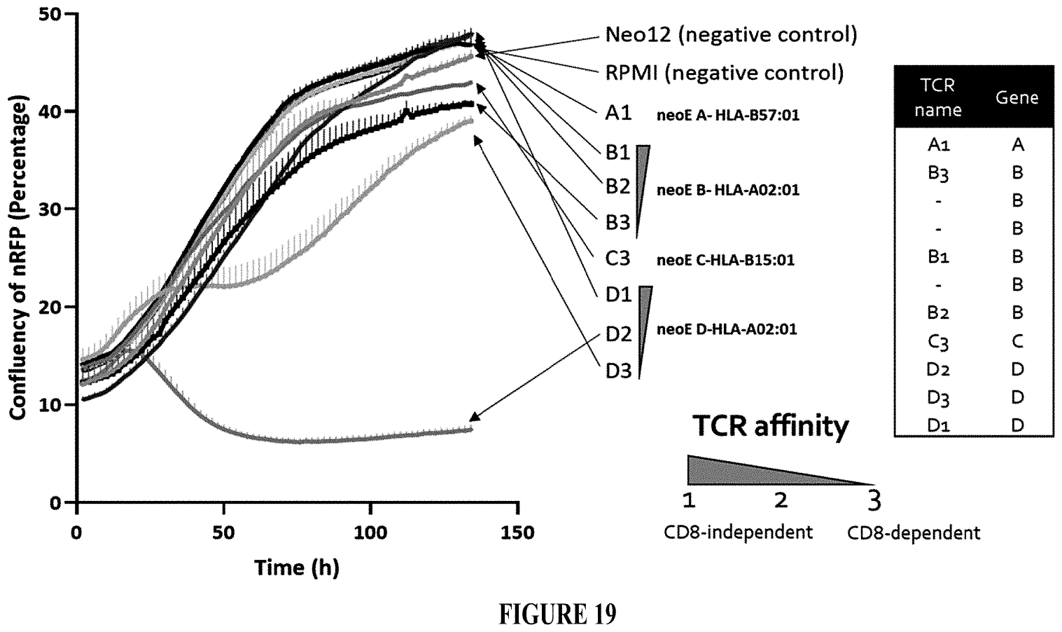

Finally, in order to determine if synergy occurred by the combination of three different NeoTCRs in a NeoTCR Product, the NeoTCRs from the experiments shown in FIGS. 17A and 17B were used to perform a combination study (FIGS. 17A-18D). FIG. 17A shows the single NeoTCR Products compared to the three NeoTCR Product in their abilities to control tumor cell growth. FIGS. 18B-18D show the ability of the combination of two of the three NeoTCRs in NeoTCR Products to control tumor cell growth. As shown, the combination of two or more NeoTCRs in a single NeoTCR Product elicits the same result of the priming experiments proving that the NeoTCRs are able to work synergistically together to increase the efficacy of a NeoTCR Product to control tumor cell growth.

### Example 20. Exemplary Generation of a NeoTCR Product

The NeoTCR Products described herein can be made according to the methods set forth in this example.

Neoepitope-specific TCRs identified by the imPACT Isolation Technology described in PCT/US2020/17887 (which is herein incorporated by reference in its entirety) were used to generate homologous recombination (HR) DNA templates. These HR templates were transfected into primary human T cells in tandem with site-specific nucleases. The single-step non-viral precision genome engineering resulted in the seamless replacement of the endogenous TCR with the patient's neoepitope-specific TCR, expressed by the endogenous promoter. The TCR expressed on the surface is entirely native in sequence.

The precision of neoTCR-T cell genome engineering was evaluated by Targeted Locus Amplification (TLA) for off-target integration hot spots or translocations, and by next generation sequencing based off-target cleavage assays and found to lack evidence of unintended outcomes.

Constructs containing genes of interest were inserted into endogenous loci. This was accomplished with the use of homologous repair templates containing the coding sequence of the gene of interest flanked by left and right HR arms. In addition to the HR arms, the gene of interest was sandwiched between 2A peptides, a protease cleavage site that is upstream of the 2A peptide to remove the 2A peptide from the upstream translated gene of interest, and signal sequences. Once integrated into the genome, the gene of interested expression gene cassette was transcribed as single messenger RNA. During the translation of this gene of interest in messenger RNA, the flanking regions were unlinked from the gene of interest by the self-cleaving 2A peptide and the protease cleavage site was cleaved for the removal of the 2A peptide upstream from the translated gene of interest. In addition to the 2A peptide and protease cleavage site, a gly-ser-gly (GSG) linker was inserted before each 2A peptide to further enhance the separation of the gene of interest from the other elements in the expression cassette.

It was determined that P2A peptides were superior to other 2A peptides for Cell Products because of its efficient cleavage. Accordingly, two (2) P2A peptides and codon divergence were used to express the gene of interest without introducing any exogenous epitopes from remaining amino acids on either end of the gene of interest from the P2A peptide. The benefit of the gene edited cell having no exogenous epitopes (i.e., no flanking P2A peptide amino acids on either side of the gene of interest) is that immunogenicity is drastically decreased and there is less likelihood of a patient infused with a Cell Product containing the gene edited cell to have an immune reaction against the gene edited cell.

As described in PCT/US/2018/058230, NeoTCRs were integrated into the TCRα locus of T cells. Specifically, a homologous repair template containing a NeoTCR coding sequence flanked by left and right HR Arms was used. In addition, the endogenous TCRβ locus was disrupted leading to the expression of only TCR sequences encoded by the NeoTCR construct. The general strategy was applied using circular HR templates as well as with linear templates.

Once integrated into the genome, the NeoTCR expression gene cassette is transcribed as a single messenger RNA from the endogenous TCRα promoter, which still includes a portion of the endogenous TCRα polypeptide from that individual T cell. During ribosomal polypeptide translation of this single NeoTCR messenger RNA, the NeoTCR sequences are unlinked from the endogenous, CRISPR-disrupted TCRα polypeptide by self-cleavage at a P2A peptide. The encoded NeoTCRα and NeoTCRβ polypeptides are also unlinked from each other through cleavage by the endogenous cellular human furin protease and a second self-cleaving P2A sequence motifs included in the NeoTCR expression gene cassette. The NeoTCRα and NeoTCRβ polypeptides are separately targeted by signal leader sequences (derived from the human growth hormone, HGH) to the endoplasmic reticulum for multimer assembly and trafficking of the NeoTCR protein complexes to the T cell surface. The inclusion of the furin protease cleavage site facilitates the removal of the 2A sequence from the upstream TCRβ chain to reduce potential interference with TCRβ function. Inclusion of a gly-ser-gly linker before each 2A further enhances the separation of the three polypeptides.

Additionally, three repeated protein sequences are codon diverged within the HR template to promote genomic stability. The two P2A are codon diverged relative to each other, as well as the two HGH signal sequences relative to each other, within the TCR gene cassette to promote stability of the introduced NeoTCR cassette sequences within the genome of the ex vivo engineered T cells. Similarly, the re-introduced 5′ end of TRAC exon 1 reduces the likelihood of the entire cassette being lost over time through the removal of intervening sequence of two direct repeats.

In-Out PCR was used to confirm the precise target integration of the NeoE TCR cassette. Agarose gels show the results of a PCR using primers specific to the integration cassette and site generate products of the expected size only for cells treated with both nuclease and DNA template (KOKI and KOKIKO), demonstrating site-specific and precise integration.

Furthermore, Targeted Locus Amplification (TLA) was used to confirm the specificity of targeted integration. Crosslinking, ligation, and use of primers specific to the NeoTCR insert were used to obtain sequences around the site(s) of integration. The reads mapped to the genome are binned in 10 kb intervals. Significant read depths were obtained only around the intended site the integration site on chromosome 14, showing no evidence of common off-target insertion sites.

Antibody staining for endogenous TCR and peptide-HLA staining for neoTCR revealed that the engineering results in high frequency knock-in of the NeoTCR, with some TCR− cells and few WT T cells remaining. Knock-in is evidenced by neoTCR expression in the absence of an exogenous promoter. Engineering was carried out multiple times using the same neoTCR with similar results. Therefore, efficient and consistent expression of the NeoTCR and knockout of the endogenous TCR in engineered T cells was achieved.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A composition comprising a first NeoTCR cell population comprising a first patient-derived NeoTCR that binds a first neoantigen, and a second NeoTCR cell population comprising a second patient-derived NeoTCR that binds a second neoantigen; wherein the first and second NeoTCR are different.

2. The composition of claim 1, further comprising a third NeoTCR cell population comprising a third patient-derived NeoTCR that binds a third neoantigen, wherein the third NeoTCR is different from the first and second NeoTCRs.

3. The composition of claim 2, wherein at least two of the first, second, and third neoantigens are expressed by a single gene.

4. The composition of claim 2, wherein the first, second, and third neoantigens are expressed by different genes.

5. The composition of claim 1, wherein at least one of the first, second, and third NeoTCRs binds to a single major histocompatibility complex (MHC).

6. The composition of claim 1, wherein the first, second, and third NeoTCRs bind to different MHCs.

7. The composition of claim 2, wherein at least one of the first, second, and third NeoTCRs binds to a single major histocompatibility complex (MHC).

8. The composition of claim 2, wherein the first, second, and third NeoTCRs bind to different MHCs.

9. A method of treating a cancer in a subject in need thereof, comprising administering the composition comprising a first NeoTCR cell population comprising a first patient-derived NeoTCR that binds a first neoantigen, and a second NeoTCR cell population comprising a second patient-derived NeoTCR that binds a second neoantigen; wherein the first and second NeoTCRs are different.

10. The method of claim 9, further comprising a third NeoTCR cell population comprising a third patient-derived NeoTCR that binds a third neoantigen, wherein the third NeoTCR is different from the first and second NeoTCRs

11. The method of claim 9, wherein the composition comprises an amount of NeoTCR Cells of about 4×10⁸ cells, 1.33×10⁹ cells, or about 4×10⁹ cells.

12. The method of claim 9, wherein the composition comprises an amount of NeoTCR cells greater than about 4×10⁸ cells and less than about 1.33×10⁹ cells, greater than about 1.33×10⁹ cells and less than about 4×10⁹ cells, or greater than about 4×10⁹ cells.

13. The method of claim 9, wherein the composition comprises an amount of NeoTCR cells according to Table 4 or Table 5.

14. The method of claim 9, wherein the composition is administered in a single dose.

15. The method of claim 9, wherein the compositions is administered in multiple doses.

16. The method of claim 9, further comprising administering a combination agent.

17. The method of claim 16, wherein the combination agent is a cytokine, a PD-axis binding agent, a PD-1 binding agent, a PD-L1 binding agent, a PD-L2 binding agent, or a combination thereof.

18. The method of claim 17, wherein the cytokine is an IL-2 agent or an IL-15 agent.

19. The method of claim 17, wherein the PD-axis binding agent comprises nivolumab, pembrolizumab, or atezolizumab.

20. The method of claim 9, wherein the composition is administered following a pretreatment regime of fludarabine and cyclophosphamide.

21. A plurality of cells comprising:
a. a first modified T cell comprising a first exogenous polynucleotide encoding a first patient-derived NeoTCR binding a first neoantigen, wherein the first exogenous polynucleotide is integrated in an endogenous TRAC and/or TRBC locus of the first modified T cell; and
b. a second modified T cell comprising a second exogenous polynucleotide encoding a second patient-derived NeoTCR binding a second neoantigen, wherein the second exogenous polynucleotide is integrated in an endogenous TRAC and/or TRBC locus of the second modified T cell;
wherein the first and second modified T cells are patient-derived.

22. The plurality of cells of claim 21, further comprising a third modified T cell comprising a third exogenous polynucleotide encoding a third patient-derived NeoTCR binding a third neoantigen, wherein the third exogenous polynucleotide is integrated in an endogenous TRAC and/or TRBC locus of the third modified T cell and wherein the third modified T cell is patient-derived.

23. The plurality of cells of claim 21, wherein the T cells are:
a. CD45RA+, CD62L+, CD28+, CD95-, CCR7+, and CD27+;
b. CD45RA+, CD62L+, CD28+, CD95+, CD27+, CCR7+; or
c. CD45RO+, CD62L+, CD28+, CD95+, CCR7+, CD27+, CD127+.

24. A method of treating a cancer in a subject in need thereof, the method comprising administering a plurality of cells, comprising:
a. a first modified T cell comprising a first exogenous polynucleotide encoding a first patient-derived NeoTCR binding a first neoantigen, wherein the first exogenous polynucleotide is integrated in an endogenous TRAC and/or TRBC locus of the first modified T cell; and
b. a second modified T cell comprising a second exogenous polynucleotide encoding a second patient-derived NeoTCR binding a second neoantigen, wherein the second exogenous polynucleotide is integrated in an endogenous TRAC and/or TRBC locus of the second modified T cell;
wherein the first and second modified T cells are patient derived, thereby treating the cancer in the subject.

25. The method of claim 24, further comprising a third modified T cell comprising a third exogenous polynucleotide encoding a third patient-derived NeoTCR binding a third neoantigen, wherein the third exogenous polynucleotide is integrated in an endogenous TRAC and/or TRBC locus and wherein the third modified T cell is patient-derived.

26. The method of claim 24, wherein the T cells are:
a. CD45RA+, CD62L+, CD28+, CD95−, CCR7+, and CD27+;
b. CD45RA+, CD62L+, CD28+, CD95+, CD27+, CCR7+; or
c. CD45RO+, CD62L+, CD28+, CD95+, CCR7+, CD27+, CD127+.

27. The method of claim 24, further comprising administering a combination agent.

28. The method of claim 27, wherein the combination agent is a cytokine, a PD-axis binding agent, a PD-1 binding agent, a PD-L1 binding agent, a PD-L2 binding agent, or a combination thereof.

29. The method of claim 28, wherein the cytokine is an IL-2 agent or an IL-15 agent.

30. The method of claim 28, wherein the PD-axis binding agent comprises nivolumab, pembrolizumab, or atezolizumab.


SEQUENCE LISTING (4 sequences)

SEQ ID NO: 1 - protein, 9 aa, Artificial Sequence
  Synthetic
NILPISFHV

SEQ ID NO: 2 - protein, 10 aa, Artificial Sequence
  Synthetic
LLLGGSLMEY

SEQ ID NO: 3 - protein, 9 aa, Artificial Sequence
  Synthetic
KAVDISMIL

SEQ ID NO: 4 - protein, 10 aa, Artificial Sequence
  Synthetic
ATTRAVQGWN